(19) United States
(12) Patent Application Publication
(10) Pub. No.: US 2026/0158051 A1
(43) Pub. Date: Jun. 11, 2026

(54) Compositions and Methods for Treating and Preventing Prekallikrein-Associated Conditions

(71) Applicant: Ionis Pharmaceuticals, Inc., Carlsbad, CA (US)

(72) Inventors: Eugene SCHNEIDER, San Marcos, CA (US); Nicholas J. VINEY, Carlsbad, CA (US); Veronica J. ALEXANDER, San Diego, CA (US); Laura BORDONE, Cardiff, CA (US); Kenneth NEWMAN, Carlsbad, CA (US)

(73) Assignee: Ionis Pharmaceuticals, Inc., Carlsbad, CA (US)

(21) Appl. No.: 19/271,374

(22) Filed: Jul. 16, 2025

Related U.S. Application Data
(63) Continuation of application No. 17/908,773.
(60) Provisional application No. 62/989,427, filed on Mar. 13, 2020.

(51) Int. Cl. A61K 31/7088; C12N 15/113

(52) U.S. Cl. CPC A61K 31/7088; C12N 15/1137; C12N 2310/315; C12N 2310/322; C12N 2310/3341; C12N 2310/341

(57) ABSTRACT

Provided herein are methods of administering ISIS 721744 for ameliorating edema, and methods of reducing prekallikrein (PKK) RNA, protein or activity in a human subject in need thereof. In certain instances, methods are useful for ameliorating at least one symptom of hereditary angioedema. Such symptoms of hereditary angioedema include, but are not limited to, nausea, vomiting, itching, headache, fatigue, abdominal pain, shortness of breath, rhinitis, anaphylaxis, bronchoconstriction, and swelling. In certain instances, methods are useful for ameliorating at least one symptom of macular edema. Such symptoms of macular edema include, but are not limited to, impaired vision and vision loss

### CROSS-REFERENCE TO RELATED APPLICATIONS

This application is a continuation of U.S. application Ser. No. 17/908,773 filed Sep. 1, 2022, which is a U.S. National Stage of International Application PCT/US2021/022069 filed Mar. 12, 2021, which claims priority to U.S. Provisional Application No. 62/989,427 filed Mar. 13, 2020, the entireties of each of which are incorporated by reference herein.

### SEQUENCE LISTING

The present application is being filed along with a Sequence Listing in electronic format. The Sequence Listing is provided as a file entitled BIOL0377SEQ.xml, created Sep. 22, 2025, which is 53,478 bytes in size. The information in the electronic format of the sequence listing is incorporated herein by reference in its entirety.

### FIELD

Provided herein are methods of administering ISIS 721744 for ameliorating edema, reducing prekallikrein (PKK) RNA, reducing PKK protein, and/or reducing PKK activity in a human subject in need thereof. In certain instances, methods are useful for ameliorating at least one symptom of hereditary angioedema. Such symptoms of hereditary angioedema include, but are not limited to, swelling, nausea, vomiting, itching, headache, fatigue, abdominal pain, shortness of breath, rhinitis, anaphylaxis, and bronchoconstriction. In certain instances, methods are useful for ameliorating at least one symptom of macular edema. Such symptoms of macular edema include, but are not limited to, blurry vision, wavy vision, distorted vision, and vision loss.

### BACKGROUND

Prekallikrein (PKK) is a glycoprotein encoded by the KLKB1 gene and is cleaved from its zymogen form to an enzyme form, referred to as plasma kallikrein (PK), upon activation of Factor XII. PK is a member of the kinin-kallikrein pathway, which plays a role in inflammation, blood pressure control, coagulation, and pain. PK generates plasmin from plasminogen and liberates kinins (e.g., bradykinin) from kininogens. In addition, PK can in turn reciprocally activate FXII and thereby enhance bradykinin release.

Edema is a medical term for the swelling of one or more body parts, including extremities, torso, and airway (e.g., sinuses, mouth, throat, lungs). Edema occurs when an injury, inflammation, or other condition (e.g., low albumin, allergies, congestive heart failure, diabetes, genetic mutations) causes small blood vessels to leak fluid into nearby tissues. There are many types of edema, including, but not limited to, hereditary angioedema (HAE), pulmonary edema, cerebral edema, and macular edema.

HAE is a rare autosomal dominant disease characterized by recurrent and unpredictable attacks of swelling, particularly of the skin, gastric system, oropharyngeal tissue, and laryngeal mucosa, which can be life threatening. The typical swellings in HAE are caused by locally increased vascular permeability and fluid efflux in response to excessive bradykinin formation which results from inadequate control of the contact system components. In most cases, HAE is caused by genetic mutations in the gene, SERPING1, which encodes C1 esterase inhibitor (C1-INH) that regulates the kinin-kallikrein pathway. These genetic mutations lead to a C1-INH deficiency (Type I HAE) or dysfunction (Type II HAE). The SERPING1 gene encodes C1-esterase inhibitor (C1-INH).

In a rare, third type of HAE, Type III HAE, also known as normal level (nl)-C1-INH-HAE, patients have normal levels of functional C1-INH and often have a distinct clinical presentation, including a higher frequency of facial, pharyngeal, and tongue swellings. There are at least four subtypes of Type III HAE: a subtype associated with a genetic mutation (e.g., Thr328Lys, Thr328Arg, Thr309Lys, and Thr309Arg) in the FXII gene (F12), also known as “HAE-FXII;” a subtype associated with a genetic mutation (e.g., Lys330Glu) in the plasminogen gene (PLG), also known as “HAE-PLG;” a subtype associated with a genetic mutation (e.g., Ala119Ser) in the angiopoietin-1 gene (ANGPT1), also known as “HAE-ANGPT1;” and a subtype not associated with a mutation in F12, PLG, or ANGPT1, also known as “HAE-UI.”

Macular edema is a condition wherein fluid collects in the retina, including the macula, causing blurry, distorted, or wavy vision. In some cases, macular edema can lead to partial or total vision loss. Macular edema may be caused by or associated with eye surgery, macular degeneration, retinal blood vessel blockage, an infection of the eye, and inflammation of the eye. Macular edema often occurs as a result of diabetic neuropathy, which is a complication of diabetes that is characterized by damaged and leaky blood vessels in and/or near the retina. Macular edema associated with diabetic neuropathy is referred to as diabetic macular edema (DME). DME is the most common cause of vision loss or vision impairment in patients with diabetic retinopathy.

### BRIEF DESCRIPTION OF THE DRAWINGS

FIG. 1A shows mean concentration of PKK protein in plasma as percent change from baseline at multiple time points during monthly dosing of ISIS 721744 in healthy volunteers.

FIG. 1B shows mean plasma proenzyme activation (a measure of the plasma's ability to generate bradykinin) as percent change from baseline at multiple time points during monthly dosing of ISIS 721744 in healthy volunteers.

### SUMMARY

Provided herein are methods for ameliorating edema, and methods of reducing PKK RNA, PKK protein, and/or PKK activity in a human subject in need thereof. In certain embodiments, edema comprises hereditary angioedema. In certain embodiments, edema comprises macular edema. In certain embodiments, methods comprise administering a therapeutically effective amount of an oligomeric compound. In certain embodiments, the oligomeric compound is ISIS 721744. In certain embodiments, the therapeutically effective amount is within the range of about 40 mg to about 120 mg. In certain embodiments, the therapeutically effective amount is about 80 mg. In certain embodiments, the therapeutically effective amount is about 100 mg. In certain embodiments, the therapeutically effective amount is administered once about every 2 weeks. In certain embodiments, the therapeutically effective amount is administered once about every 4 weeks. In certain embodiments, the therapeutically effective amount is administered once about every 8 weeks. In certain embodiments, methods comprise administering a loading dose of about 80 mg of ISIS 721744 once about every 4 weeks, and subsequently administering a maintenance dose of 100 mg of ISIS 721744 once about every 4 weeks. In certain embodiments, methods comprise administering a loading dose of about 80 mg of ISIS 721744 once about every 4 weeks, and subsequently administering a maintenance dose of 80 mg of ISIS 721744 once about every 8 weeks. In certain embodiments, methods comprise administering a loading dose of about 80 mg of ISIS 721744 once about every 2 weeks, and subsequently administering a maintenance dose of 80 mg of ISIS 721744 once about every 4 weeks.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxyribonucleoside” means a nucleoside comprising a 2′-H(H) deoxyribosyl sugar moiety. In certain embodiments, a 2′-deoxyribonucleoside is a 2′-β-D deoxyribonucleoside and comprises a 2′-β-D-deoxyribosyl sugar moiety, which has the β-D configuration as found in naturally occurring deoxyribonucleic acids (DNA). In certain embodiments, a 2′-deoxyribonucleoside may comprise a modified nucleobase or may comprise an RNA nucleobase (uracil).

As used herein, “2′-MOE” means a 2′-OCH₂CH₂OCH₃ group in place of the 2′-OH group of a ribosyl sugar moiety. A “2′-MOE sugar moiety” is a sugar moiety with a 2′-OCH₂CH₂OCH₃ group in place of the 2′-OH group of a ribosyl sugar moiety. Unless otherwise indicated, a 2′-MOE sugar moiety is in the β-D configuration. “MOE” means O-methoxyethyl.

As used herein, “2′-MOE nucleoside” means a nucleoside comprising a 2′-MOE sugar moiety.

As used herein, “5-methyl cytosine” means a cytosine modified with a methyl group attached to the 5 position. A 5-methyl cytosine is a modified nucleobase.

As used herein, “about” means plus or minus 7% of the provided value.

As used herein, “administering” means providing a pharmaceutical agent to a human subject.

As used herein, “ameliorate” in reference to a treatment means improvement in at least one symptom relative to the same symptom in the absence of the treatment. In certain embodiments, amelioration is the reduction in the severity or frequency of a symptom, or the delayed onset or slowing of progression in the severity or frequency of a symptom.

As used herein, “angioedema attack” means a swelling of a body part of a subject having HAE. In certain embodiments, the subject does not have an injury or infection at swelling onset. As used herein, a “breakthrough attack” means an angioedema attack that occurs when the subject is being treated prophylactically for HAE.

As used herein, “anti-edema agent” means a pharmaceutical agent that reduces, ameliorates or prevents edema of a body part of a subject in need thereof.

As used herein, “anti-inflammatory agent” means a pharmaceutical agent that ameliorates or prevents an inflammatory condition in a subject in need thereof.

As used herein, “anti-thrombotic agent” means a pharmaceutical agent that ameliorates or prevents a thrombotic condition in a subject in need thereof.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or group of atoms comprising at least one bond that connects a conjugate moiety to an oligonucleotide. In certain embodiments, a conjugate linker comprises a cleavable moiety.

As used herein, “conjugate moiety” means a group of atoms that is attached to an oligonucleotide via a conjugate linker. In certain embodiments, a conjugate moiety comprises a cell-targeting moiety.

As used herein, “dose” means a quantity of a pharmaceutical agent administered.

As used herein, “edema” means the swelling of one or more body parts. In certain embodiments, the body part is an extremity, e.g., hand, foot, arm, leg, face. In certain embodiments, the body part is not an extremity, e.g., abdomen, tongue, eye. In certain embodiments, the body part facilitates breathing, e.g., lung, trachea, sinus.

As used herein, “PKK RNA” is the RNA expression product of the human gene, KLKB1.

As used herein, “PKK protein” is the protein expression product of PKK RNA.

As used herein, “PKK activity” is the activity of PKK protein in an activated partial thromboplastin time (aPTT) test, a test that characterizes coagulation of a subject's blood. PKK activity in a test plasma sample from the subject is assayed in an aPTT test by adding the test plasma sample to a control plasma sample that is immunodepleted of PKK and comparing clotting (time) of the resulting combined sample to one or more reference plasma samples with a reference amount of PKK to determine relative PKK activity.

As used herein, “inflammatory condition” means a condition in which a body part of a subject becomes reddened, swollen, painful, dysfunctional, or a combination thereof. In certain embodiments, the inflammatory condition comprises any of redness (rubor), increased temperature (calor), swelling (tumor), pain (dolor), or loss of function of the body part. In certain embodiments, the subject has a fever (body temperature above 37° C.). In certain embodiments, the inflammatory condition is diabetes, arthritis, asthma, hepatitis, ulcerative colitis, and Crohn's disease, injury, or infection.

As used herein, the term “internucleoside linkage” means the covalent linkage between contiguous nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “KLKB1 gene” refers to a genomic sequence encoding a PKK RNA. In general, a human has two KLKB1 genes which may have the same or different nucleobase sequences.

As used herein, “loading dose” means a therapeutically effective amount of a pharmaceutical agent administered during an initial dosing phase during which steady state concentration of the pharmaceutical agent is achieved. “Initial loading dose” means the first loading dose administered. “Last loading dose” means the loading dose administered most recently prior to administering a first maintenance dose.

As used herein, “maintenance dose” means a therapeutically effective amount of a pharmaceutical agent administered during a dosing phase after steady state concentration of the pharmaceutical agent has been achieved.

As used herein, “modified oligonucleotide” means an oligonucleotide, wherein at least one nucleoside or internucleoside linkage is modified.

As used herein, “nucleobase” means an unmodified nucleobase or modified nucleobase. An “unmodified nucleobase” is adenine (A), thymine (T), cytosine (C), uracil (U), or guanine (G). A “modified nucleobase” is group of atoms other than unmodified A, T, C, U, or G capable of pairing with at least one unmodified nucleobase. A “5-methyl cytosine” is a modified nucleobase. As used herein, “nucleobase sequence” means the order of contiguous nucleobases in a target nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. “Linked nucleosides” are nucleosides that are connected in a contiguous sequence (i.e., no additional nucleosides are presented between those that are linked). As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides.

As used herein, “oligomeric compound” means a compound comprising an oligonucleotide and a conjugate group.

As used herein, “pharmaceutically acceptable carrier or diluent” means any substance suitable for use in administering to a human subject. Certain such carriers enable pharmaceutical compositions to be formulated as, for example, tablets, pills, dragees, capsules, liquids, gels, syrups, slurries, suspension, and lozenges for the oral ingestion by a human subject. In certain embodiments, a pharmaceutically acceptable carrier or diluent is sterile water, sterile saline, or sterile buffer solution.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “potassium salt” means a salt of an oligomeric compound, wherein the cation of the salt is potassium.

As used herein, “refractory” means resistant to a therapy or pharmaceutical agent used to treat a condition. In certain embodiments, a subject who is refractory to a therapy or pharmaceutical agent has one or more symptoms of the condition after receiving the therapy or pharmaceutical agent. In certain embodiments, the severity of one or more symptoms of the condition is not reduced by the therapy or pharmaceutical agent. In certain embodiments, the occurrence of one or more symptoms of the condition is not reduced by the therapy or pharmaceutical agent.

As used herein, “RNA” means an RNA transcript and includes pre-mRNA and mature mRNA unless otherwise specified.

As used herein, “sodium salt” means a salt of an oligomeric compound, wherein the cation of the salt is sodium.

As used herein, “subject” means a human or non-human animal. In certain embodiments, the subject is a human subject. A “subject in need thereof,” is a subject who would benefit from administration of an oligomeric compound disclosed herein. In certain embodiments, the subject in need thereof has edema.

As used herein, “sugar moiety” means an unmodified sugar moiety or a modified sugar moiety. “Unmodified sugar moiety” means a 2′-OH(H) β-D ribosyl moiety, as found in RNA (an “unmodified RNA sugar moiety”), or a 2′-H(H) β-D deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate.

As used herein, “symptom” means any physical feature or test result that indicates the existence or extent of a disease or disorder. In certain embodiments, a symptom is apparent to a subject or to a medical professional examining or testing the subject.

As used herein, “therapeutically effective amount” means an amount of a pharmaceutical agent that provides a therapeutic benefit to a human subject. For example, a therapeutically effective amount improves a symptom of a disease.

As used herein, “thromboembolic condition” means any disease or condition involving an embolism caused by a thrombus. Examples of such diseases and conditions include the categories of thrombosis, embolism, and thromboembolism. Thromboembolic conditions may also be referred to as thromboembolic events or thrombotic events. Subjects at risk for a thromboembolic condition include, but are not limited to, subjects having hypertension, hypercholesterolemia, atrial fibrillation, a heart valve disorder, valvular heart disease, a mechanical heart valve, surgery, cancer, pregnancy, old age, use of oral contraceptives, immobility, sepsis, atrial fibrillation, atherosclerosis, coronary artery disease (CAD), antiphospholipid syndrome, a prothrombotic clotting disorder (e.g., Factor V Leiden thrombophilia), or a combination thereof.

As used herein, “week” means 7 days.

### Certain Embodiments

The present disclosure provides the following non-limiting numbered embodiments:

Embodiment 1. A method of ameliorating edema in a human subject in need thereof, the method comprising administering to the human subject a therapeutically effective amount of an oligomeric compound according to the following chemical structure:

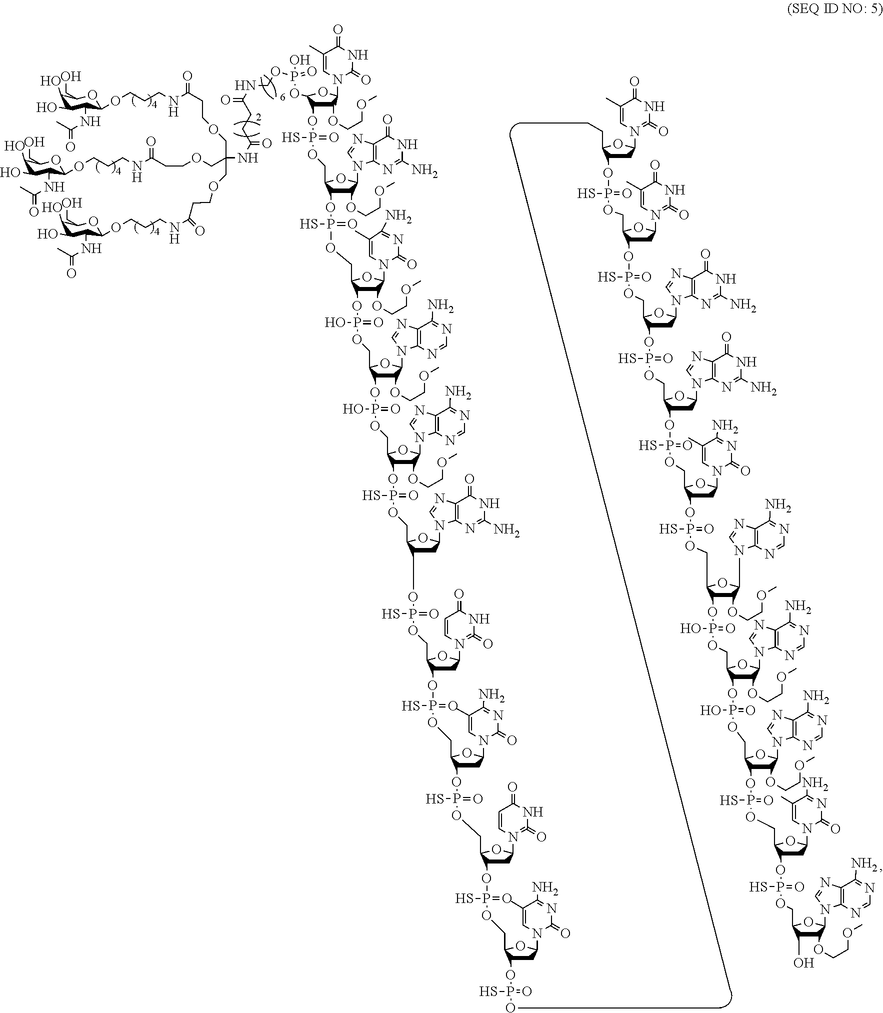

or a salt thereof.

Embodiment 2. The method of embodiment 1, wherein the salt is the sodium salt or the potassium salt.

Embodiment 3. A method of ameliorating edema in a human subject in need thereof, the method comprising administering to the human subject a therapeutically effective amount of an oligomeric compound according to the following chemical structure:

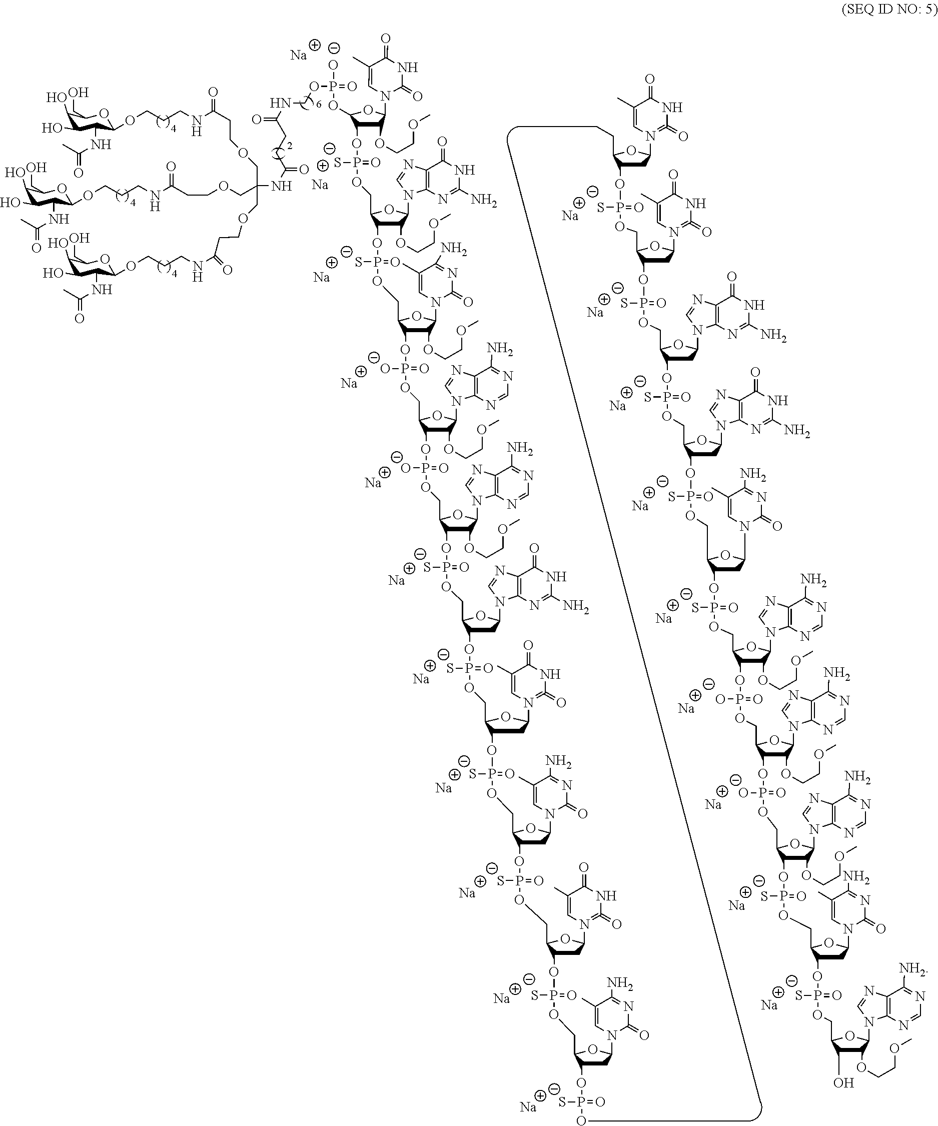

Embodiment 4. A method of ameliorating edema in a human subject in need thereof, the method comprising administering to the human subject a therapeutically effective amount of an oligomeric compound, wherein the oligomeric compound has the following chemical notation (5′ to 3′): (THA-GalNAc3)o Tes Ges mCeo Aeo Aes Gds Tds mCds Tds mCds Tds Tds Gds Gds mCds Aeo Aeo Aes mCes Ae (SEQ ID NO: 5); wherein (THA-GalNAc3)o is represented by the following structure:

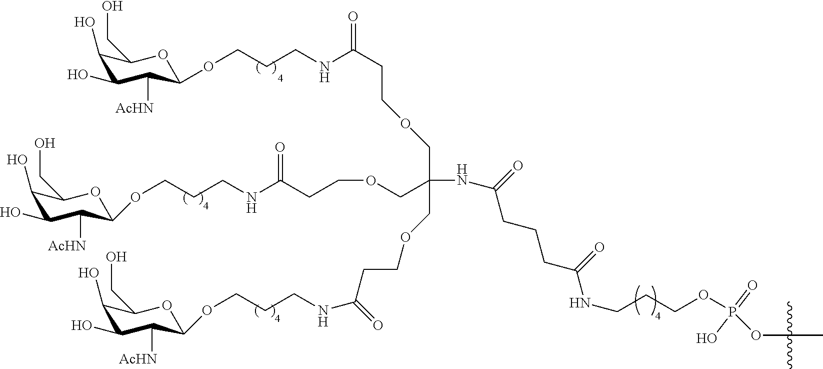

and wherein,

$$A = an adenine nucleobase ,$$

$$mC = a 5 - methyl cytosine nucleobase ,$$

$$G = a guanine nucleobase ,$$

$$T = a thymine nucleobase ,$$

$$e = a 2 ’ - MOE sugar moiety ,$$

$$d = a 2 ’ - a 2 ’ - β - D - deoxyribosyl sugar moiety ,$$

$$s = a phosphorothioate internucleoside linkage , and$$

$$o = a phosphodiester internucleoside linkage .$$

Embodiment 5. The method of any one of embodiments 1-4, wherein edema is hereditary angioedema, and at least one symptom of hereditary angioedema is ameliorated.

Embodiment 6. The method of embodiment 5, wherein at least one symptom is selected from nausea, vomiting, itching, headache, fatigue, abdominal pain, shortness of breath, rhinitis, anaphylaxis, bronchoconstriction, and swelling, and a combination thereof.

Embodiment 7. The method of any one of embodiments 1-4, wherein edema is macular edema, and at least one symptom of macular edema is ameliorated.

Embodiment 8. The method of embodiment 7, wherein at least one symptom is selected from blurry vision, wavy vision, distorted vision, and loss of vision, and a combination thereof.

Embodiment 9. A method of reducing prekallikrein (PKK) RNA in a human subject in need thereof, the method comprising administering to the human subject a therapeutically effective amount of an oligomeric compound according to the following chemical structure:

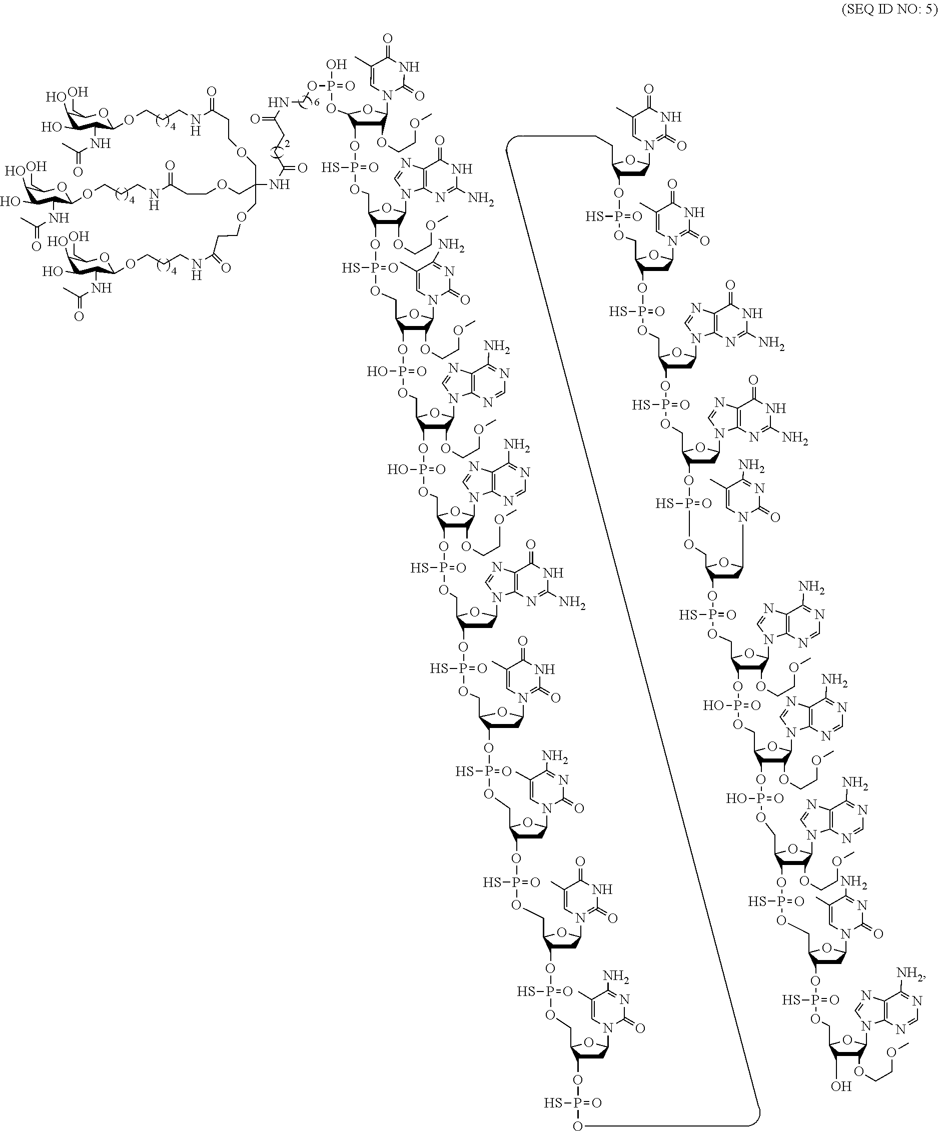

or a salt thereof.

Embodiment 10. The method of embodiment 9, wherein the salt is the sodium salt or the potassium salt.

Embodiment 11. A method of reducing PKK RNA in a human subject in need thereof, the method comprising administering to the human subject a therapeutically effective amount of an oligomeric compound according to the following chemical structure:

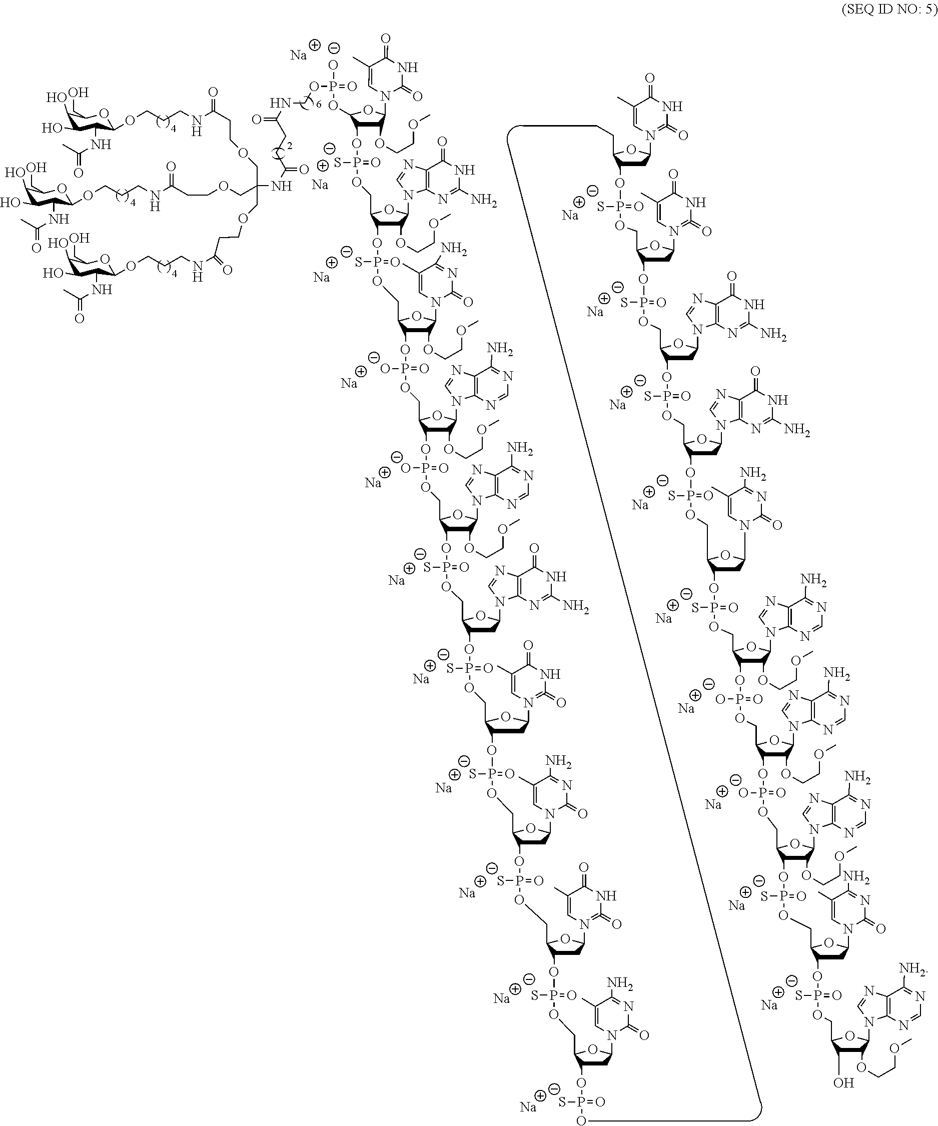

Embodiment 12. A method of reducing PKK RNA in a human subject in need thereof, the method comprising administering to the human subject a therapeutically effective amount of an oligomeric compound, wherein the oligomeric compound has the following chemical notation (5′ to 3′): (THA-GalNAc3)o Tes Ges mCeo Aeo Aes Gds Tds mCds Tds mCds Tds Tds Gds Gds mCds Aeo Aeo Aes mCes Ae (SEQ ID NO: 5); wherein (THA-GalNAc3)o is represented by the following structure:

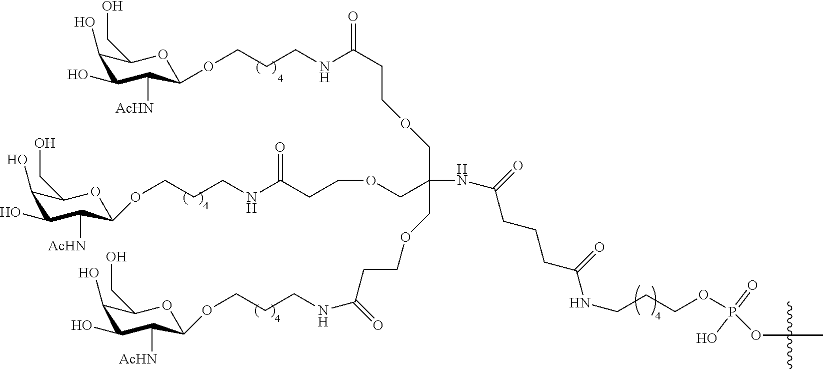

and wherein,

$$A = an adenine nucleobase ,$$

$$mC = a 5 - methyl cytosine nucleobase ,$$

$$G = a guanine nucleobase ,$$

$$T = a thymine nucleobase ,$$

$$e = a 2 ’ - MOE sugar moiety ,$$

$$d = a 2 ’ - a 2 ’ - β - D - deoxyribosyl sugar moiety ,$$

$$s = a phosphorothioate internucleoside linkage , and$$

$$o = a phosphodiester internucleoside linkage .$$

Embodiment 13. A method of reducing PKK protein in a human subject in need thereof, the method comprising administering to the human subject a therapeutically effective amount of an oligomeric compound according to the following chemical structure:

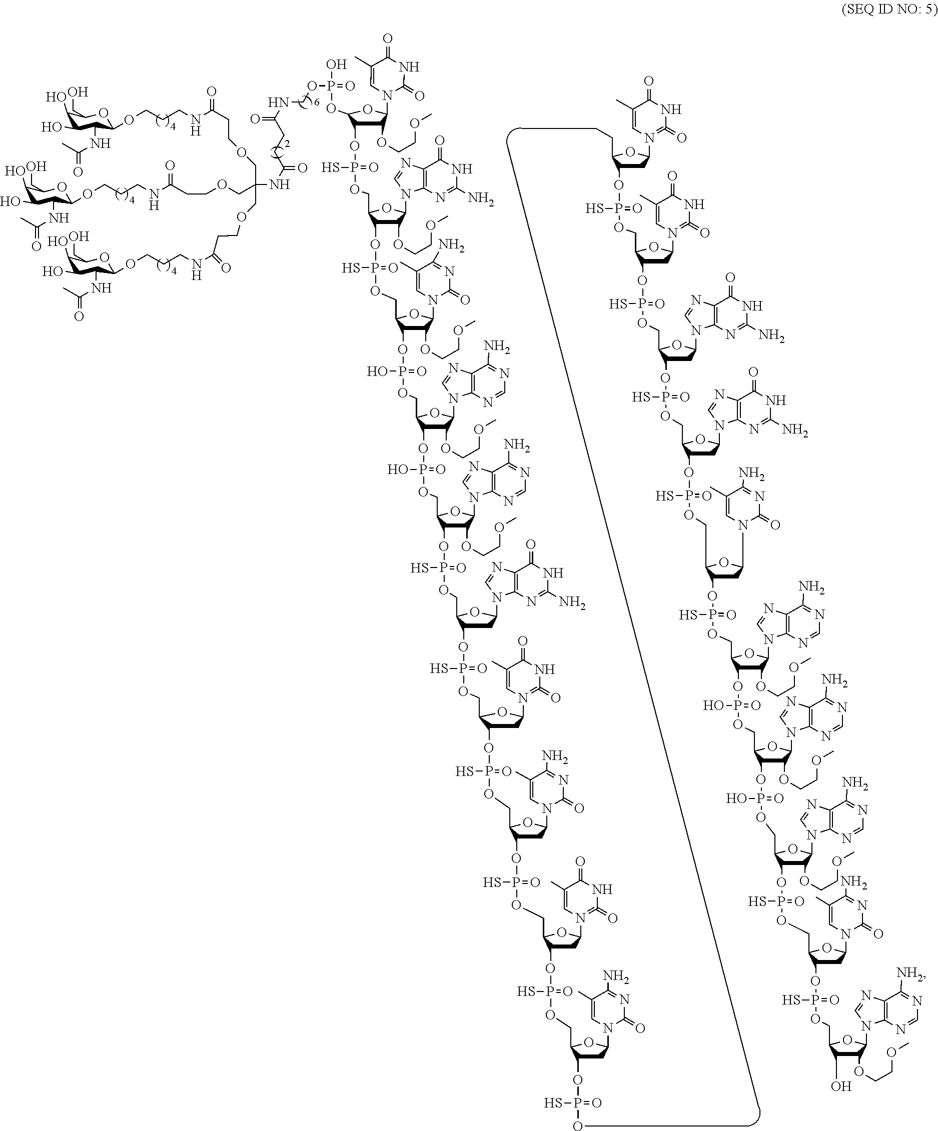

or a salt thereof.

Embodiment 14. The method of embodiment 13, wherein the salt is the sodium salt or the potassium salt.

Embodiment 15. A method of reducing PKK protein in a human subject in need thereof, the method comprising administering to the human subject a therapeutically effective amount of an oligomeric compound according to the following chemical structure:

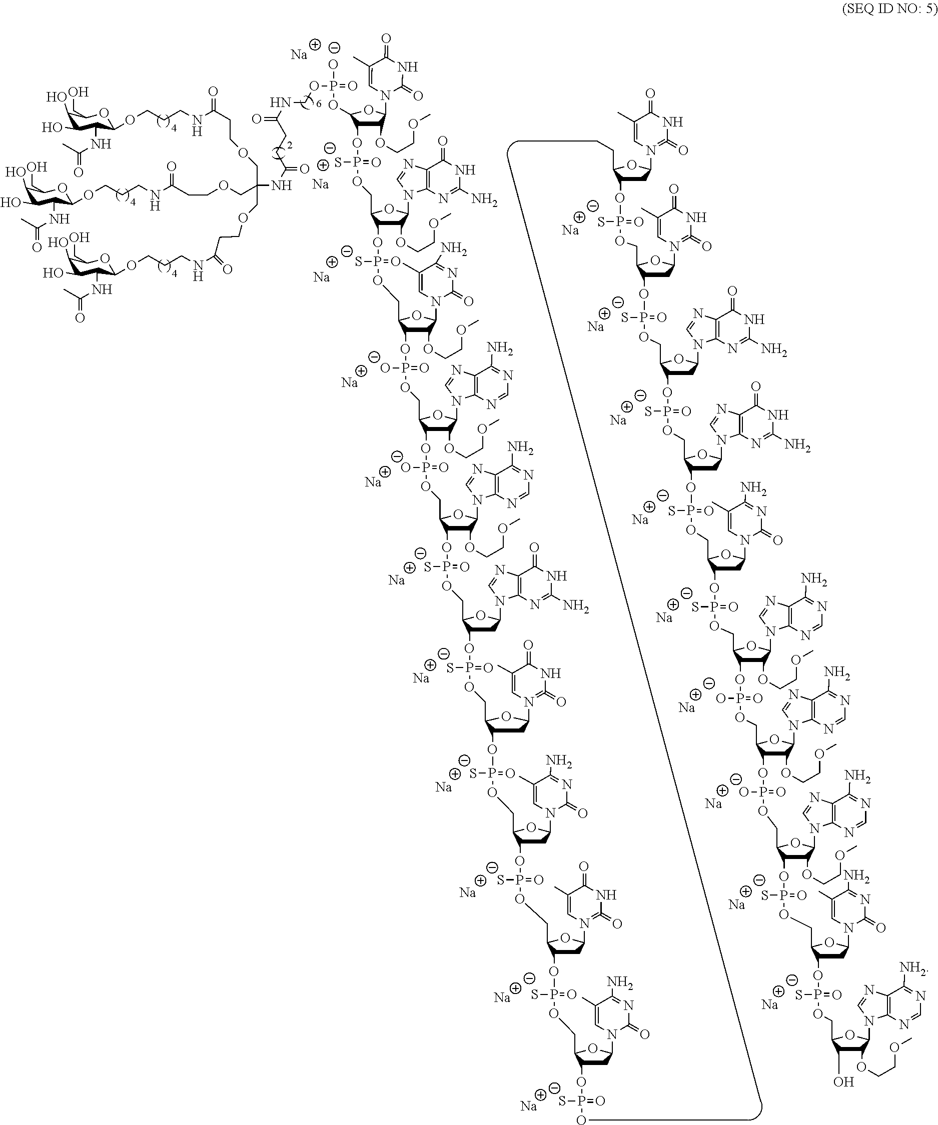

Embodiment 16. A method of reducing PKK protein in a human subject in need thereof, the method comprising administering to the human subject a therapeutically effective amount of an oligomeric compound, wherein the oligomeric compound has the following chemical notation (5′ to 3′): (THA-GalNAc3)o Tes Ges mCeo Aeo Aes Gds Tds mCds Tds mCds Tds Tds Gds Gds mCds Aeo Aeo Aes mCes Ae (SEQ ID NO: 5); wherein (THA-GalNAc3)o is represented by the following structure:

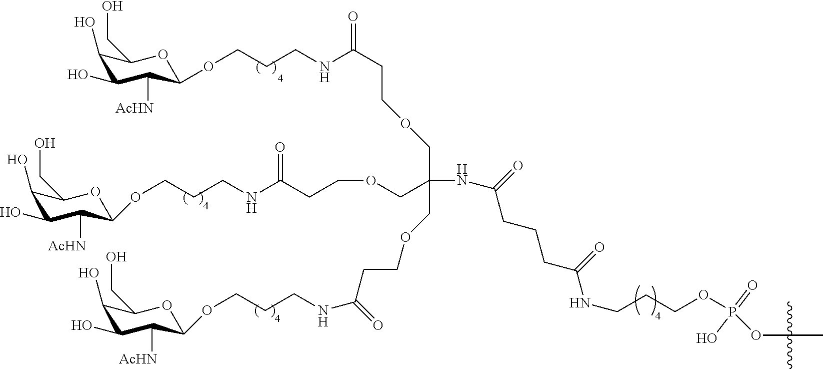

and wherein,

$$A = an adenine nucleobase ,$$

$$mC = a 5 - methyl cytosine nucleobase ,$$

$$G = a guanine nucleobase ,$$

$$T = a thymine nucleobase ,$$

$$e = a 2 ’ - MOE sugar moiety ,$$

$$d = a 2 ’ - a 2 ’ - β - D - deoxyribosyl sugar moiety ,$$

$$s = a phosphorothioate internucleoside linkage , and$$

$$o = a phosphodiester internucleoside linkage .$$

Embodiment 17. A method of reducing PKK activity in a human subject in need thereof, the method comprising administering to the human subject a therapeutically effective amount of an oligomeric compound according to the following chemical structure:

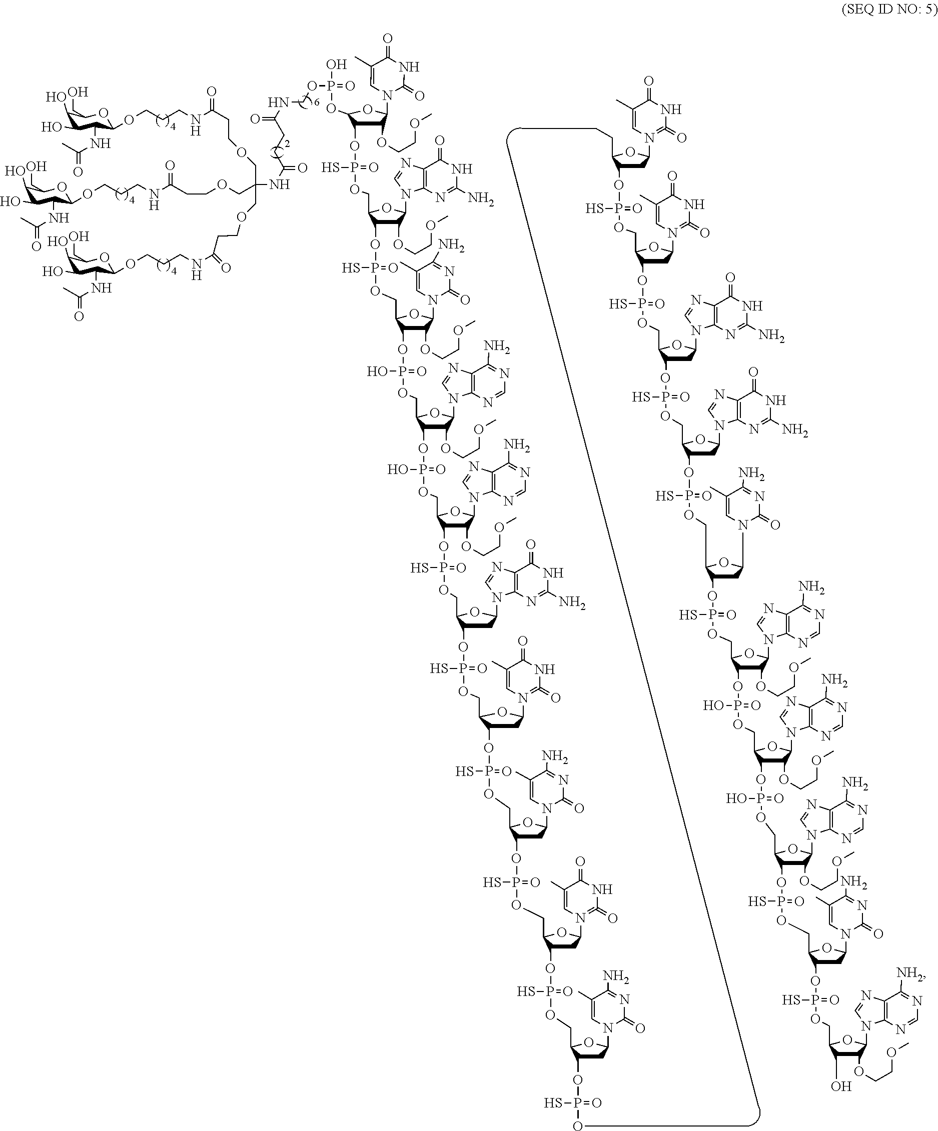

or a salt thereof.

Embodiment 18. The method of embodiment 17, wherein the salt is the sodium salt or the potassium salt.

Embodiment 19. A method of reducing PKK activity in a human subject in need thereof, the method comprising administering to the human subject a therapeutically effective amount of an oligomeric compound according to the following chemical structure:

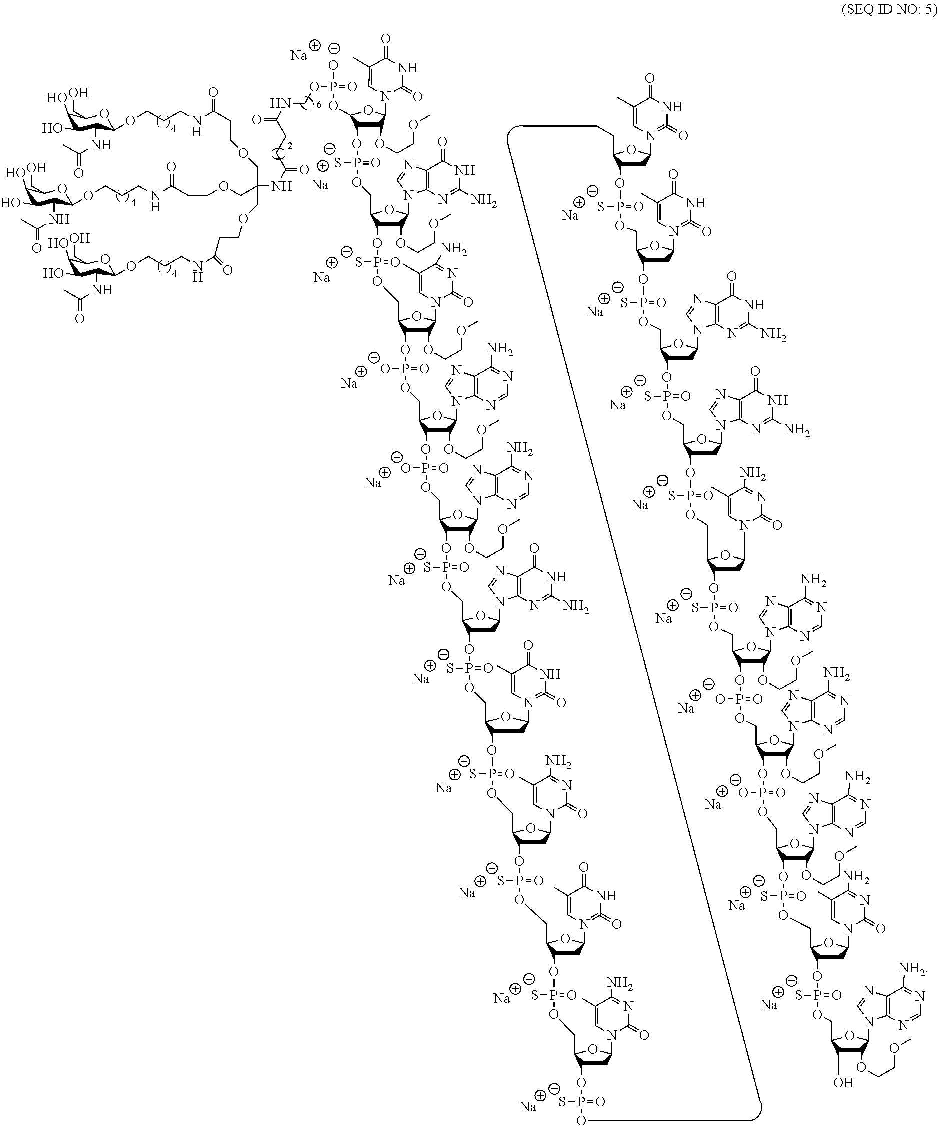

Embodiment 20. A method of reducing PKK activity in a human subject in need thereof, the method comprising administering to the human subject a therapeutically effective amount of an oligomeric compound, wherein the oligomeric compound has the following chemical notation (5′ to 3′): (THA-GalNAc3)o Tes Ges mCeo Aeo Aes Gds Tds mCds Tds mCds Tds Tds Gds Gds mCds Aeo Aeo Aes mCes Ae (SEQ ID NO: 5); wherein (THA-GalNAc3)o is represented by the following structure:

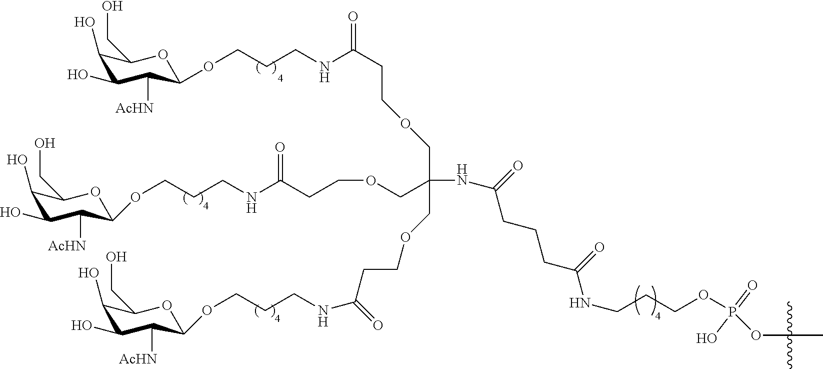

and wherein,

$$A = an adenine nucleobase ,$$

$$mC = a 5 - methyl cytosine nucleobase ,$$

$$G = a guanine nucleobase ,$$

$$T = a thymine nucleobase ,$$

$$e = a 2 ’ - MOE sugar moiety ,$$

$$d = a 2 ’ - a 2 ’ - β - D - deoxyribosyl sugar moiety ,$$

$$s = a phosphorothioate internucleoside linkage , and$$

$$o = a phosphodiester internucleoside linkage .$$

Embodiment 21. The method of any one of embodiments 1-20, wherein the therapeutically effective amount is 20 mg.

Embodiment 22. The method of any one of embodiments 1-20, wherein the therapeutically effective amount is 40 mg.

Embodiment 23. The method of any one of embodiments 1-20, wherein the therapeutically effective amount is 60 mg.

Embodiment 24. The method of any one of embodiments 1-20, wherein the therapeutically effective amount is 80 mg.

Embodiment 25. The method of any one of embodiments 1-20, wherein the therapeutically effective amount is 100 mg.

Embodiment 26. The method of any one of embodiments 1-20, wherein the therapeutically effective amount is any of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, and 300 mg.

Embodiment 27. The method of any one of embodiments 1-20, wherein the therapeutically effective amount is any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, and about 300 mg.

Embodiment 28. The method of any one of embodiments 1-20, wherein the therapeutically effective amount is any of 75.0 mg, 75.1 mg, 75.2 mg, 75.3 mg, 75.4 mg, 75.5 mg, 75.6 mg, 75.7 mg, 75.8 mg, 75.9 mg, 76.0 mg, 76.1 mg, 76.2 mg, 76.3 mg. 76.4 mg, 76.5 mg, 76.6 mg, 76.7 mg, 76.8 mg, 76.9 mg, 77.0 mg, 77.1 mg, 77.2 mg, 77.3 mg, 77.4 mg, 77.5 mg, 77.6 mg, 77.7 mg, 77.8 mg, 77.9 mg, 78.0 mg, 78.1 mg, 78.2 mg, 78.3 mg. 78.4 mg, 78.5 mg, 78.6 mg, 78.7 mg, 78.8 mg, 78.9 mg, 79.0 mg, 79.1 mg, 79.2 mg, 79.3 mg, 79.4 mg, 79.5 mg, 79.6 mg, 79.7 mg, 79.8 mg, 79.9 mg, 80.0 mg, 80.1 mg, 80.2 mg, 80.3 mg. 80.4 mg, 80.5 mg, 80.6 mg, 80.7 mg, 80.8 mg, 80.9 mg, 81.0 mg, 81.1 mg, 81.2 mg, 81.3 mg, 81.4 mg, 81.5 mg, 81.6 mg, 81.7 mg, 81.8 mg, 81.9 mg, 82.0 mg, 82.1 mg, 82.2 mg, 82.3 mg. 82.4 mg, 82.5 mg, 82.6 mg, 82.7 mg, 82.8 mg, 82.9 mg, 83.0 mg, 83.1 mg, 83.2 mg, 83.3 mg, 83.4 mg, 83.5 mg, 83.6 mg, 83.7 mg, 83.8 mg, 83.9 mg, 84.0 mg, 84.1 mg, 84.2 mg, 84.3 mg. 84.4 mg, 84.5 mg, 84.6 mg, 84.7 mg, 84.8 mg, 84.9 mg, and 85.0 mg.

Embodiment 29. The method of any one of embodiments 1-20, wherein the therapeutically effective amount is any of about 75.0 mg, about 75.1 mg, about 75.2 mg, about 75.3 mg, about 75.4 mg, about 75.5 mg, about 75.6 mg, about 75.7 mg, about 75.8 mg, about 75.9 mg, about 76.0 mg, about 76.1 mg, about 76.2 mg, about 76.3 mg, about 76.4 mg, about 76.5 mg, about 76.6 mg, about 76.6 about 76.7 mg, about 76.8 mg, about 76.9 mg, about 77.0 mg, about 77.1 mg, about 77.2 mg, about 77.3 mg, about 77.4 mg, about 77.5 mg, about 77.6 mg, about 77.7 mg, about 77.8 mg, about 77.9 mg, about 78.0 mg, about 78.1 mg, about 78.2 mg, about 78.3 mg, about 78.4 mg, about 78.5 mg, about 78.6 mg, about 78.7 mg, about 78.8 mg, about 78.9 mg, about 79.0 mg, about 79.1 mg, about 79.2 mg, about 79.3 mg, about 79.4 mg, about 79.5 mg, about 79.6 mg, about 79.7 mg, about 79.8 mg, about 79.9 mg, about 80.0 mg, about 80.1 mg, about 80.2 mg, about 80.3 mg, about 80.4 mg, about 80.5 mg, about 80.6 mg, about 80.7 mg, about 80.8 mg, about 80.9 mg, about 81.0 mg, about 81.1 mg, about 81.2 mg, about 81.3 mg, about 81.4 mg, about 81.5 mg, about 81.6 mg, about 81.7 mg, about 81.8 mg, about 81.9 mg, about 82.0 mg, about 82.1 mg, about 82.2 mg, about 82.3 mg, about 82.4 mg, about 82.5 mg, about 82.6 mg, about 82.7 mg, about 82.8 mg, about 82.9 mg, about 83.0 mg, about 83.1 mg, about 83.2 mg, about 83.3 mg, about 83.4 mg, about 83.5 mg, about 83.6 mg, about 83.7 mg, about 83.8 mg, about 83.9 mg, about 84.0 mg, about 84.1 mg, about 84.2 mg, about 84.3 mg, about 84.4 mg, about 84.5 mg, about 84.6 mg, about 84.7 mg, about 84.8 mg, about 84.9 mg, and about 85.0 mg.

Embodiment 30. The method of any one of embodiments 1-20, wherein the therapeutically effective amount is any of 10 mg to 140 mg, 10 mg to 130 mg, 10 mg to 120 mg, 10 mg to 110 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 140 mg, 20 mg to 130 mg, 20 mg to 120 mg, 20 mg to 110 mg, 20 mg to 100 mg, 20 mg to 90 mg, 20 mg to 80 mg, 20 mg to 70 mg, 20 mg to 60 mg, 20 mg to 50 mg, 20 mg to 40 mg, 20 mg to 30 mg, 30 mg to 140 mg, 30 mg to 130 mg, 30 mg to 120 mg, 30 mg to 110 mg, 30 mg to 100 mg, 30 mg to 90 mg, 30 mg to 80 mg, 30 mg to 70 mg, 30 mg to 60 mg, 30 mg to 50 mg, 30 mg to 40 mg, 40 mg to 140 mg, 40 mg to 130 mg, 40 mg to 120 mg, 40 mg to 110 mg, 40 mg to 100 mg, 40 mg to 90 mg, 40 mg to 80 mg, 40 mg to 70 mg, 40 mg to 60 mg, 40 mg to 50 mg, 50 mg to 140 mg, 50 mg to 130 mg, 50 mg to 120 mg, 50 mg to 110 mg, 50 mg to 100 mg, 50 mg to 90 mg, 50 mg to 80 mg, 50 mg to 70 mg, 50 mg to 60 mg, 60 mg to 140 mg, 60 mg to 130 mg, 60 mg to 120 mg, 60 mg to 110 mg, 60 mg to 100 mg, 60 mg to 90 mg, 60 mg to 80 mg, 60 mg to 70 mg, 70 mg to 140 mg, 70 mg to 130 mg, 70 mg to 120 mg, 70 mg to 110 mg, 70 mg to 100 mg, 70 mg to 90 mg, 70 mg to 80 mg, 80 mg to 140 mg, 80 mg to 130 mg, 80 mg to 120 mg, 80 mg to 110 mg, 80 mg to 100 mg, 80 mg to 90 mg, 90 mg to 140 mg, 90 mg to 130 mg, 90 mg to 120 mg, 90 mg to 110 mg, 90 mg to 100 mg, 100 mg to 140 mg, 100 mg to 130 mg, 100 mg to 120 mg, 100 mg to 110 mg, 110 mg to 140 mg, 110 mg to 130 mg, 110 mg to 120 mg, 120 mg to 140 mg, 120 mg to 130 mg, 130 mg to 140 mg, 65 mg to 95 mg, 65 mg to 90 mg, 65 mg to 85 mg 65 mg to 80 mg, 65 mg to 75 mg, 65 mg to 70 mg, 70 mg to 95 mg, 70 mg to 85 mg, 70 mg to 75 mg, 75 mg to 100 mg, 75 mg to 95 mg, 75 mg to 90 mg, 75 mg to 85 mg, 75 mg to 80 mg, 80 mg to 95 mg, 80 mg to 85 mg, 85 mg to 100 mg, 85 mg to 90 mg, 90 mg to 95 mg, 95 mg to 100 mg, 80 mg to 89 mg, 80 mg to 88 mg, 80 mg to 87 mg, 80 mg to 86 mg, 80 mg to 84 mg, 80 mg to 83 mg, 80 mg to 82 mg, 80 mg to 81 mg, 81 mg to 90 mg, 82 mg to 89 mg, 82 mg to 88 mg, 82 mg to 87 mg, 82 mg to 86 mg, 82 mg to 85 mg, 82 mg to 84 mg, 82 mg to 83 mg, 83 mg to 90 mg, 83 mg to 89 mg, 83 mg to 88 mg, 83 mg to 87 mg, 83 mg to 86 mg, 83 mg to 85 mg, 83 mg to 84 mg, 84 mg to 90 mg, 84 mg to 89 mg, 84 mg to 88 mg, 84 mg to 87 mg, 84 mg to 86 mg, 84 mg to 85 mg, 85 mg to 89 mg, 85 mg to 88 mg, 85 mg to 87 mg, 85 mg to 86 mg, 86 mg to 90 mg, 86 mg to 89 mg, 86 mg to 88 mg, 86 mg to 87 mg, 87 mg to 90 mg, 87 mg to 89 mg, 87 mg to 88 mg, 88 mg to 90 mg, 88 mg to 89 mg, and 89 mg to 90 mg.

Embodiment 31. The method of any one of embodiments 1-20, wherein the therapeutically effective amount is any of less than 300 mg, less than 295 mg, less than 290 mg, less than 285 mg, less than 280 mg, less than 275 mg, less than 270 mg, less than 265 mg, less than 260 mg, less than 255 mg, less than 250 mg, less than 245 mg, less than 240 mg, less than 235 mg, less than 230 mg, less than 225 mg, less than 220 mg, less than 215 mg, less than 210 mg, less than 205 mg, less than 200 mg, less than 195 mg, less than 190 mg, less than 185 mg, less than 180 mg, less than 175 mg, less than 170 mg, less than 165 mg, less than 160 mg, less than 150 mg, less than 145 mg, less than 140 mg, less than 135 mg, less than 130 mg, less than 125 mg, less than 120 mg, less than 115 mg, less than 110 mg, less than 105 mg, less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, less than 40 mg, less than 35 mg, less than 30 mg, less than 25 mg, and less than 20 mg.

Embodiment 32. The method of any one of embodiments 1-20, wherein the therapeutically effective amount is any of less than about 300 mg, less than about 295 mg, less than about 290 mg, less than about 285 mg, less than about 280 mg, less than about 275 mg, less than about 270 mg, less than about 265 mg, less than about 260 mg, less than about 255 mg, less than about 250 mg, less than about 245 mg, less than about 240 mg, less than about 235 mg, less than about 230 mg, less than about 225 mg, less than about 220 mg, less than about 215 mg, less than about 210 mg, less than about 205 mg, less than about 200 mg, less than about 195 mg, less than about 190 mg, less than about 185 mg, less than about 180 mg, less than about 175 mg, less than about 170 mg, less than about 165 mg, less than about 160 mg, less than about 150 mg, less than about 145 mg, less than about 140 mg, less than about 135 mg, less than about 130 mg, less than about 125 mg, less than about 120 mg, less than about 115 mg, less than about 110 mg, less than about 105 mg, less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, less than about 40 mg, less than about 35 mg, less than about 30 mg, less than about 25 mg, and less than about 20 mg.

Embodiment 33. The method of any one of embodiments 1-20, wherein the therapeutically effective amount is any of at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least about 100 mg, at least 105 mg, at least 115 mg, at least 120 mg, at least 125 mg, at least 130 mg, at least 135 mg, at least 140 mg, at least 145 mg, and at least 150 mg.

Embodiment 34. The method of any one of embodiments 1-20, wherein the therapeutically effective amount is any of at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 115 mg, at least about 120 mg, at least about 125 mg, at least about 130 mg, at least about 135 mg, at least about 140 mg, at least about 145 mg, and at least about 150 mg.

Embodiment 35. The method of any one of embodiments 1-34, comprising administering the oligomeric compound once every 4 weeks.

Embodiment 36. The method of any one of embodiments 1-34, comprising administering the oligomeric compound once every 8 weeks.

Embodiment 37. The method of any one of embodiments 1-34, comprising administering the oligomeric compound once about every 4 weeks.

Embodiment 38. The method of any one of embodiments 1-34, comprising administering the oligomeric compound once about every 8 weeks.

Embodiment 39. The method of any one of embodiments 1-34, comprising administering the oligomeric compound once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, or once every 20 weeks.

Embodiment 40. The method of any one of embodiments 1-34, comprising administering the oligomeric compound once about every 4 weeks, once about every 5 weeks, once about every 6 weeks, once about every 7 weeks, once about every 8 weeks, once about every 9 weeks, once about every 10 weeks, once about every 11 weeks, once about every 12 weeks, once about every 13 weeks, once about every 14 weeks, once about every 15 weeks, once about every 16 weeks, once about every 17 weeks, once about every 18 weeks, once about every 19 weeks, or once about every 20 weeks.

Embodiment 41. The method of any one of embodiments 1-34, comprising administering at least two loading doses of the oligomeric compound, and at least two maintenance doses of the oligomeric compound.

Embodiment 42. The method of embodiment 41, wherein the loading doses are an amount of 80 mg or about 80 mg.

Embodiment 43. The method of embodiment 41 or 42, wherein the maintenance doses are an amount less than 80 mg.

Embodiment 44. The method of embodiment 43, wherein the maintenance dose is 20 mg, 25 mg, 30 mg, 35 mg, 40 mg, 45 mg, 50 mg, 55 mg, 60 mg, 65 mg, 70 mg, or 75 mg.

Embodiment 45. The method of embodiment 43, wherein the maintenance dose is about 20 mg, about 25 mg, about 30 mg, about 35 mg, about 40 mg, about 45 mg, about 50 mg, about 55 mg, about 60 mg, about 65 mg, about 70 mg, or about 75 mg.

Embodiment 46. The method of any one of embodiments 41-45, comprising administering the loading doses once every 4 weeks, and administering at least two maintenance doses once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, or once every 20 weeks.

Embodiment 47. The method of any one of embodiments 41-45, comprising administering the loading doses once about every 4 weeks, and administering at least two maintenance doses once about every 5 weeks, once about every 6 weeks, once about every 7 weeks, once about every 8 weeks, once about every 9 weeks, once about every 10 weeks, once about every 11 weeks, once about every 12 weeks, once about every 13 weeks, once about every 14 weeks, once about every 15 weeks, once about every 16 weeks, once about every 17 weeks, once about every 18 weeks, once about every 19 weeks, or once about every 20 weeks.

Embodiment 48. The method of any one of embodiments 41-45, comprising administering the loading doses once every 2 weeks, and administering at least two maintenance doses once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, or once every 20 weeks.

Embodiment 49. The method of any one of embodiments 41-45, comprising administering the loading doses once about every 2 weeks, and administering at least two maintenance doses once about every 5 weeks, once about every 6 weeks, once about every 7 weeks, once about every 8 weeks, once about every 9 weeks, once about every 10 weeks, once about every 11 weeks, once about every 12 weeks, once about every 13 weeks, once about every 14 weeks, once about every 15 weeks, once about every 16 weeks, once about every 17 weeks, once about every 18 weeks, once about every 19 weeks, or once about every 20 weeks.

Embodiment 50. The method of any one of embodiments 1-49, wherein the human subject has hereditary angioedema or a risk thereof.

Embodiment 51. The method of embodiment 50, wherein the hereditary angioedema is type I HAE.

Embodiment 52. The method of embodiment 50, wherein the hereditary angioedema is type II HAE.

Embodiment 53. The method of embodiment 51 or 52, wherein the human subject has a genetic mutation in the SERPING1 gene.

Embodiment 54. The method of embodiment 50, wherein the hereditary angioedema is type III HAE.

Embodiment 55. The method of embodiment 54, wherein the human subject has a genetic mutation in a gene selected from F12, PLG, and ANGPT1.

Embodiment 56. The method of embodiment 54, wherein the subject does not have a genetic mutation in a gene selected from F12, PLG, and ANGPT1.

Embodiment 57. The method of any one of embodiments 50-56, wherein the human subject is refractory to at least one anti-edema agent.

Embodiment 58. The method of embodiment 57, wherein the at least one anti-edema agent comprises a histamine inhibitor, a C1 esterase inhibitor, attenuated androgen, an antifibrinolytic agent, an angiotensin-converting enzyme inhibitor, an angiotensin II type 1 receptor blocker, a kallikrein inhibitor, a bradykinin B2 receptor antagonist, or a combination thereof.

Embodiment 59. The method of any one of embodiments 50-58, wherein the human subject is refractory to a histamine inhibitor, an antifibrinolytic agent, an attenuated androgen, a C1 esterase inhibitor, and a bradykinin B2 receptor antagonist.

Embodiment 60. The method of embodiment 58 or 59, wherein the antifibrinolytic agent is tranexamic acid.

Embodiment 61. The method of any one of embodiments 58-60, wherein the attenuated androgen is danazol.

Embodiment 62. The method of any one of embodiments 58-61, wherein the bradykinin B2 receptor antagonist is icatibant.

Embodiment 63. The method of any one of embodiments 57-62, wherein the at least one anti-edema agent provides less than about 5%, less than about 10%, less than about 20%, less than about 30%, less than about 40%, or less than about 50% reduction in the occurrence of angioedema attacks experienced by the subject as measured over at least 1, at least 2, at least 3, at least 4 or at least 6 months after an initial administration of the at least one anti-edema agent, relative to the occurrence of angioedema attacks experienced by the subject before the initial administration of the at least one anti-edema agent.

Embodiment 64. The method of any one of embodiments 57-63, wherein the at least one anti-edema agent provides less than about 5%, less than about 10%, less than about 20%, less than about 30%, less than about 40%, or less than about 50% reduction in swelling about 1 hour, about 2 hours, about 4 hours, about 8 hours, about 12 hours, about 24, or about 48 hours after an initial administration of the at least one anti-edema agent.

Embodiment 65. The method of any one of embodiments 50-64, wherein the human subject has an average of at least 1, at least 2, at least 3, at least 4, at least 5, or at least 6 angioedema attacks per month, as measured over at least 1, 2, 3, 4, 5, 6, 8, or 12 months, before the administering.

Embodiment 66. The method of any one of embodiments 1-49, wherein the human subject has macular edema or a risk thereof.

Embodiment 67. The method of embodiment 66, wherein the human subject has diabetes, diabetic macular edema, diabetic retinopathy, or a combination thereof.

Embodiment 68. A method of ameliorating hereditary angioedema in a human subject in need thereof, the method comprising administering to the human subject about 80 mg of an oligomeric compound according to the following chemical structure:

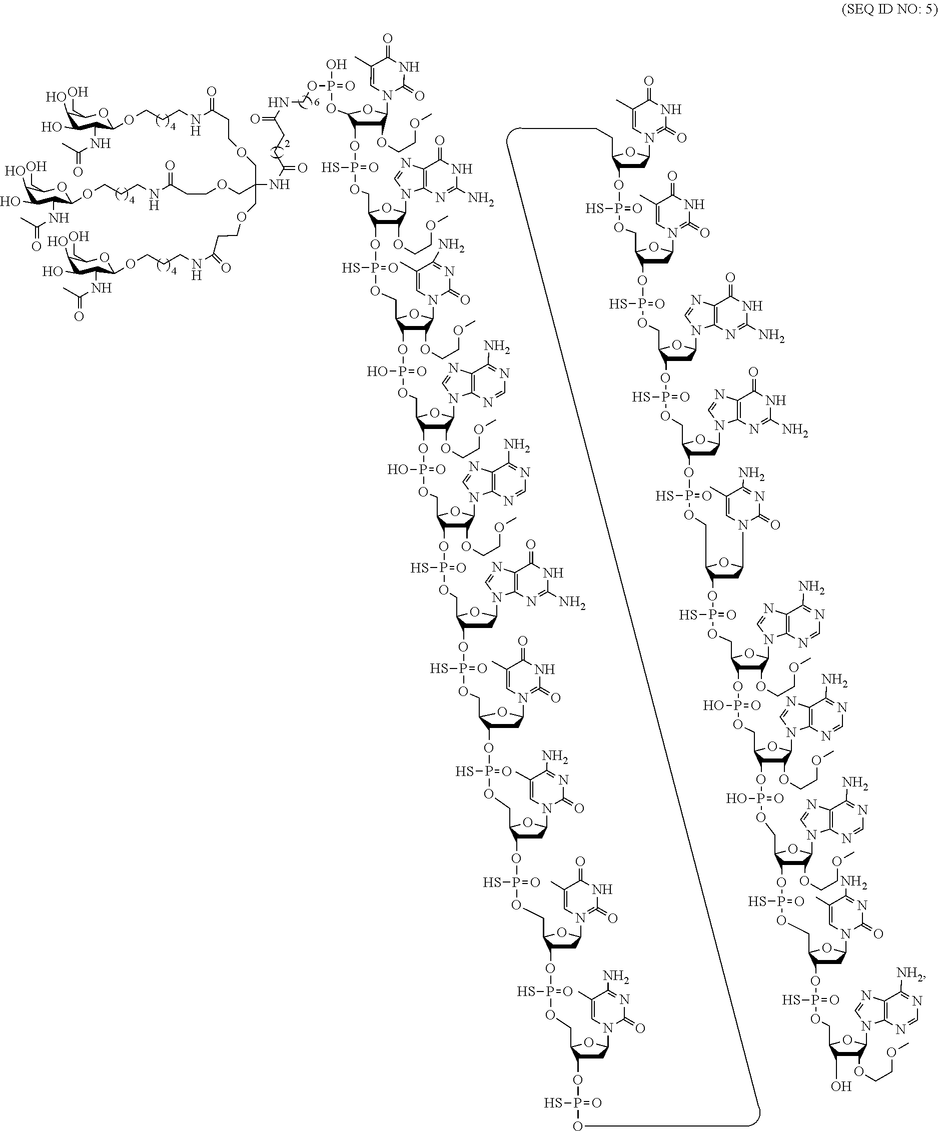

or a salt thereof.

Embodiment 69. The method of embodiment 68, wherein the salt is the sodium salt or the potassium salt.

Embodiment 70. A method of ameliorating hereditary angioedema in a human subject in need thereof, the method comprising administering to the human subject about 80 mg of an oligomeric compound according to the following chemical structure:

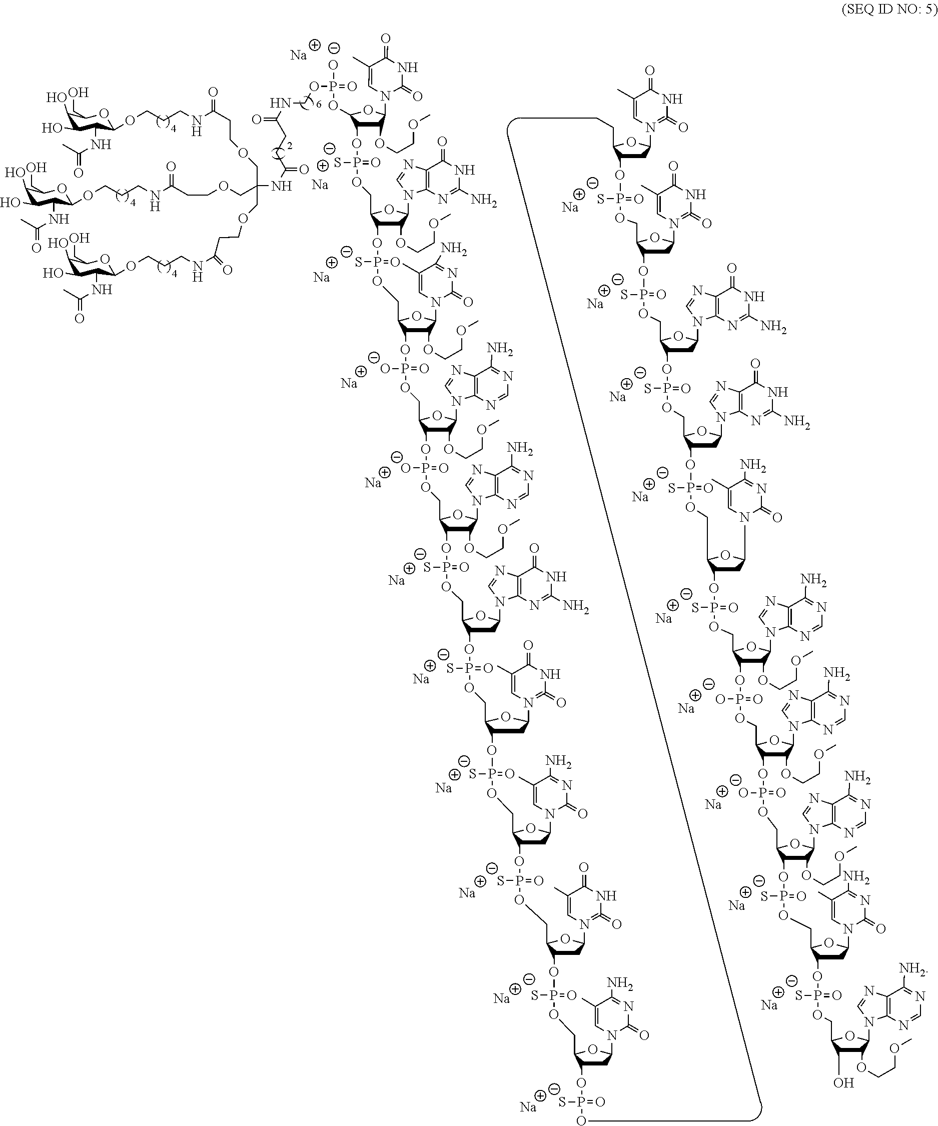

Embodiment 71. A method of ameliorating hereditary angioedema in a human subject in need thereof, the method comprising administering to the human subject about 80 mg of an oligomeric compound, wherein the oligomeric compound has the following chemical notation (5′ to 3′): (THA-GalNAc3)o Tes Ges mCeo Aeo Aes Gds Tds mCds Tds mCds Tds Tds Gds Gds mCds Aeo Aeo Aes mCes Ae (SEQ ID NO: 5); wherein (THA-GalNAc3)o is represented by the following structure:

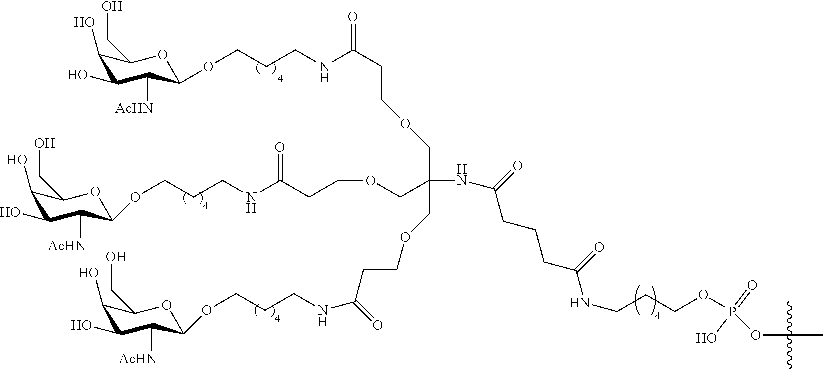

and wherein,

$$A = an adenine nucleobase ,$$

$$mC = a 5 - methyl cytosine nucleobase ,$$

$$G = a guanine nucleobase ,$$

$$T = a thymine nucleobase ,$$

$$e = a 2 ’ - MOE sugar moiety ,$$

$$d = a 2 ’ - a 2 ’ - β - D - deoxyribosyl sugar moiety ,$$

$$s = a phosphorothioate internucleoside linkage , and$$

$$o = a phosphodiester internucleoside linkage .$$

Embodiment 72. The method of any one of embodiments 68-71, comprising administering to the human subject 80 mg of the oligomeric compound.

Embodiment 73. The method of any one of embodiments 68-71, wherein the method comprises administering to the human subject at least two doses of about 80 mg of the oligomeric compound once about every 4 weeks, and subsequently administering at least two doses of about 100 mg of the oligomeric compound once about every 4 weeks.

Embodiment 74. The method of any one of embodiments 68-71, wherein the method comprises administering to the human subject at least two doses of 80 mg of the oligomeric compound once every 4 weeks, and subsequently administering at least two doses of 100 mg of the oligomeric compound once every 4 weeks.

Embodiment 75. The method of embodiment 73 or 74, wherein the human subject has at least one angioedema attack during the administering of 80 mg or about 80 mg of the oligomeric compound once every 4 weeks or once about every 4 weeks.

Embodiment 76. The method of any one of embodiments 68-71, wherein the method comprises administering to the human subject at least two loading doses of about 80 mg of the oligomeric compound about every 4 weeks, and at least two maintenance doses of about 80 mg of the oligomeric compound about every 8 weeks.

Embodiment 77. The method of any one of embodiments 68-71, wherein the method comprises administering to the human subject at least two loading doses of 80 mg of the oligomeric compound once every 4 weeks, and at least two maintenance doses of 80 mg of the oligomeric compound once every 8 weeks.

Embodiment 78. The method of any one of embodiments 75-77, wherein the subject does not have an angioedema attack during the administering of 80 mg or about 80 mg of the oligomeric compound once every 4 weeks or once about every 4 weeks

Embodiment 79. The method any one of embodiments 68-78, wherein the hereditary angioedema is type I HAE, type II HAE, or type III HAE.

Embodiment 80. The method of embodiment 79, wherein the hereditary angioedema is type III HAE, and the human subject has a genetic mutation in a gene selected from F12, PLG, and ANGPT1.

Embodiment 81. The method of embodiment 79, wherein the hereditary angioedema is type III HAE, and the human subject does not have a genetic mutation in a gene selected from F12, PLG, and ANGPT1.

Embodiment 82. The method of any one of embodiments 68-81, wherein the human subject is refractory to at least one anti-edema agent.

Embodiment 83. The method of embodiment 82, wherein the at least one anti-edema agent comprises a histamine inhibitor, a C1 esterase inhibitor, attenuated androgen, an antifibrinolytic agent, an angiotensin-converting enzyme inhibitor, an angiotensin II type 1 receptor blocker, a kallikrein inhibitor, a bradykinin B2 receptor antagonist, or a combination thereof.

Embodiment 84. The method of any one of embodiments 68-83, wherein the human subject is refractory to a histamine inhibitor, an antifibrinolytic agent, an attenuated androgen, a C1 esterase inhibitor, and a bradykinin B2 receptor antagonist.

Embodiment 85. The method of embodiment 83 or 84, wherein the antifibrinolytic agent is tranexamic acid.

Embodiment 86. The method of any one of embodiments 83-85, wherein the attenuated androgen is danazol.

Embodiment 87. The method of any one of embodiments 83-86, wherein the bradykinin B2 receptor antagonist is icatibant.

Embodiment 88. The method of any one of embodiments 82-87, wherein the at least one anti-edema agent provides less than about 5%, less than about 10%, less than about 20%, less than about 30%, less than about 40%, or less than about 50% reduction in the occurrence of angioedema attacks experienced by the subject as measured over at least 1, at least 2, at least 3, at least 4 or at least 6 months after an initial administration of the at least one anti-edema agent, relative to the occurrence of angioedema attacks experienced by the subject before the initial administration of the at least one anti-edema agent.

Embodiment 89. The method of any one of embodiments 82-87, wherein the at least one anti-edema agent provides less than about 5%, less than about 10%, less than about 20%, less than about 30%, less than about 40%, or less than about 50% reduction in swelling about 1 hour, about 2 hours, about 4 hours, about 8 hours, about 12 hours, about 24, or about 48 hours after an initial administration of the at least one anti-edema agent.

Embodiment 90. The method of any one of embodiments 82-87, wherein the human subject has an average of at least 1, at least 2, at least 3, at least 4, at least 5, or at least 6 angioedema attacks per month as measured over at least 1, 2, 3, 4, 5, 6, 8, or 12 months, before the administering.

Embodiment 91. A method of ameliorating macular edema in a human subject in need thereof, the method comprising administering to the human subject about 80 mg of an oligomeric compound according to the following chemical structure:

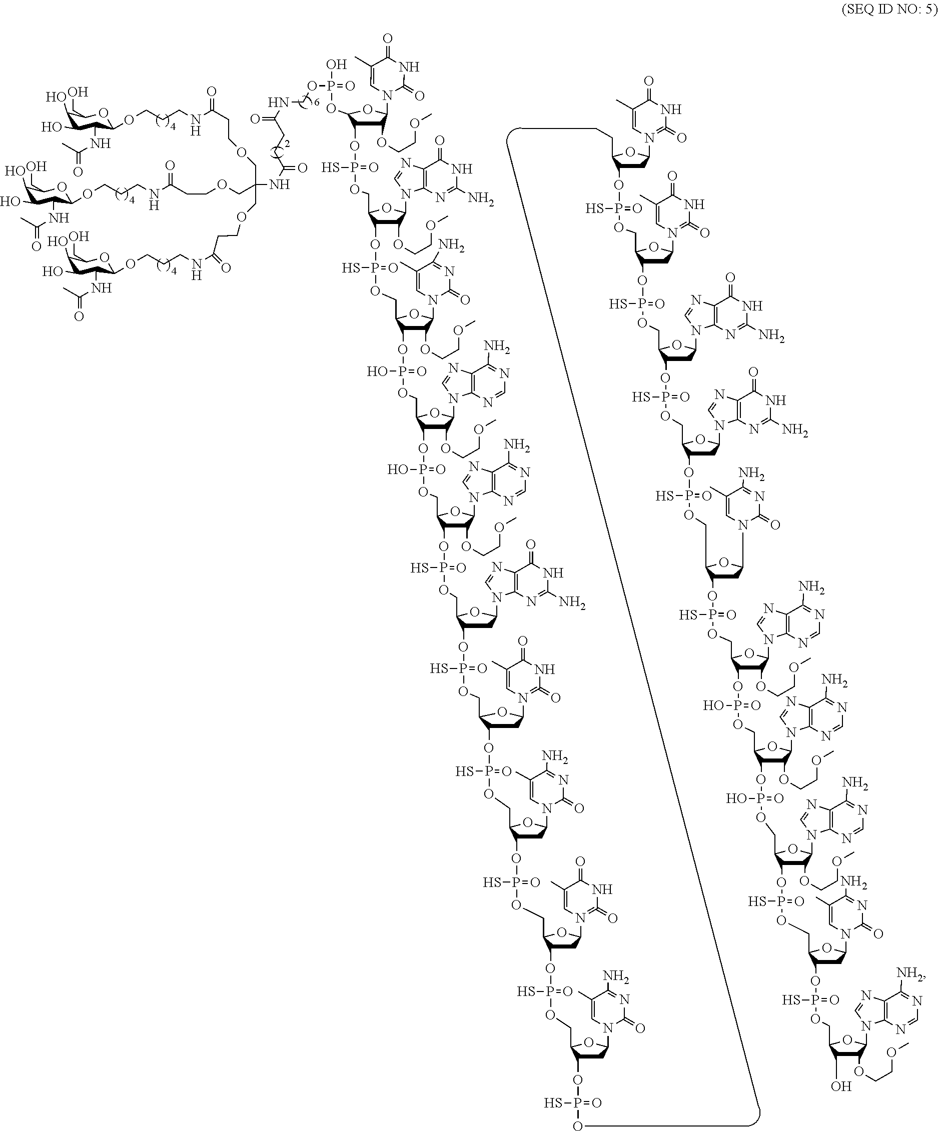

or a salt thereof.

Embodiment 92. The method of embodiment 91, wherein the salt is the sodium salt or the potassium salt.

Embodiment 93. A method of ameliorating macular edema in a human subject in need thereof, the method comprising administering to the human subject about 80 mg of an oligomeric compound according to the following chemical structure:

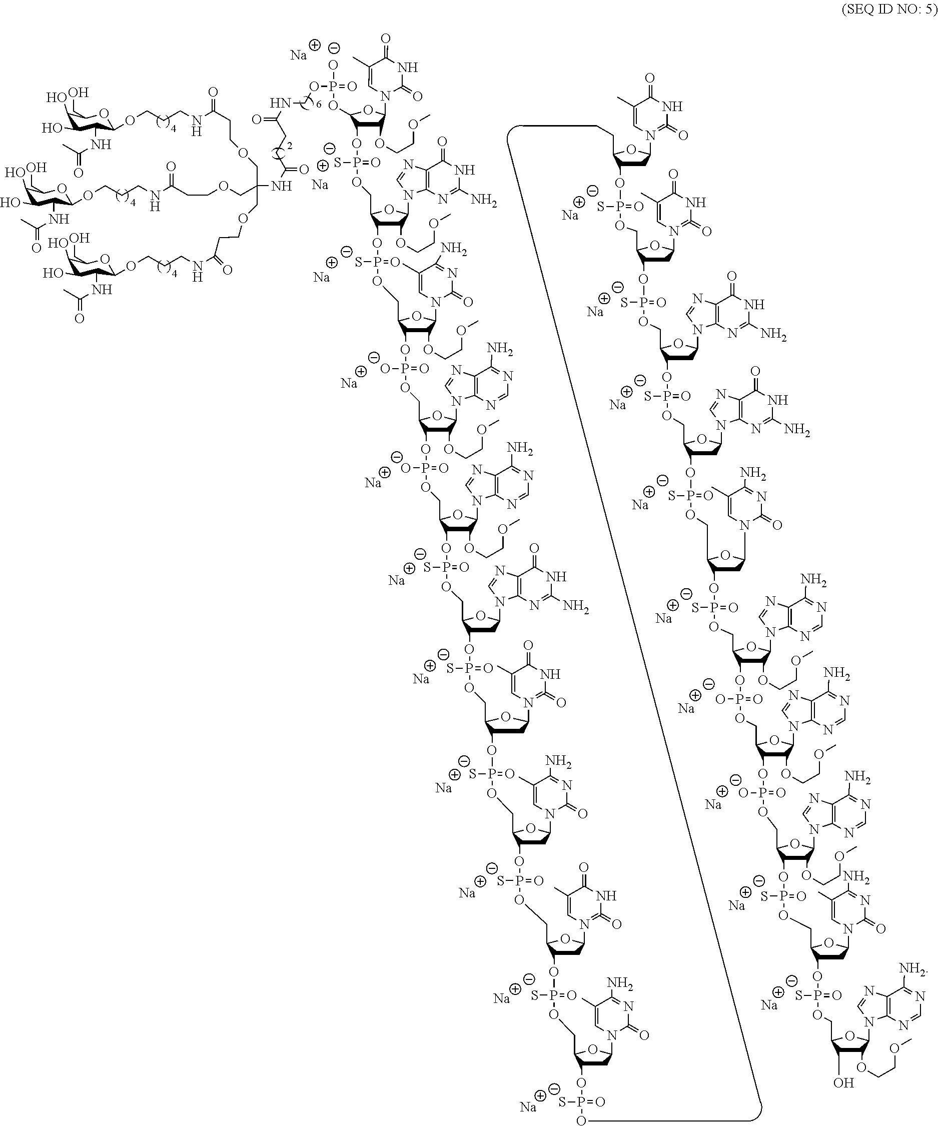

Embodiment 94. A method of ameliorating macular edema in a human subject in need thereof, the method comprising administering to the human subject about 80 mg of an oligomeric compound, wherein the oligomeric compound has the following chemical notation (5′ to 3′): (THA-GalNAc3)o Tes Ges mCeo Aeo Aes Gds Tds mCds Tds mCds Tds Tds Gds Gds mCds Aeo Aeo Aes mCes Ae (SEQ ID NO: 5); wherein (THA-GalNAc3)o is represented by the following structure:

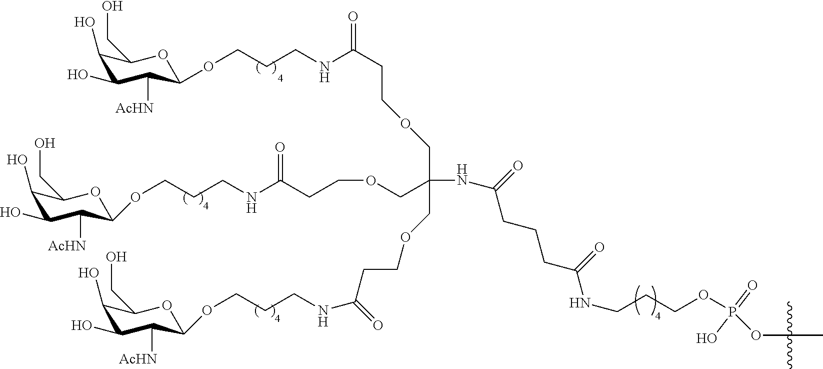

and wherein,

$$A = an adenine nucleobase ,$$

$$mC = a 5 - methyl cytosine nucleobase ,$$

$$G = a guanine nucleobase ,$$

$$T = a thymine nucleobase ,$$

$$e = a 2 ’ - MOE sugar moiety ,$$

$$d = a 2 ’ - a 2 ’ - β - D - deoxyribosyl sugar moiety ,$$

$$s = a phosphorothioate internucleoside linkage , and$$

$$o = a phosphodiester internucleoside linkage .$$

Embodiment 95. The method of any one of embodiments 91-94, comprising administering to the human subject 80 mg of the oligomeric compound.

Embodiment 96. The method of any one of embodiments 91-95, comprising administering to the human subject at least two loading doses of about 80 mg of the oligomeric compound once about every 2 weeks, and at least two maintenance doses of about 80 mg of the oligomeric compound once about every 4 weeks.

Embodiment 97. The method of any one of embodiments 91-95, comprising administering to the human subject at least two loading doses of 80 mg of the oligomeric compound once every 2 weeks, and at least two maintenance doses of 80 mg of the oligomeric compound once every 4 weeks.

Embodiment 98. The method of any one of embodiments 91-97, wherein the human subject has diabetic macular edema.

Embodiment 99. The method of any one of embodiments 1-98, wherein the human subject has an inflammatory condition, a thromboembolic condition, edema, or a risk thereof.

Embodiment 100. The method of any of embodiments 1-99, wherein the oligomeric compound is administered by subcutaneous injection.

Embodiment 101. The method of any one of embodiments 1-100, wherein the oligomeric compound is self-administered.

Embodiment 102. The method of any one of embodiments 1-101, wherein PKK RNA is reduced in the subject.

Embodiment 103. The method of any one of embodiments 1-102, wherein PKK protein is reduced in the subject.

Embodiment 104. The method of any one of embodiments 1-103, wherein PKK activity is reduced in the subject.

### I. PKK

In certain embodiments, described herein are methods of reducing PKK RNA and/or PKK protein in a cell or a biological fluid of a subject. PKK RNA is encoded by the human KLKB1 gene, located on human chromosome 4. PKK protein is the protein expression product of PKK RNA. PKK protein is highly expressed in liver relative to other cell types. A representative nucleobase sequence for the human KLKB1 gene is provided at GENBANK Accession No. NT_016354.19 truncated from nucleotides 111693001 to 111730000, incorporated herein as SEQ ID NO: 1. A representative nucleobase sequence for a human PKK mRNA is provided at GENBANK Accession No. NM_000892.3, incorporated herein as SEQ ID NO: 2. A representative protein sequence for a human PKK protein is provided at GENBANK Accession No. NP_000883.2, incorporated here in as SEQ ID NO: 3.

### II. ISIS 721744

In certain embodiments, described herein are methods of administering oligomeric compound, ISIS 721744, to a subject in need thereof. In certain embodiments, ISIS 721744 is characterized a 5-10-5 MOE gapmer, having a sequence of (from 5′ to 3′) TGCAAGTCTCTTGGCAAACA (incorporated herein as SEQ ID NO: 4), wherein each of nucleosides 1-5 and 16-20 (from 5′ to 3′) comprise a 2′-MOE modification and each of nucleosides 6-15 are 2′-deoxyribonucleosides, wherein the internucleoside linkages between nucleosides 3 to 4, 4 to 5, 16 to 17, and 17 to 18 are phosphodiester internucleoside linkages and the internucleoside linkages between nucleosides 1 to 2, 2 to 3, 5 to 6, 6 to 7, 7 to 8, 8 to 9, 9 to 10, 10 to 11, 11 to 12, 12 to 13, 13 to 14, 14 to 15, 15 to 16, 18 to 19, and 19 to 20 are phosphorothioate internucleoside linkages, and wherein each cytosine is a 5′-methyl cytosine.

In certain embodiments, ISIS 721744 is represented by the following chemical notation (5′ to 3′): (THA-GalNAc3)o Tes Ges mCeo Aeo Aes Gds Tds mCds Tds mCds Tds Tds Gds Gds mCds Aeo Aeo Aes mCes Ae (SEQ ID NO: 5); wherein (THA-GalNAc3)o is represented by the following structure:

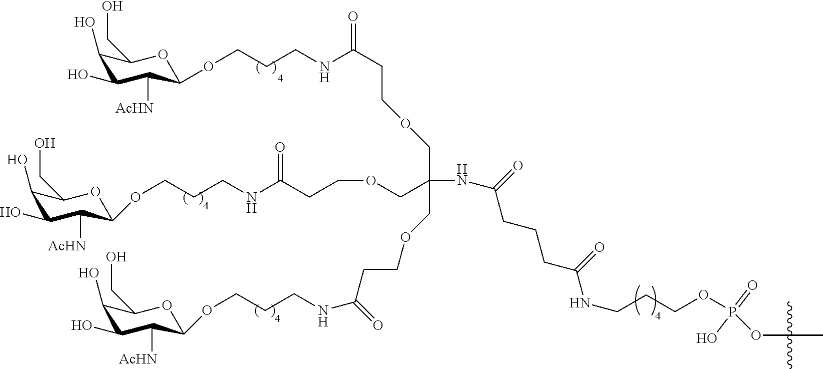

and wherein,

$$A = an adenine nucleobase ,$$

$$mC = a 5 - methyl cytosine nucleobase ,$$

$$G = a guanine nucleobase ,$$

$$T = a thymine nucleobase ,$$

$$e = a 2 ’ - MOE sugar moiety ,$$

$$d = a 2 ’ - a 2 ’ - β - D - deoxyribosyl sugar moiety ,$$

$$s = a phosphorothioate internucleoside linkage , and$$

$$o = a phosphodiester internucleoside linkage .$$

In certain embodiments, ISIS 721744 is represented by the following chemical structure:

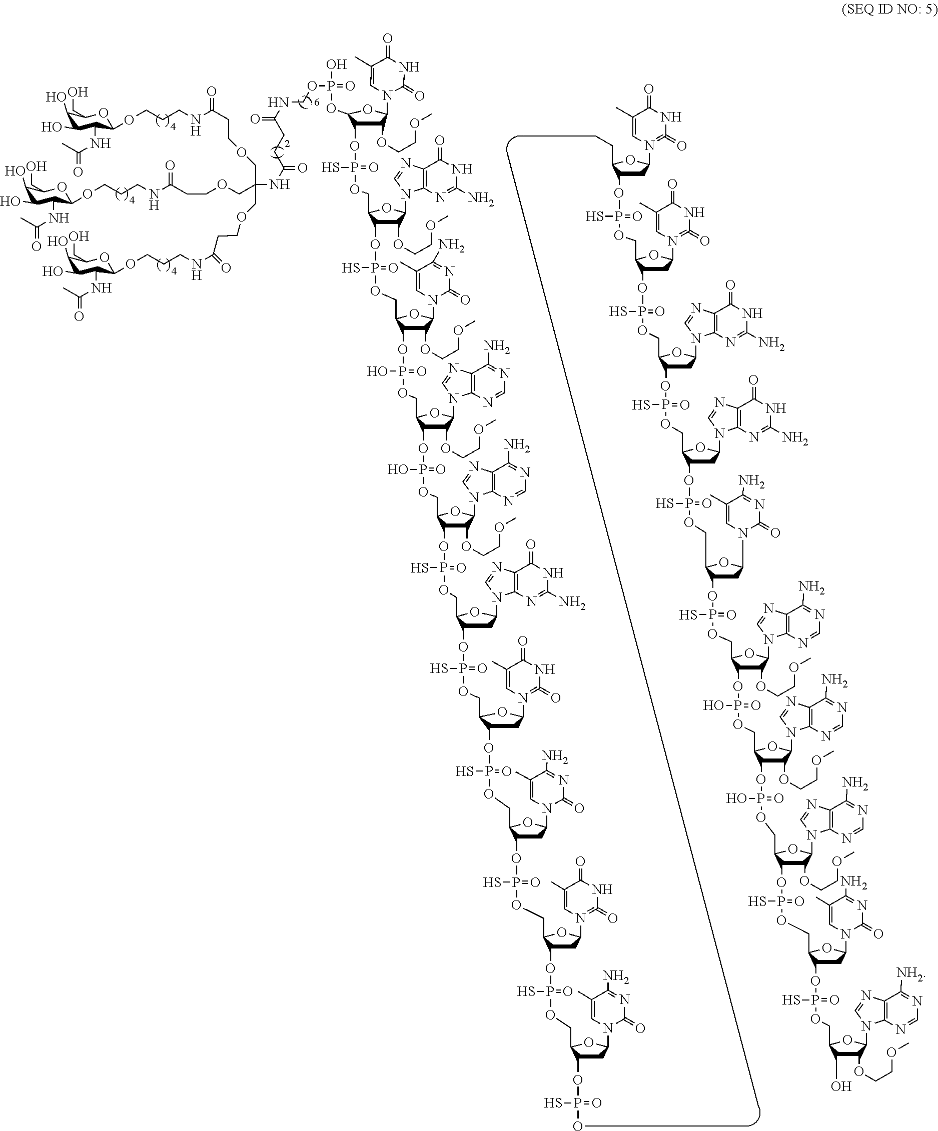

### Structure 1. ISIS 721744

In certain embodiments, the sodium salt of ISIS 721744 is represented by the following chemical structure:

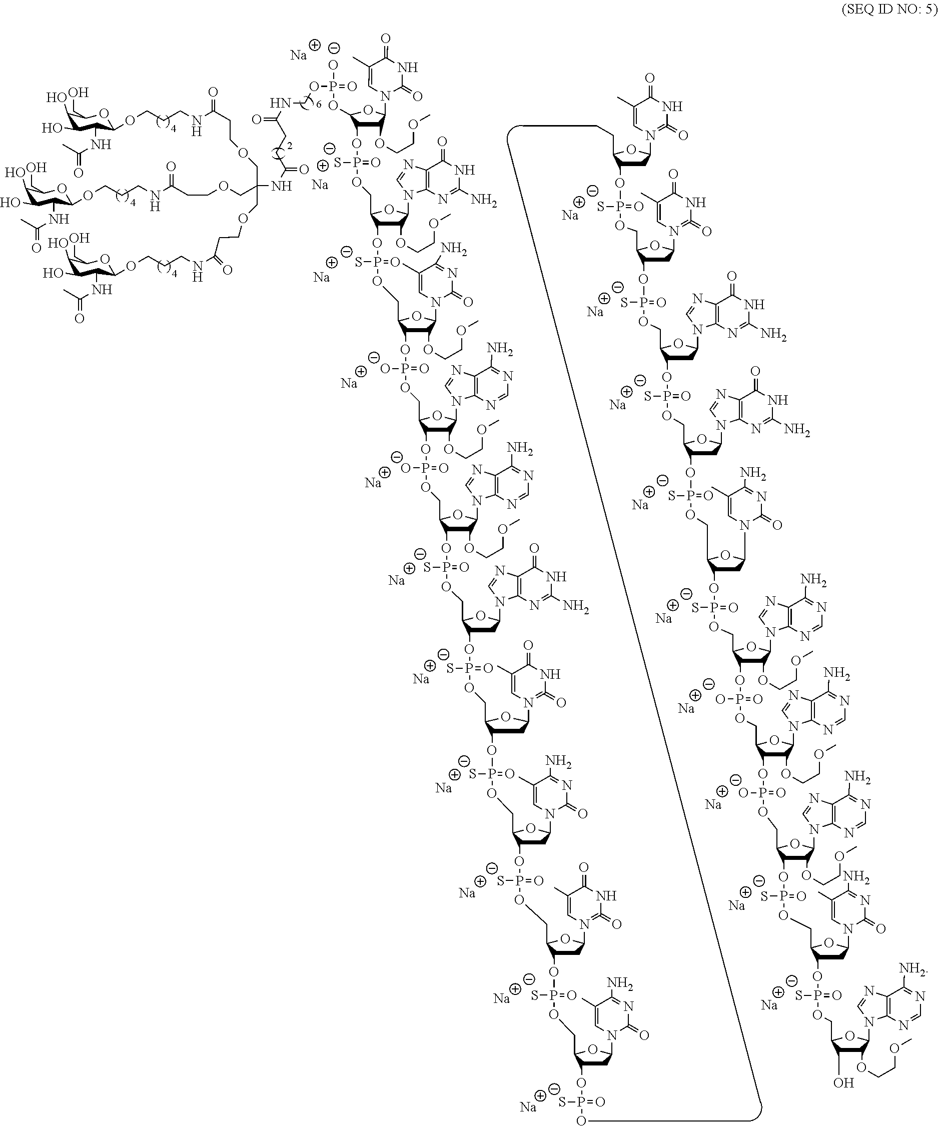

Structure 2. Sodium salt of ISIS 721744

### III. Certain Pharmaceutical Compositions

In certain embodiments, described herein are methods of administering to a subject a pharmaceutical composition comprising the oligomeric compound ISIS 721744. In certain embodiments, the pharmaceutical composition comprises a pharmaceutically acceptable diluent or carrier. In certain embodiments, the pharmaceutical composition comprises or consists essentially of a sterile saline solution and ISIS 721744. In certain embodiments, the sterile saline is pharmaceutical grade saline. In certain embodiments, the pharmaceutical composition comprises or consists essentially of sterile water and ISIS 721744. In certain embodiments, the sterile water is pharmaceutical grade water. In certain embodiments, the pharmaceutical composition comprises or consists essentially of phosphate buffered saline and ISIS 721744.

In certain embodiments, pharmaceutical compositions comprise one or more excipients and ISIS 721744. In certain embodiments, excipients are selected from water, salt solutions, alcohol, polyethylene glycols, gelatin, lactose, amylase, magnesium stearate, tale, silicic acid, viscous paraffin, hydroxymethylcellulose, and polyvinylpyrrolidone.

In certain embodiments, pharmaceutical compositions comprising the oligomeric compound ISIS 721744 encompass any pharmaceutically acceptable salt of the oligomeric compound ISIS 721744, esters of ISIS 721744, or salts of such esters. In certain embodiments, pharmaceutical compositions comprising ISIS 721744 are capable of providing (directly or indirectly) the biologically active metabolite or residue thereof upon administration to a human subject. Accordingly, for example, the disclosure is also drawn to pharmaceutically acceptable salts of the oligomeric compound ISIS 721744, prodrugs of ISIS 721744, pharmaceutically acceptable salts of such prodrugs, and other bioequivalents. Suitable pharmaceutically acceptable salts include, but are not limited to, sodium and potassium salts.

In certain embodiments, pharmaceutical compositions comprise one or more lipid moieties and the oligomeric compound ISIS 721744. In certain embodiments, lipid moieties are used to increase distribution of ISIS 721744 to a particular cell or tissue. In certain such methods, ISIS 721744 is introduced into preformed liposomes or lipoplexes made of mixtures of cationic lipids and neutral lipids. In certain methods, DNA complexes with mono- or poly-cationic lipids are formed without the presence of a neutral lipid.

In certain embodiments, pharmaceutical compositions disclosed herein comprise a delivery system. Examples of delivery systems include, but are not limited to, liposomes and emulsions. Certain delivery systems are useful for preparing pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver oligomeric compound ISIS 721744 to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV)). In certain of such embodiments, a pharmaceutical composition comprises a carrier and is formulated in aqueous solution, such as water, or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, the oligomeric compound ISIS 721744 acts as an acid. Although ISIS 721744 may be drawn or described in protonated (free acid) form, or ionized and in association with a cation (salt) form, aqueous solutions of ISIS 721744 exist in equilibrium among such forms. For example, a phosphate linkage of ISIS 721744 in aqueous solution exists in equilibrium among free acid, anion, and salt forms. Unless otherwise indicated, the term, “ISIS 721744,” is intended to include all such forms. Moreover, ISIS 721744 has several such linkages, each of which is in equilibrium. Thus, ISIS 721744 exists in solution in an ensemble of forms at multiple positions all at equilibrium. The term “ISIS 721744” is intended to include all such forms. Drawn structures necessarily depict a single form. Nevertheless, unless otherwise indicated, such drawings are likewise intended to include corresponding forms. Herein, a structure depicting the free acid of ISIS 721744 followed by the term “or a salt thereof” expressly includes all such forms that may be fully or partially protonated/de-protonated/in association with a cation. In certain instances, one or more specific cation is identified.

In certain embodiments, ISIS 721744 is in aqueous solution with sodium. In certain embodiments, ISIS 721744 is in aqueous solution with potassium. In certain embodiments, ISIS 721744 is in PBS. In certain embodiments, ISIS 721744 is in water. In certain such embodiments, the pH of the solution is adjusted with NaOH and/or HCl to achieve a desired pH.

Herein, certain specific doses are described. For clarity, a dose of ISIS 721744 in milligrams indicates the mass of the free acid form of ISIS 721744. As described above, in aqueous solution, the free acid is in equilibrium with anionic and salt forms. However, for the purpose of calculating dose, it is assumed that ISIS 721744 exists as a solvent-free, sodium-acetate free, anhydrous, free acid. For example, where ISIS 721744 is in solution comprising sodium (e.g., saline), ISIS 721744 may be partially or fully de-protonated and in association with Na+ ions. However, the mass of the protons is nevertheless counted toward the weight of the dose, and the mass of the Na+ ions are not counted toward the weight of the dose. Thus, for example, a dose of 80 mg of ISIS 721744 equals the number of fully protonated molecules that weighs 80 mg. This would be equivalent to 84 mg of solvent-free, sodium-acetate free, anhydrous sodiated ISIS 721744. Similarly, a dose of 100 mg of ISIS 721744 would be equivalent to 105 mg of solvent-free, sodium-acetate free, anhydrous sodiated ISIS 721744.

### IV. Certain Dosage Amounts

In certain embodiments, described herein are methods of administering to a subject a therapeutically effective amount of the oligomeric compound ISIS 721744. In certain embodiments, the therapeutically effective amount is about 20 mg. In certain embodiments, the therapeutically effective amount is about 40 mg. In certain embodiments, the therapeutically effective amount is about 60 mg. In certain embodiments, the therapeutically effective amount is about 80 mg. In certain embodiments, the therapeutically effective amount is about 100 mg. In certain embodiments, the therapeutically effective amount is 20 mg. In certain embodiments, the therapeutically effective amount is 40 mg. In certain embodiments, the therapeutically effective amount is 60 mg. In certain embodiments, the therapeutically effective amount is 80 mg. In certain embodiments, the therapeutically effective amount is 100 mg.

In certain embodiments, the therapeutically effective amount is any of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, and 300 mg.

In certain embodiments, the therapeutically effective amount is any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, and about 300 mg.

In certain embodiments, the therapeutically effective amount is any of 75.0 mg, 75.1 mg, 75.2 mg, 75.3 mg, 75.4 mg, 75.5 mg, 75.6 mg, 75.7 mg, 75.8 mg, 75.9 mg, 76.0 mg, 76.1 mg, 76.2 mg, 76.3 mg. 76.4 mg, 76.5 mg, 76.6 mg, 76.7 mg, 76.8 mg, 76.9 mg, 77.0 mg, 77.1 mg, 77.2 mg, 77.3 mg, 77.4 mg, 77.5 mg, 77.6 mg, 77.7 mg, 77.8 mg, 77.9 mg, 78.0 mg, 78.1 mg, 78.2 mg, 78.3 mg. 78.4 mg, 78.5 mg, 78.6 mg, 78.7 mg, 78.8 mg, 78.9 mg, 79.0 mg, 79.1 mg, 79.2 mg, 79.3 mg, 79.4 mg, 79.5 mg, 79.6 mg, 79.7 mg, 79.8 mg, 79.9 mg, 80.0 mg, 80.1 mg, 80.2 mg, 80.3 mg. 80.4 mg, 80.5 mg, 80.6 mg, 80.7 mg, 80.8 mg, 80.9 mg, 81.0 mg, 81.1 mg, 81.2 mg, 81.3 mg, 81.4 mg, 81.5 mg, 81.6 mg, 81.7 mg, 81.8 mg, 81.9 mg, 82.0 mg, 82.1 mg, 82.2 mg, 82.3 mg. 82.4 mg, 82.5 mg, 82.6 mg, 82.7 mg, 82.8 mg, 82.9 mg, 83.0 mg, 83.1 mg, 83.2 mg, 83.3 mg, 83.4 mg, 83.5 mg, 83.6 mg, 83.7 mg, 83.8 mg, 83.9 mg, 84.0 mg, 84.1 mg, 84.2 mg, 84.3 mg. 84.4 mg, 84.5 mg, 84.6 mg, 84.7 mg, 84.8 mg, 84.9 mg, and 85.0 mg.

In certain embodiments, the therapeutically effective amount is any of about 75.0 mg, about 75.1 mg, about 75.2 mg, about 75.3 mg, about 75.4 mg, about 75.5 mg, about 75.6 mg, about 75.7 mg, about 75.8 mg, about 75.9 mg, about 76.0 mg, about 76.1 mg, about 76.2 mg, about 76.3 mg, about 76.4 mg, about 76.5 mg, about 76.6 mg, about 76.6 about 76.7 mg, about 76.8 mg, about 76.9 mg, about 77.0 mg, about 77.1 mg, about 77.2 mg, about 77.3 mg, about 77.4 mg, about 77.5 mg, about 77.6 mg, about 77.7 mg, about 77.8 mg, about 77.9 mg, about 78.0 mg, about 78.1 mg, about 78.2 mg, about 78.3 mg, about 78.4 mg, about 78.5 mg, about 78.6 mg, about 78.7 mg, about 78.8 mg, about 78.9 mg, about 79.0 mg, about 79.1 mg, about 79.2 mg, about 79.3 mg, about 79.4 mg, about 79.5 mg, about 79.6 mg, about 79.7 mg, about 79.8 mg, about 79.9 mg, about 80.0 mg, about 80.1 mg, about 80.2 mg, about 80.3 mg, about 80.4 mg, about 80.5 mg, about 80.6 mg, about 80.7 mg, about 80.8 mg, about 80.9 mg, about 81.0 mg, about 81.1 mg, about 81.2 mg, about 81.3 mg, about 81.4 mg, about 81.5 mg, about 81.6 mg, about 81.7 mg, about 81.8 mg, about 81.9 mg, about 82.0 mg, about 82.1 mg, about 82.2 mg, about 82.3 mg, about 82.4 mg, about 82.5 mg, about 82.6 mg, about 82.7 mg, about 82.8 mg, about 82.9 mg, about 83.0 mg, about 83.1 mg, about 83.2 mg, about 83.3 mg, about 83.4 mg, about 83.5 mg, about 83.6 mg, about 83.7 mg, about 83.8 mg, about 83.9 mg, about 84.0 mg, about 84.1 mg, about 84.2 mg, about 84.3 mg, about 84.4 mg, about 84.5 mg, about 84.6 mg, about 84.7 mg, about 84.8 mg, about 84.9 mg, and about 85.0 mg.

In certain embodiments, the therapeutically effective amount is any of 10 mg to 140 mg, 10 mg to 130 mg, 10 mg to 120 mg, 10 mg to 110 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 140 mg, 20 mg to 130 mg, 20 mg to 120 mg, 20 mg to 110 mg, 20 mg to 100 mg, 20 mg to 90 mg, 20 mg to 80 mg, 20 mg to 70 mg, 20 mg to 60 mg, 20 mg to 50 mg, 20 mg to 40 mg, 20 mg to 30 mg, 30 mg to 140 mg, 30 mg to 130 mg, 30 mg to 120 mg, 30 mg to 110 mg, 30 mg to 100 mg, 30 mg to 90 mg, 30 mg to 80 mg, 30 mg to 70 mg, 30 mg to 60 mg, 30 mg to 50 mg, 30 mg to 40 mg, 40 mg to 140 mg, 40 mg to 130 mg, 40 mg to 120 mg, 40 mg to 110 mg, 40 mg to 100 mg, 40 mg to 90 mg, 40 mg to 80 mg, 40 mg to 70 mg, 40 mg to 60 mg, 40 mg to 50 mg, 50 mg to 140 mg, 50 mg to 130 mg, 50 mg to 120 mg, 50 mg to 110 mg, 50 mg to 100 mg, 50 mg to 90 mg, 50 mg to 80 mg, 50 mg to 70 mg, 50 mg to 60 mg, 60 mg to 140 mg, 60 mg to 130 mg, 60 mg to 120 mg, 60 mg to 110 mg, 60 mg to 100 mg, 60 mg to 90 mg, 60 mg to 80 mg, 60 mg to 70 mg, 70 mg to 140 mg, 70 mg to 130 mg, 70 mg to 120 mg, 70 mg to 110 mg, 70 mg to 100 mg, 70 mg to 90 mg, 70 mg to 80 mg, 80 mg to 140 mg, 80 mg to 130 mg, 80 mg to 120 mg, 80 mg to 110 mg, 80 mg to 100 mg, 80 mg to 90 mg, 90 mg to 140 mg, 90 mg to 130 mg, 90 mg to 120 mg, 90 mg to 110 mg, 90 mg to 100 mg, 100 mg to 140 mg, 100 mg to 130 mg, 100 mg to 120 mg, 100 mg to 110 mg, 110 mg to 140 mg, 110 mg to 130 mg, 110 mg to 120 mg, 120 mg to 140 mg, 120 mg to 130 mg, 130 mg to 140 mg, 65 mg to 95 mg, 65 mg to 90 mg, 65 mg to 85 mg 65 mg to 80 mg, 65 mg to 75 mg, 65 mg to 70 mg, 70 mg to 95 mg, 70 mg to 85 mg, 70 mg to 75 mg, 75 mg to 100 mg, 75 mg to 95 mg, 75 mg to 90 mg, 75 mg to 85 mg, 75 mg to 80 mg, 80 mg to 95 mg, 80 mg to 85 mg, 85 mg to 100 mg, 85 mg to 90 mg, 90 mg to 95 mg, 95 mg to 100 mg, 80 mg to 89 mg, 80 mg to 88 mg, 80 mg to 87 mg, 80 mg to 86 mg, 80 mg to 84 mg, 80 mg to 83 mg, 80 mg to 82 mg, 80 mg to 81 mg, 81 mg to 90 mg, 82 mg to 89 mg, 82 mg to 88 mg, 82 mg to 87 mg, 82 mg to 86 mg, 82 mg to 85 mg, 82 mg to 84 mg, 82 mg to 83 mg, 83 mg to 90 mg, 83 mg to 89 mg, 83 mg to 88 mg, 83 mg to 87 mg, 83 mg to 86 mg, 83 mg to 85 mg, 83 mg to 84 mg, 84 mg to 90 mg, 84 mg to 89 mg, 84 mg to 88 mg, 84 mg to 87 mg, 84 mg to 86 mg, 84 mg to 85 mg, 85 mg to 89 mg, 85 mg to 88 mg, 85 mg to 87 mg, 85 mg to 86 mg, 86 mg to 90 mg, 86 mg to 89 mg, 86 mg to 88 mg, 86 mg to 87 mg, 87 mg to 90 mg, 87 mg to 89 mg, 87 mg to 88 mg, 88 mg to 90 mg, 88 mg to 89 mg, and 89 mg to 90 mg.

In certain embodiments, the therapeutically effective amount is any of less than 300 mg, less than 295 mg, less than 290 mg, less than 285 mg, less than 280 mg, less than 275 mg, less than 270 mg, less than 265 mg, less than 260 mg, less than 255 mg, less than 250 mg, less than 245 mg, less than 240 mg, less than 235 mg, less than 230 mg, less than 225 mg, less than 220 mg, less than 215 mg, less than 210 mg, less than 205 mg, less than 200 mg, less than 195 mg, less than 190 mg, less than 185 mg, less than 180 mg, less than 175 mg, less than 170 mg, less than 165 mg, less than 160 mg, less than 150 mg, less than 145 mg, less than 140 mg, less than 135 mg, less than 130 mg, less than 125 mg, less than 120 mg, less than 115 mg, less than 110 mg, less than 105 mg, less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, less than 40 mg, less than 35 mg, less than 30 mg, less than 25 mg, and less than 20 mg.

In certain embodiments, the therapeutically effective amount is any of less than about 300 mg, less than about 295 mg, less than about 290 mg, less than about 285 mg, less than about 280 mg, less than about 275 mg, less than about 270 mg, less than about 265 mg, less than about 260 mg, less than about 255 mg, less than about 250 mg, less than about 245 mg, less than about 240 mg, less than about 235 mg, less than about 230 mg, less than about 225 mg, less than about 220 mg, less than about 215 mg, less than about 210 mg, less than about 205 mg, less than about 200 mg, less than about 195 mg, less than about 190 mg, less than about 185 mg, less than about 180 mg, less than about 175 mg, less than about 170 mg, less than about 165 mg, less than about 160 mg, less than about 150 mg, less than about 145 mg, less than about 140 mg, less than about 135 mg, less than about 130 mg, less than about 125 mg, less than about 120 mg, less than about 115 mg, less than about 110 mg, less than about 105 mg, less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, less than about 40 mg, less than about 35 mg, less than about 30 mg, less than about 25 mg, and less than about 20 mg.

In certain embodiments, the therapeutically effective amount is any of at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least about 100 mg, at least 105 mg, at least 115 mg, at least 120 mg, at least 125 mg, at least 130 mg, at least 135 mg, at least 140 mg, at least 145 mg, and at least 150 mg. In certain embodiments, the therapeutically effective amount does not exceed about 200 mg, about 250 mg, about 300 mg, about 350 mg, or about 400 mg.

In certain embodiments, the therapeutically effective amount is any of at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 115 mg, at least about 120 mg, at least about 125 mg, at least about 130 mg, at least about 135 mg, at least about 140 mg, at least about 145 mg, and at least about 150 mg. In certain embodiments, the therapeutically effective amount does not exceed about 200 mg, about 250 mg, about 300 mg, about 350 mg, or about 400 mg.

### V. Certain Dosing Regimens

In certain embodiments, described herein are methods of administering to a subject a therapeutically effective amount of the oligomeric compound ISIS 721744 one or more times. In certain embodiments, methods comprise administering the therapeutically effective amount at least 1, 2, 3, 4, 5, 6, 7, 8, 9 or 10 times. In certain embodiments, methods comprise administering the therapeutically effective amount once about every 2 weeks. In certain embodiments, methods comprise administering the therapeutically effective amount once every about every 3 weeks. In certain embodiments, methods comprise administering the therapeutically effective amount once about every 4 weeks. In certain embodiments, methods comprise administering the therapeutically effective amount once about every 8 weeks. In certain embodiments, methods comprise administering the therapeutically effective amount once every 2 weeks. In certain embodiments, methods comprise administering the therapeutically effective amount once every 3 weeks. In certain embodiments, methods comprise administering the therapeutically effective amount once every 4 weeks. In certain embodiments, methods comprise administering the therapeutically effective amount once every 8 weeks.

In certain embodiments, methods comprise administering the therapeutically effective amount about every 1 day, about every 2 days, about every 3 days, about every 4 days, about every 5 days, about every 6 days, about every 7 days, about every 8 days, about every 9 days, about every 10 days.

In certain embodiments, methods comprise administering the therapeutically effective amount about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, or about every 20 weeks.

In certain embodiments, methods comprise administering the therapeutically effective amount for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months.

### Loading and Maintenance Doses

In certain embodiment, the therapeutically effective amount is administered as a loading dose and/or a maintenance dose. In certain embodiments, methods comprise administering a loading dose or doses and subsequently administering a maintenance dose or doses. In certain embodiments, methods comprise administering a loading dose once about every 3 weeks, and subsequently administering a maintenance dose once about every 4 weeks. In certain embodiments, methods comprise administering a loading dose once about every 4 weeks, and subsequently administering a maintenance dose once about every 8 weeks.

In certain embodiments, methods comprise administering at least 2 loading doses, at least 3 loading doses, at least 4 loading doses, at least 5 loading doses, or at least 6 loading doses. In certain embodiments, methods comprise administering 2, 3, 4, 5, 6, 7, 8, 9, 10, 11, 12, 13, 14, 15, or 16 loading doses. In certain embodiments, methods comprise administering a loading dose or doses once about every 1 week, once about every 2 weeks, once about every 3 weeks, once about every 4 weeks, once about every 5 weeks, once about every 6 weeks, once about every 7 weeks, once about every 8 weeks, once about every 9 weeks, once about every 10 weeks, once about every 11 weeks, or once about every 12 weeks. In certain embodiments, methods comprise administering an initial loading dose and administering a second loading dose about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks after administering the initial loading dose.

In certain embodiments, methods comprise administering at least 2 maintenance doses, at least 3 maintenance doses, at least 4 maintenance doses, at least 5 maintenance doses, or at least 6 maintenance doses. In certain embodiments, methods comprise administering 2, 3, 4, 5, 6, 7, 8, 9, 10, 11, 12, 13, 14, 15, or 16 maintenance doses. In some instances, methods comprise administering a maintenance dose or doses once about every 4 weeks, once about every 5 weeks, once about every 6 weeks, once about every 7 weeks, once about every 8 weeks, once about every 9 weeks, once about every 10 weeks, once about every 11 weeks, once about every 12 weeks, once about every 13 weeks, once about every 14 weeks, once about every 15 weeks, once about every 16 weeks, once about every 17 weeks, once about every 18 weeks, once about every 19 weeks, or once about every 20 weeks. In certain embodiments, methods comprise administering a first maintenance dose and administering a second maintenance dose about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks after administering the first maintenance dose.

In certain embodiments, methods comprise administering a first maintenance dose or doses about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks after administering the last loading dose.

### VI. Potency and Efficacy

In certain embodiments, described herein are methods of reducing PKK RNA, PKK protein, and/or PKK activity in a cell or biological fluid of a human subject, wherein the methods comprise administering a therapeutically effective amount of ISIS 721744 to the subject. In certain embodiments, methods reduce PKK RNA, PKK protein, and/or PKK activity in the blood of the human subject. In certain embodiments, methods reduce PKK RNA, PKK protein, and/or PKK activity in the liver of the human subject. One may determine whether or not methods reduce PKK RNA, PKK protein, and/or PKK activity, e.g., by detecting/quantifying a first amount of PKK RNA, PKK protein, and/or PKK activity in a first biological sample obtained before administering and detecting/quantifying a second amount of PKK RNA, PKK protein, and/or PKK activity in a second biological sample obtained after administering, and detecting or quantifying a reduction in PKK RNA, PKK protein, and/or PKK activity by comparing the first amount to the second amount.

In certain embodiments, methods comprise reducing PKK RNA, PKK protein, PKK activity, or a combination thereof, by 1-100%, or a range defined by any two of these values. In certain embodiments, methods comprise reducing PKK RNA and/or PKK protein by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In certain embodiments, methods comprise reducing PKK RNA, PKK protein, PKK activity, or a combination thereof, in a cell or a subject, by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In certain embodiments, methods comprise reducing PKK RNA, PKK protein, PKK activity, or a combination thereof, in a cell or a subject, by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to 100%.

In certain embodiments, methods comprise administering ISIS 721744 to a subject and detecting or quantifying PKK RNA, PKK protein, PKK activity, or a combination thereof, in a cell or a biological fluid of the subject. In certain embodiments, methods comprise detecting/quantifying PKK RNA, PKK protein, PKK activity, or a combination thereof, in a first biological sample obtained before administering; detecting/quantifying PKK RNA, PKK protein, PKK activity, or a combination thereof, in a second biological sample obtained after administering; and detecting or quantifying a reduction in PKK RNA, PKK protein, PKK activity, or a combination thereof, by comparing the first amount to the second amount. In certain embodiments, the second biological sample is obtained less than about 24 hours after administering. In certain embodiments, the second biological sample is obtained less than about 1 week after administering. In certain embodiments, the second biological sample is obtained about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, or about 18 weeks after administering. In certain embodiments, methods comprise increasing or decreasing the dose after comparing the first amount to the second amount. In certain embodiments, methods comprise administering more frequently or less frequently after comparing the first amount to the second amount.

### Assessing Efficacy of ISIS 721744

### A. Hereditary Angioedema

In certain embodiments, methods described herein are sufficiently effective to ameliorate or prevent at least one symptom of hereditary angioedema in a human subject. In certain embodiments, the least one symptom of hereditary angioedema is selected from swelling, nausea, vomiting, itching, headache, fatigue, abdominal pain, shortness of breath, rhinitis, anaphylaxis, and bronchoconstriction, and a combination thereof.

In certain embodiments, methods described herein are sufficiently effective to reduce the occurrence of angioedema attacks in a subject with HAE. In certain embodiments, methods described herein reduce the occurrence of angioedema attacks by at least about 10%, at least about 20%, at least about 30%, at least about 40%, at least about 50%, at least about 60%, at least about 70%, at least about 80% or at least about 90%, as measured over at least about 1 month, at least about 2 months, at least about 3 months, at least about 4 months, about 5 months, at least about 6 months, at least about 7 months, at least about 8 months, at least about 9 months, at least about 10 months, at least about 11 months, at least about 12 months. In certain embodiments, the angioedema attacks comprise breakthrough attacks.

In certain embodiments, methods described herein are sufficiently effective to reduce the severity of an angioedema attack in a subject with HAE. In certain embodiments, methods described herein are sufficiently effective to reduce swelling of a body part of a subject having an angioedema attack by 10, 15, 20, 25, 30, 35, 40, 45, 50, 55, 60, 65, 70, 75, 80, 85, 90, 95 or 99%, or a range defined by any two of these values. A reduction in swelling may be measured, e.g., with a measuring tape around a body part (e.g., abdomen, arm, wrist, ankle, neck) before and after administering oligomeric compound ISIS 721744. In certain embodiments, the angioedema attack is a breakthrough attack.

In certain embodiments, methods described herein are sufficiently effective to reduce the severity or occurrence of angioedema attacks in a subject with HAE who is refractory to an anti-edema agent. In certain embodiments, the subject who is refractory to an anti-edema agent experiences less than about 5%, less than about 10%, less than about 20%, less than about 30%, less than about 40%, or less than about 50% reduction in the frequency of angioedema attacks as measured over at least 1, at least 2, at least 3, at least 4 or at least 6 months after treatment with at least one anti-edema agent relative to before treatment with the at least one anti-edema agent. In certain embodiments, the subject who is refractory to at least one anti-edema agent experiences no reduction in the frequency of angioedema attacks as measured over at least 1, at least 2, at least 3, at least 4 or at least 6 months after treatment with at least one anti-edema agent relative to before treatment with the at least one anti-edema agent. In certain embodiments, the subject who is refractory to an anti-edema agent experiences less than about 5%, less than about 10%, less than about 20%, less than about 30%, less than about 40%, or less than about 50% reduction in swelling as measured about 1 hour, about 2 hours, about 4 hours, about 8 hours, about 12 hours, about 24, or about 48 hours after treatment with the at least one anti-edema agent relative to before treatment with the at least one anti-edema agent. In certain embodiments, the subject who is refractory to an anti-edema agent continues to have at least one symptom of an angioedema attack as observed about 1 hour, about 2 hours, about 4 hours, about 8 hours, about 12 hours, about 24, or about 48 hours after treatment with the at least one anti-edema agent. In certain embodiments, the subject who is refractory continues to have one or more symptoms of hereditary angioedema when treated prophylactically with one or more anti-edema agents. In certain embodiments, the subject who is refractory continues to have angioedema attacks when treated prophylactically with one or more anti-edema agents. In certain embodiments, the angioedema attacks comprise breakthrough attacks.

### B. Macular Edema

In certain embodiments, methods described herein are sufficiently effective to ameliorate at least one symptom of macular edema in a human subject. In certain embodiments, the least one symptom of macular edema is selected from blurry vision, wavy vision, distorted vision, loss of vision, and a combination thereof.

In certain embodiments, methods described herein are sufficiently effective to ameliorate vision of a subject having macular edema, as assessed by central subfield thickness (CST), also known as (central) foveal thickness. The CST is the average thickness of the macula in the central-most area of the macula having a 1 mm diameter, and can be measured with a Zeiss Cirrus instrument or a Heidelberg Spectralis instrument. In certain embodiments, subjects with macular edema have a CST of at least about 300 μm, at least about 310 μm, at least about 320 μm, at least about 330 μm, at least about 340 μm, or at least about 350 μm before administering. In certain embodiments, methods reduce the subject's CST by at least about 10 μm, at least about 20 μm, at least about 30 μm, at least about 40 μm, at least about 50 μm, or at least about 60 μm.

In certain embodiments, methods described herein are sufficiently effective to ameliorate the visual acuity or best-corrected visual acuity (BCVA) of a subject having macular edema, as assessed by a Snellen Chart, Sloan Chart or Early Treatment Diabetic Retinopathy Study (ETDRS) chart. These charts provide lines of letters, the letters becoming progressively smaller with each subsequent line. The chart is held at a distance from a subject, and the subject is asked to read the letters. The subject receives a score based on their ability to read lines of letters. In certain embodiments, subjects with macular edema have a visual acuity score of less than 20/25, less than 20/32, less than 20/40, less than 20/50, or less than 20/63, as assessed by the Snellen chart, or an equivalent score on the Sloan Chart or ETDRS chart. In certain embodiments, methods increase visual acuity on any one of these charts by at least 1, at least 2, at least 3, at least 4, at least 5, at least 6, at least 7, at least 8, at least 9, or at least 10 letters.

In certain embodiments, methods described herein are sufficiently effective to ameliorate vision of a subject having macular edema, improving the subject's rating on the diabetic retinopathy severity scale (DRSS) as assessed by the color fundus photography. The DRSS is described in greater detail by Staurenghi et al., Br J Ophthalmol. 2018; 102: 954-958. In certain embodiments, methods provide at least a 2-step improvement of the subject's DRSS score.

### VI. Certain Combination Therapies

In certain embodiments, methods comprise co-administering ISIS 721744 with at least one other pharmaceutical agent. In certain embodiments, the at least one other pharmaceutical agent ameliorates edema. In certain embodiments, edema is hereditary angioedema. In certain embodiments, edema is macular edema. In certain embodiments, ISIS 721744 is co-administered with the at least one other pharmaceutical agent to produce a combinational effect. In certain embodiments, ISIS 721744 is co-administered with the at least one other pharmaceutical agent to produce a synergistic effect.

In certain embodiments, ISIS 721744 and the at least one other pharmaceutical agent are administered at the same time. In certain embodiments, ISIS 721744 and the at least one other pharmaceutical agent are administered at different times. In certain embodiments, ISIS 721744 and the at least one other pharmaceutical agent are prepared together in a single formulation. In certain embodiments, ISIS 721744 and the at least one other pharmaceutical agent are administered are prepared separately.

In certain embodiments, ISIS 721744 is co-administered with an anti-edema agent to a subject with HAE. Non-limiting examples of anti-edema agents include C1-INH protein, an angiotensin-converting enzyme inhibitor, an angiotensin II type-1 receptor blocker (ARB), an antihistamine, a corticosteroid, an antifibrinolytic (e.g., tranexamic acid), a bradykinin inhibitor, bradykinin type-2 receptor blocker (e.g., icatibant), a kallikrein inhibitor (e.g., lanadelumab, ecallantide, BCX7353), and an attenuated androgen (e.g., danazol, stanozolol, oxandrolone). In certain embodiments, the anti-edema agent is plasma-derived C1-INH concentrate or recombinant C1-INH concentrate. In certain embodiments, the subject is female and the anti-edema agent comprises gonadotropin releasing hormone, or an analog thereof, that is capable of depleting estrogen or inhibiting estrogen activity in the subject.

In certain embodiments, ISIS 721744 is co-administered with at least one of a corticosteroid, nonsteroidal anti-inflammatory drug (NSAID), and vascular endothelial growth factor (VEGF) inhibitor, (e.g., anti-VEGF antibody), to a subject with macular edema.

In certain embodiments, ISIS 721744 is co-administered with an anti-thrombotic agent to a subject in need thereof. Non-limiting examples of anti-thrombotic agents include nonsteroidal anti-NSAIDs, cyclooxygenase inhibitors (e.g., aspirin), adenosine diphosphate (ADP) receptor inhibitors (e.g., clopidogrel and ticlopidine), phosphodiesterase inhibitors (e.g., cilostazol), glycoprotein IIB/IIIA inhibitors (e.g., abciximab, eptifibatide, tirofiban, and defibrotide), adenosine reuptake inhibitors, (e.g., dipyridamole), coumarins (e.g., warfarin), heparin and derivatives thereof (e.g., enoxaparin), direct thrombin inhibitors (e.g., lepirudin, bivalirudin), apixaban, and small molecular compounds that interfere directly with the enzymatic action of particular coagulation factors (e.g. rivaroxaban, which interferes with Factor Xa).

In certain embodiments, ISIS 721744 is co-administered with an anti-inflammatory agent to a subject in need thereof. Non-limiting examples of anti-inflammatory agents include methotrexate, abatacept, infliximab, cyclophosphamide, azathioprine, corticosteroids, cyclosporin A, aminosalicylates, sulfasalazine, hydroxychloroquine, leflunomide, etanercept, efalizumab, 6-mercapto-purine (6-MP), and tumor necrosis factor-alpha (TNFalpha), and other cytokine blockers or antagonists. In certain embodiments, the anti-inflammatory agent comprises a non-steroidal anti-inflammatory drug (NSAID). NSAIDS include, but are not limited to, acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, meloxicam and tramadol.

In certain embodiments, methods comprise administering ISIS 721744 to a subject in need thereof and performing plasmapheresis on the subject before, during, and/or after administering.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Phase 1 Human Clinical Trial with ISIS 721744

ISIS 721744 was provided in a sterile saline solution at a concentration of 100 mg/mL. Varying doses of ISIS 721744 were tested in a randomized, double-blind, placebo-controlled study to evaluate the safety, tolerability, pharmacokinetics and pharmacodynamics of ISIS 721744 in healthy volunteers. Subjects received four monthly subcutaneous injections of placebo, or ISIS 721744, at doses of 20, 40, 60 or 80 mg for each injection.

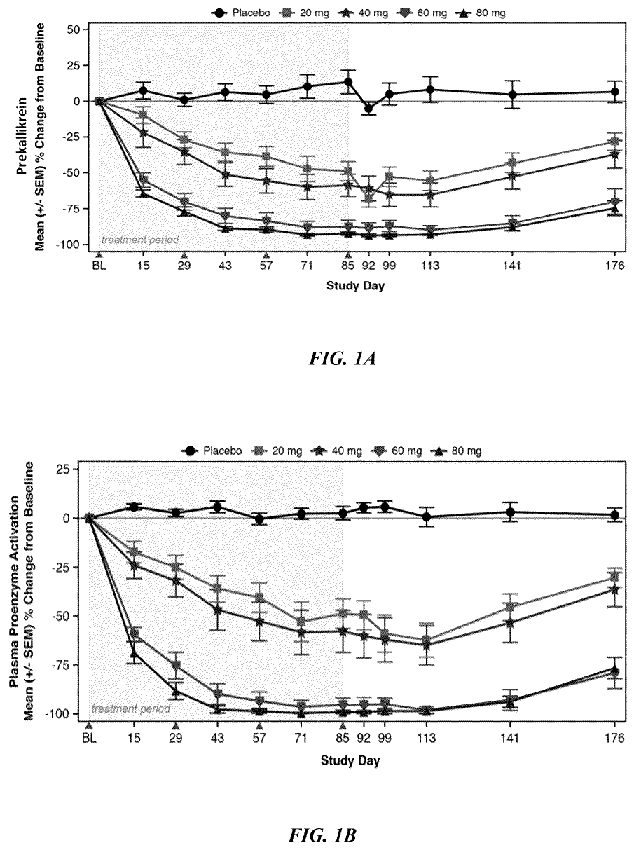

ISIS 721744 produced a dose-dependent mean reduction in plasma PKK protein (FIG. 1A) concentration and proenzyme activation (FIG. 1B). Proenzyme activation was measured as described by Ferrone et al., “IONIS-PKK_{Rx} a Novel Antisense Inhibitor of Prekallikrein and Bradykinin Production,” 2019, Nucleic Acid Ther., (29):82-91. Mean reductions in plasma PKK levels across dose groups ranged from 52.9% to 93.6% below baseline. In the 80 mg group, plasma PKK protein levels showed a mean (±SD) reduction from baseline of 93.6% (±2.3) two weeks after the last dose. Pharmacology was sustained for three months after the last dose in this dose group with a mean 74.7% (±12.7) reduction from baseline in PKK levels. Similarly, plasma proenzyme activation showed a mean (±SD) reduction from baseline of 98.6% (±2.0) at two weeks and 76.6% (±13.4) at three months after the last dose in the 80 mg dose group.

No serious adverse event occurred, and no subject discontinued dosing due to a treatment-emergent adverse event (TEAE). There were no dose or time-dependent changes in laboratory tests for coagulation, aPTT and prothrombin.

### Example 2: Efficacy Proof-of-Concept Study in Type I HAE Patient

A patient with type I HAE was selected to assess clinical efficacy and tolerability of ISIS 721744. This patient had a history of life-threatening angioedema attacks on an average of twice a month. This patient also had a reported intolerance to pharmaceutical agents that have been approved for the treatment of HAE, including tranexamic acid, attenuated androgens, and C1-inhibitor concentrate.

Eight weeks after prior prophylactic treatment was discontinued, the patient was administered ISIS 721744 subcutaneously at a dose of 80 mg. This dose was administered monthly for 8 months. Plasma PKK activity was measured at least monthly. Briefly, a plasma sample from the patient was added to PKK-deficient plasma and the resulting sample was compared to samples of known PKK concentration in an activated partial thromboplastin time (aPTT) test, a blood test that characterizes coagulation of the blood. Table 1 shows plasma PKK activity (relative to baseline for prior prophylactic treatment) was reduced over the course of treatment with ISIS 721744. Plasma PKK activity was reduced ˜4-fold after 6 months of treatment with ISIS 721744. No angioedema attacks were reported during the 8 months of treatment with ISIS 721744. No other prophylactic HAE therapies have been reported to eliminate all breakthrough attacks for such a duration. Treatment was well tolerated, and no adverse events at the injection site were reported.

TABLE 1
Plasma PKK activity in type I HAE
patient administered ISIS 721744

| Study Day | 1 | 29 | 57 | 90 | 118 | 148 | 182 | 208 |
|---|---|---|---|---|---|---|---|---|
| % PKK Activity | 24 | 7 | 5 | 20 | 10 | 19 | 6 | 5 |

### Example 3: Efficacy Proof-of-Concept Study in Type III HAE Patient

A patient with type III HAE was selected to assess clinical efficacy and tolerability of ISIS 721744. This patient had normal level and function of C1-INH and no typical mutations for nl-C1-INH-HAE. This patient had a history of life-threatening angioedema attacks (6-10 attacks per month), despite all available therapeutic options, including weekly plasma exchange with albumin/NaCl solution. This patient was either refractory to, or intolerant of, histamine inhibitor, antifibrinolytic (tranexamic acid), attenuated androgen (danazol), C1-inhibitor concentrate, and bradykinin B2 receptor antagonist (icatibant).

Seven weeks after prior prophylactic treatment was discontinued, the patient was administered ISIS 721744 subcutaneously at a dose of 80 mg. This dose was administered again four weeks later. In the subsequent weeks, a series of severe angioedema attacks were reported following multiple hospitalizations due to H. influenza pneumonia with respiratory failure, a catheter associated bloodstream infection, and surgery. The dosing interval was increased to once every three weeks during the first hospitalization, which occurred seven weeks from the first ISIS 721744 dosing. Apart from the aforementioned hospitalizations, the frequency of attacks was reduced to one attack per month, as measured over the 9 months that ISIS 721744 was administered. Weekly plasma exchange was successfully discontinued 8 weeks after the last hospitalization. Table 2 shows plasma PKK activity (relative to baseline for prophylactic treatment) was reduced over the course of treatment with ISIS 721744. Plasma PKK activity was measured as described in Example 2. Administration of ISIS 721744 was well tolerated and no injection-site reactions were seen.

TABLE 2
Plasma PKK activity in type III HAE patient administered ISIS 721744

| Study Day | 1 | 30 | 51 | 90 | 111 | 132 | 154 | 175 | 196 | 217 | 237 |
|---|---|---|---|---|---|---|---|---|---|---|---|
| % PKK Activity | 60 | 20 | 14 | 15 | 19 | 8 | 16 | 15 | 32 | 33 | 31 |

### Example 4: Phase 2 Human Clinical Trial of ISIS 721744 for HAE

ISIS 721744 is evaluated in a Phase 2 multicenter, randomized, placebo-controlled study in patients with type I HAE, type II HAE, or type III HAE. Patients with type III HAE may or may not have a mutation in a gene selected from F12, PLG, and ANGPTL1. In an eight-week screening period, patients must experience a minimum of two angioedema attacks. Patients are randomized to receive subcutaneous treatment with 80 mg ISIS 721744 or placebo every four weeks for at least twelve weeks. Patients who are not attack free during these twelve weeks are switched to a dose of 100 mg. Patients who are attack free during these twelve weeks have the frequency of administration reduced to every eight weeks.

Pharmacodynamics and efficacy are assessed at multiple time points, including measuring cleaved high molecular weight kininogen (cHK) levels and/or percent change in PKK activity in plasma. PKK activity is determined as described in Example 2. The primary endpoint is the time-normalized number of angioedema attacks per month. Patient safety is monitored closely during the study. Safety and tolerability evaluations include e.g. physical examination, vital signs, adverse events, concomitant medications, and plasma laboratory tests.

### Example 5: Phase 2 Human Clinical Trial of ISIS 721744 for Macular Edema

Safety and efficacy of ISIS 721744 in patients with diabetic macular edema are evaluated in a randomized, double-masked, placebo-controlled Phase 2 study. Some patients may have previously received another treatment for macular edema, e.g., anti-VEGF antibody or other VEGF inhibitor treatment. Patients are randomized to receive subcutaneous treatment with 80 mg ISIS 721744 or placebo at Weeks 1, 3, 5, 9, and 13.

Pharmacodynamics and efficacy are assessed at multiple time points. The primary endpoint is the proportion of patients having a >60 μm reduction of central retinal subfield thickness (CST) at 17 and 25 weeks from first dose. The secondary endpoint is the proportion of patients having mean increase ≥5 best corrected visual acuity (BCVA) at 17 and 25 weeks from first dose. Additional exploratory endpoints include mean change in CST and BCVA score; ≥2-step improvement of diabetic retinopathy severity scale (DRSS); resolution of macular edema defined as CST to either <300 μm on the Cirrus or <320 μm on the Heidelberg Spectralis instrument; visual acuity letter score improvement of 10 letters (2 lines gain) or 15 letters (3 lines gains); and absolute and relative reduction in plasma PKK RNA concentration and/or plasma PKK protein concentration over time. Patient safety is monitored closely during the study. Safety and tolerability evaluations include e.g. physical examination, vital signs, adverse events, concomitant medications, and plasma laboratory tests.

1. A method of ameliorating edema in a human subject in need thereof, the method comprising administering to the human subject a therapeutically effective amount of an oligomeric compound according to the following chemical structure: 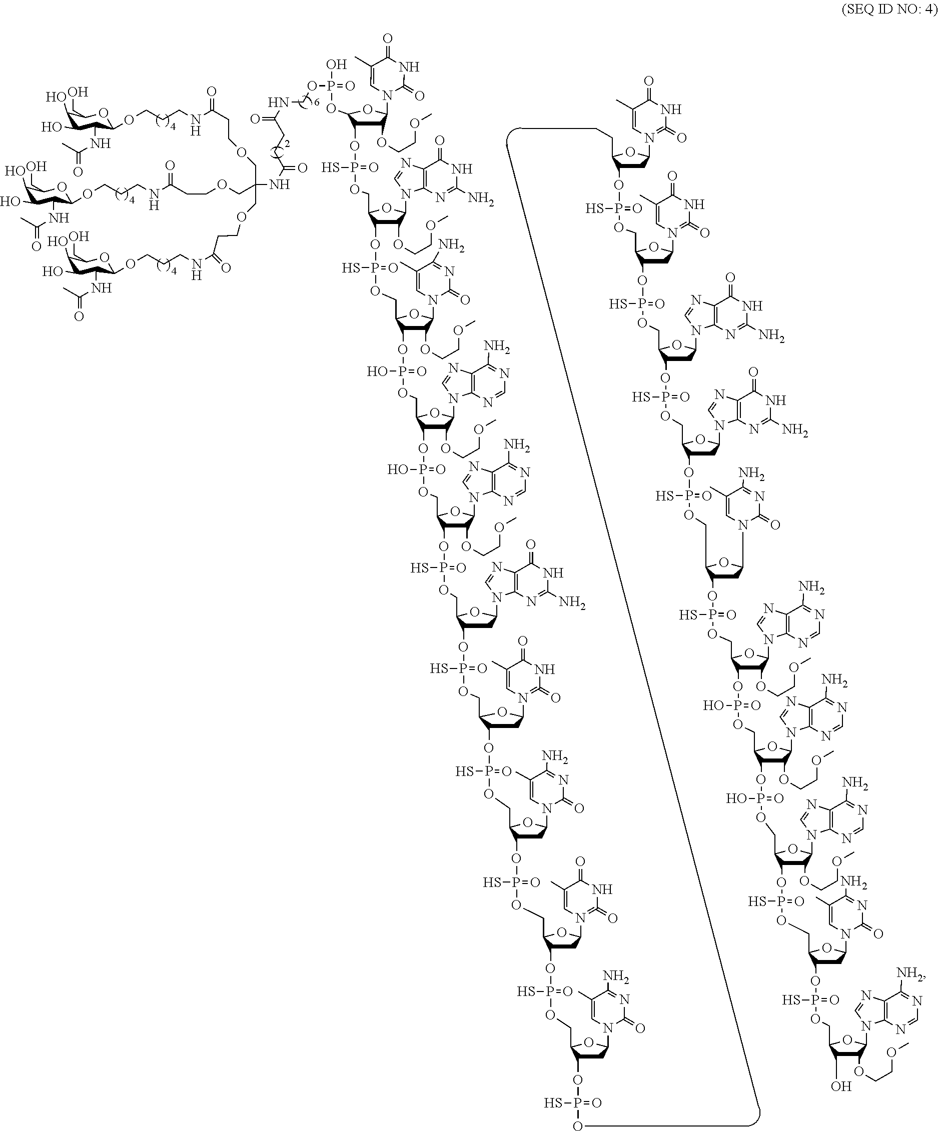
or a salt thereof.

2. The method of claim 1, wherein the salt is the sodium salt or the potassium salt.

3. A method of ameliorating edema in a human subject in need thereof, the method comprising administering to the human subject a therapeutically effective amount of an oligomeric compound according to the following chemical structure:

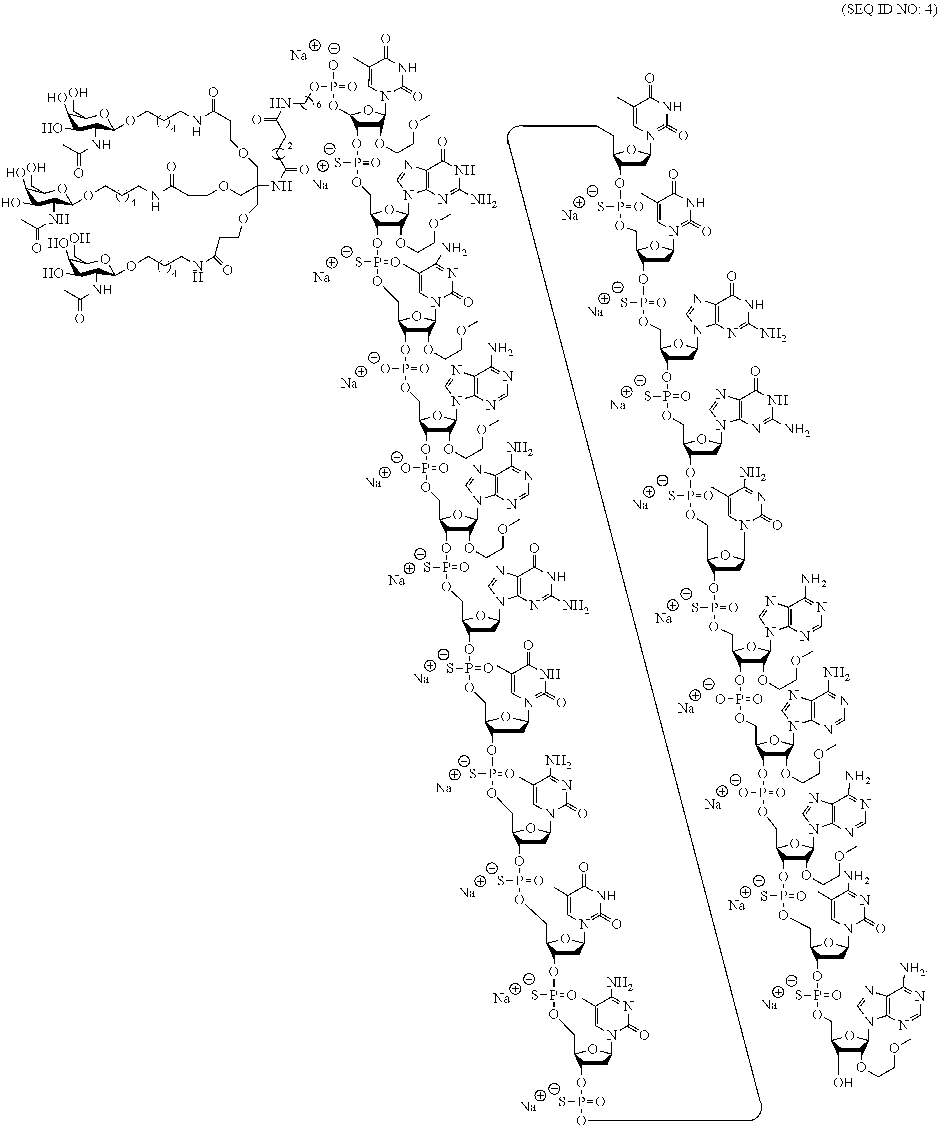

4. (canceled)

5. The method of claim 3, wherein edema is hereditary angioedema, and at least one symptom of hereditary angioedema is ameliorated.

6. The method of claim 5, wherein at least one symptom is selected from nausea, vomiting, itching, headache, fatigue, abdominal pain, shortness of breath, rhinitis, anaphylaxis, bronchoconstriction, and swelling, and a combination thereof.

7. The method of claim 3, wherein edema is macular edema, and at least one symptom of macular edema is ameliorated.

8. The method of claim 7, wherein at least one symptom is selected from blurry vision, wavy vision, distorted vision, and loss of vision, and a combination thereof.

9.-104. (canceled)


SEQUENCE LISTING (5 sequences)

SEQ ID NO: 1 - DNA, 37000 bp, Homo sapiens
  source 1..37000: mol_type = genomic DNA
agagcctctgcctagatttcacaggatgtgtggaaacacctggctgtccaggcagaagcctgctgcagggacccagccctcatggagaacctctattagggcagtgcagaggtgaactgtggggttggagcccacacacagagaccccactggagcactgcccagtggagctgtgaaaagagttcccccatcttccagatgccagaatggtagatccactgactggaaagttgcaggcactcagtgccggcccatgaaggcagccgtaggggctgtaccctgcaaagccacagggacagagctgcccaaggccttaacagcccacaccttgcatcagcatgccctgggtgtgagacaggggctccaaggagattattttggagctttaaaatttaatgacttccctgctaggttttgcatatgcttgggacctgtggcccctttattttggccaatttctcccatttggaatgggaacatttacccaattcctgtacccacattgtgttttggaagtaactaacttgttttttattttaaaggctcattgagggaagggacttgccttgtctcaaatgagactttggactcggacttttgggttactgtggaatgagttaagactttgggggactgttggaaagacatcattggttttgaaatctgaaaaggacatgagatttgggaaaggccaggggaagaatgatatggtttggctctgtgtcctcatccaaatctcgtcttgaattgtaatccccacatgggattacatgggaggggcctggtgagaggtgattggatcatggaggtggtttcccctgtgctgtgatctcatgatagtgagggatttaaaagtgacagtttcccctgcacatacacactctctctctcgtatcaccttgtgaagaaggtgtctgcttcccctctgccttccatcgtgattataagtttcctgaggcctccccagccatgggtaactgtgagtcagttaaacctcttttgtttataaattacccagtctcaggtagtatctttacagcagtgtgaaactggactaatacatctgggaaattctttttgtctccttcctttctgaaggacagctttgcctgatattatattcttggttgccaggttttgtactttcaatactttgaatatatcatcccactctctcttggcctacatgatttctgctgagaaatccaccaataatcttatgaagcttcccttgtatgtgaagaattgcttttctcttgcttctttgaaaattctctctttgtcatggtgaggatttcttttttttttttttttttttagatggagtctagctctgttgtccaggctggagtgcagtgtgcagtggcgcaatctcggctcactgcaagctccgtctcctgggttcacaccattttcctgtctcagcctctggagtagctgagactacaggtgcccaccaccacgcctggctaaattttttttgtatttttagtagagatggggtttcactgtgttagccaggatggtctcgatctcctgacctcatgatccacccatcttggcctcccaaagtgctgggattacaggcatgagccactgcacctggccgaggatctctttatatttaatctatttggagttcttttgggcttcataaatctggatgtttatttcatttatgtctccaaattttgagagttttctattcttatttttttaaataagtttctgcaactttctctttctctacttcttctggaactcttctaatgcatattaattttcttaacagtgtcctataagtcttgtcagctttctgcaatttttatcattccttttattactctgactgggtaatttcaaatgacctgtatctgagcatgctgattctttctcctgcttgatctagtctgctgctgaagctgtttgtgaattttttcaattcagtcattttgttctttagctccagaatttctgtttcattcttttttatggttcctatctatctttttgttgaacttctaattttgctcatgtattgttttcctgattttgtttactgtctatctatattgttttgtagcttattgagctttcttaaggcaattatttttagctcttgtcatgcagtaaagatctccatttctttagggtcacctcctgctgctttatttccttcctttggtggtttcttgtttccccgattatttgtgatcagtgtggccttgcactgaagtaggcacctatttcagtctttacatactagcttcagcagagaaagccattcactagtcagattgaccagagattgttggtgggctgtctgttggggtctatgcacaggatttctgctggagttctaaggtgggaagggctggattctgggttcattcactgtggttgtctgtattctgtgcacaaggactggcttgaagcatggatccttgggggctgacttggcactgaaatgagccttaagcctgcgtctgcaggggccagcctaacatggggatcacctggcacctgagttcatggggataggcctgttcctgagtttattcaggctgtcctgggaacaaggtccactggggtgagcccagcatctgggtccacatggcccagcatggagccaagatctctggaggctgacctggtgctggatctgcaggggatggcctggattctaggcccatgggtgccaacttggagcctggggttgctggggctaacgtggaggctagatagagtcttgggggccaggctagagctggagcaggcctgaagtctaggttttgtgtggccatcttggagcctgaagccccaggggctgacctggtctggggtgagcatggggctgaggccacagaggctggtctggcctgtggcaggcctgaatcctggtgctggggtttactggagtgggcttggtgcttgggatctgtggtgaagttaggttctatcttaactgtccctcctccatgcaagagggcatctctctccatactgtgctgcccaggcttgaaggtgagatgacaccggtaatgtgaaattgtccttcctatacacttctatgtgtcttttcttatttctgtgctgcaaccaggtggcataacctctcacctgattccttagctctagtgaagttattttcgtgcatggatacttgttcgaattgatgtttctgcaagggatgagcgctagaaactcctgttctgacaaactcctattcctattcttgctgacatcactccctgaaatagttaatatacttaacagctgaacacggataggatgttcatggaatatgttgacaggacaaaaagttgaaactgttggcagaaacccaaagtcaatattgaagccaagcaaaatattgcctgcagtgccacattagaacagcttgaagaccgttcatttttaagtgacaagagactcacctccaagaagcaattgtgttttcaggtagcaaatttttattattctgattgtttccaaataaactataatttttaagtataattttttactttatgagaaaattaatcatttatattctaatttcctgagtatgtagagagtatagataatgttcctttatgtagaaatatttaaatgtaagatgattttaaatcagaaagaatatttgattgatttaaaatttttaaatgggctttaatattttcagaggttttctttacttagggatttttggactgacattattgccattatttattaattttgtttttgcccaaatcaagaggtttcataattgtttactctctctctaccaattccctttccaacattactagccacagagttggccaatgaacaataaacacaacagtagtctggaggtctcaatttgtatcttgggaagcattataaattttccaactccctagacacaaatgtaccaaaaaaaaacccttgttttctataccagtaattgtgtgctttgtcttgcaattcagacatttacaagaaaatctaaatcaccttaaattaagatttatgttaaatgtggtctaaaaccagcagagttatgtattgttttcttttttagaatgattttattcaagcaagcaacttatttcatttccttgtttgctacagtttcctgtggtaagtgaattatctataaaacatggaattcaggctaagacaggagtagccaagcaagtggcaccacccctggagaaagctattgaacatacagcttcgggggtggagattgtccctgatgattcaggacacgtgtctatttaatgttccacaacaaggaccacttgtcaggtatattgctgtagacatatgttgcagaccagaggaaggagctcagaagtaggaatgtcttgggacttgtgttaacaaaaacttctgttcgcagatgacactctgcaaagcaaaacttgaaacaaaaaaaaattagtcctctatttttattatcaacagtaaaaaattaaactttatctgaaaattcaaaagagtgctaggcattttatagtgtctgggtccaatccaagtatctgttaggaacaccatacatagttttactctggaccgctagggaaccatttcaaaaatgaaagtaactggtttaaatttaacttagcaaaccatgcatttggatagttctaggtgaatagctttcaacaccagatttagatctcatttctctattaatttcattaatttttggagaataaaaatgattctggacatttcattaatcattacagagggagttttctctgtgtccccacaaggcatgattctgggtctatggtgacttaagagggccacacaacaatgagtatttaattttcctcagatgtatggctacaataaacatctataggtaaggtttacattcataaagagacctttttttttccaggaaaaaagacttttattccccctaaatcacaactcccctgtgtctgtccctcaaccctgatttctcttctaaaccgtaatttacaaacccatgtgcaaacccactgaaaggtgagaaggagcataagccagagacactgggaaccacagccaactacagggggtttttcatttttttgttttgttttgttttttggcaaaataaatcatgattatggctaggaaaatcagggatgtaagtaagcaaaaatttagaaactacatattgcatgtagtccccaaattcagaaacagtcatcgttaaacatcttttatttagtctttcagatatttttctacatatacctatttgcacatttcacaaaaaagagttattaactgtggacactctttgacttgcattttccacttatagctatcttttggtgcaaataagtaatatattttggagcttgctattcttctctttttatggtatttttgtgtgcacattcttatgctcttattcagttatttctgtagaatacaatttttgaaataaaaatactagattcaaaggtgtgaatatttttgaaggttttgtggtgtggtatcatgaaattaggtttcagaaagtttactccatagttgactttttctaccagctaataagagtgctcatttaccccacttaggccaactctaacaggcttctgttgctttgcatcctgacatatttatctttctggaaacagtgtcctaatttatttctgcagagctactgcctctcagttttggtccatgtggttctggtgaccctgattcttctgctgaaccagaaagtgcacacatcctcctggctgtggtgacccaatcagagccaacgtcttgcaatgaagcttttctttagacatctagaaataagactcttatgttttttttttccaatttgacttgaaacttaagacaatacacaggcctgagttgttggtactattttgctactacgtggagcttgagagtaatggtaacaccaagactggagcaagtggaaggagacatattgtccactgatgctatcagttgagcacaatgtgcagttactttttaagccagattcaccatgggctttttggctcctgaaccaatagatttcatttgttttcaagccactttgtactaggttttccatcatttgcaatcaaaaccagtatacaaacactggttatttaatttttcatttttgctaatctcagggataaaatggcttctagttgttttaacttggctatatttgtgattcttcccattttcatatacttactaatcacttctatttcttttgtaaattatcttttcatatttgttatctaattctttaaaatttgagtactttaattctcattattgtttgtggaattgttaaacaagactgaataatctgcccaaagtccatggacatgggggcccatgtaaaagctttgaaagccaccatcattatgagataaattatataatagtactttacataggctccaaaatacagcacagacacagctatcattgtcatggtcatcattatcatcatgatcaccaacttatgaggataagcaagaacacctactagaagtttctttccattcagcaacaaaattggtgtctttctagtcactcccttccctgactgtcacatagcagttcacagaggctcagttgcagaatgagaagctctgggccaggacctgccattgtatgcatccttgcatgggaactgggggctggaagaggagtgactgcttgataattatgagtcagtcaaaaccaccaactgtctgaaaaaaataggcctttttgtaactagtattgtcactaaaccaactcctccatgttttgtgcatacatgaaatctaggcaatacacttgtattcccaaaagcttccacttgaagagatctgtgctctttccaaatataaaccttacccgagaggtggtcatcttggccacacctcagagagggagagaggcagtcttgttgggttgggtggtcataatggggctcagggccaaatccccagggggtaggatagtgcagagaagatggcactctccagtgcttaataaaatgcacgtggtctaagctgcccactccctcaaaggcaataaaaaataggtactatttaaattgaagagtaactactgccccagggaatggacaggttgtcattggaatagccatggttaatggtcccagttgacaactgaaatgaatgtgctacctgaacaggaaagcttattaaccagatttcaaagacagtctttcccggtaaatccaaatttacaaattaaagccagtaaagacaccgaattctctaataatatgtggggtgcagtatattttagagctgggaataattccaaacagcaaatagttcaaaatttattttcaatttagcatatgcatgcatttgcttaaactgtcttaaaaatgagtaaaaaatactgtcagtttgtttcaatcatactgagatgaggaacaatgtttagcattgcatgctagaaaaggacacaggattgggagtcagggctgggtgaccgtcacaggactttcactaactgtgtggatcttgggcaagtctgtgtgccctgagactcagttatttaactttcttttaaaaaacatagtccagatgcagataacaaagcctgcctctaatttcccttacagaattgtgagaagctggtggagatgtttgttacaaaagtgttttgaaaatagagcaaatattattctttttaaggcatgatgttttcatagcatgtcaggcaacaggaaaaaactaagttaggattttattttattgtggggaatttatgtgcaaattattgtgcaatttaatgaaaataagccaatgttttatacagaagtacccagaaaattaaataacactatacattgttcaaatagttgccttaatatattttattttctcagcataattagagttgtattatacaggtctttgagtagtcagtcagtgggagaagttaagacaacagatatctttttattaaaattattatatgaattatcgcaaattaatttttatggttctgtcacaggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtaaaagttggtatttcattattggagaagctgtttttcaaaactgaatcagttttgtgcagaaaggtgtagtataactgagagttcttcctcacacggggttcaaggaccagcttcagcaaaatcccgtcaagtggttcttacaaatgcagattcctaggccacaacccagatctgctgaaccaaagtttcttgtgaccaggaatctgcattttaaacaatcactgtgtttctttaaagtagtagaagctagtcattttctattcaaagcctcaaaatgcttgaatatcattgggctaagggattgtctcaagaaagagtctaacaggtgcacatttcatctgaataaagaaacagatttaactgtgtgacccatgatcacattagcggatagcacagtccaaagaaaataacataagacaagcattttgctgagaatgtaattgagaaagatctagaacttgtgattttgggacagggcagttctaaatggggcctatagtgagccagtttgggcacctgtggcatgatgctatgtatggtgtgtgtgtgtatgtgtgcttgtgcttgtgtgaatatgtattattaactggaaatttgtaaaagtattggaaaaatagtacttacgattttgtgtgtgtgtgtgatggagtctcgctttgtagcccaggctggagtgcagtggcgtgatctcggctcactgcaacttccacctcccaggttcaagcgattctcctgcctcagcctcccaagtagctgggattacaggcacgcaccaccacgtccagctaatttttgtagttttagtagagacggggtttcaccacattggccaggctggccttgaactcctgacctcgtgatccacctgcctctacctcctaatgtgctgggattacaggcatgagccaccgcacccagcggtaattacaatttttattaggtcagagagatgcttattaatcacgagccacagtttcatcttaatgatttttccttttgattaatatccagagtaagcttttctttgttgttcccatttccatgttcataactctttactcatcttcactctatgtgagtttaccaactagaaattggatagtcattctctgatcccacatgttaaacttgtagagaaaactcagattgtatgtgaggatcatcatattaaaagtggaggaaggttctagaattcttataaataatgaaattaacatgaaggtggacatctaagacagagggaagtcttccattaagtgcagactacaaggagttaataagcaagatgaacacgatatacaaatccagctcttatcactaagttaactttttaagtaaatgaaagtatttgcaaaaataattaccaattgagaacatagttgcctgaaagtttaagaacacaggaaaaatcattaactctttaatatggttgatttcctgtacttaaaaaatgtgagtgtaaagaaaacgcagtgatggagttagatattatggggtgttataaaattagctctaagagtgttctttccagcaagtattggggaagctatattattttccttattcctggttttatttgttagtgtgtagaaaatgctagacatttcctcaatgtatgtttattattctacttcctaagtaaagctacttttaaaataggtttggttgcttcttgaaagatagtgttacaggaaccctgccaaaagtacatcgaacaggtgcagtttctggacattccttgaagcaatgtggtcatcaaataagtggtaagttgtgaatttcttagctacatttgagttaatattggatctcgcttagaacagcttttgctcaaagtttgtactgctacagctttttggaaggcatcactcataaagataggagatggggcagtattctggacacaaaagagggacccatattcatctggacacttctattgtctttataaatcaacacatacttaatgagcctctattatttatgaggttagcgctcaagtgtaagatttgcagaaaatgaatccaaataattgtgtctcgtttccagataagaatttttaagaaaacacaagggaacatctctctcaagttcacttgagggtaatttttacatcagtgattctcaaccagcagtgattttgtctcttccactggggacatttgacaatgtctggagacatttttggtggctacaactagggaggatgctattagcatttactaagtagaggccagaatgtttgaatgctgaccaacattctacaaggcacagggcagtcgtccacagcaaataattttctggcccaaaacgtcaacagtgctgacatcaagaaactctgtgatataccactaggcccaaattgaagaactgagttctgcaaatcttgctaagaataatacttcctaaaggaaacttgaggactaggatgctagagaactttgattctgacatctgaagctactgatgtcttgggaaacagtttccaatgctatcctaataaatttaagacaaatgaactatttctcaaacatgactgggactgataagaaagtgaaaagtgctgaaaagattcaactgatgggttgtcagaatcttaaaataactgctgttattctatgtatgactatatatcattactattttattttcattatgcacaattaattttgtaggttcaaatttcagatgtttttaaatttgtcatcctttcctccctcattgatatcacctcttcaatacgtacacactttgagcctgctgtttgcattttaaccagttatcaaaggatggcaatgccttcattataaatgtgggcctgacttagccagtataataggtgtagtctacgtgaggtggagtacatttcctattttaaaagatcaatttttatgttaatccaatttggtataaattattcgagtaagtgctatttctgattgttgtatcttgtagcaaaatttaaagaaaaagtaatttgtgcctttctcaatattcctgttattgttcatgtattctaaaactcactgttactcacttagcttgcttttaatgttttttaaagtgaaaaattgttcccaagtacataaaatctctacactcaagaacaattctagtcaaaagcatttagagcttccgtatgaacacttaaagagtttttatttgtaagagtcgcatcccaactcttagcctgttcttttctcacatgcagaaaaataggaaagagacttcgtttccacagtctgcaaattcctgtgtttaagaaccacagtgaataatccacctccctgcccaactcatcgtactgtcatatagttttcctgacagtttgtgtattttctgtctttcccacccttaaatttagttttatgacttcaaccatacttcttaggagtggaaaggtatctgtagtagattatggtttattccacataatcttggggaataaaactttaaaaaagtatacagtttatacttctggttacattacttccttaaccaaaagtctaaccaagaaatttgaatctttaaaaaaaaaagaggccgggcgcggtggctcacgcctgtaatcccagcactttgggaggccgagacgggcggatcacgaggtcaggagatcgagaccatcctggctgacacggtgaaaccccgtctctactaaaaatacaaaaattagccgggcgtggtggcgcgcgcctgtagtcccagctactcgggaggctgaggcaggagaatggcgtgaacccgggaggcggagcttgcagtgagtcgagatcgcgccactgcgctccagcctgggcgacagagcgagactccgtctcaaaaaaaaaaaaaaaaaaaagagcctaattttgcttcactgtctgtgaaaagaattatctgtatcttttgcatgtaagacaaatctcaatgaaaagggtgcttaaatagaagttaacactattttaaagcaagaatggaagtggtttcatcatgcgtaaacaacaactctccacattttgtaatgattgatctggatgcaatttgtcatcagacaggagaagtcgaaagcaaagaaataacactgggagatagagaagctctttcattcaatgcgaaaggtcaaaggcacatcagtttctttaataatgcaaacctcagcacacattatcagtgtcctcattattattgccttgtttatttcccactgctcattgataatttcaacgtgaaatttacctgtattgctgcatgcatcttgcagtttaagaagtgaagtaacccaatttcaaagctagtgctttagggaaaatattggattgtatttacttcaagcagagttcgataatttatgtacataataaaaattttaaatcccttagttaatatagcagttgccaaaactgggctattatcattctaaactaccaacccaaatggtagtgggtatctaatctacctctagaaagaaaatggactgtatttgctctatgtatttttcttgtacagcttgccatcgagacatttataaaggagttgatatgagaggagtcaattttaatgtgtctaaggttagcagtgttgaagaatgccaaaaaaggtgcaccagtaacattcgctgccagtttttttcatatgccacgcaaacatttcacaaggcagagtaccggtgagtacaattcaaggtgtgtgttctttgtattggtgcctccaggatttcactgtattcttcttaacctcttttgttcccaaactaaaaaccaaacagggcttttattctaaccactttcctcatttacttactctattttatttttttatcttttatttatttatttatttattgagatggagtcgcgctctgtcgcccaggctagagtgcagtggcgtaatctcgactcactacaacctccgcttcccgggttcaagcgattctcctgcctcagcctcccgagtagctgggattacaggcgtccgccaccatgcctggctaatttttgtatttttagtagagtcggggttttactatgttggtcaggctggtctcgaactcctgatcttgtgatccacccacctcagcctcccaaagcgctgggattacaggcatgagccactgcgcctcgcccttcattttttaattaaataattcatttaatttcatttgtttctctactcttttcccctggcatgtaattgtaccgcatcttcaaagcctgacatcctcttcccctacttctccaaagctgattcttgcaggtctttcctcaaataccgtcccctccaaaagcccatttctgagcattctcttttaagtcacactcagctctgtttatttcattcatagagctaatcacaatttgatattaacttgtgatttttttccttatttttaaatcttatttttatttgcatagatgtatggggtacaagtgtaattttgttactttgatgtattttacagtggtaaagtctgggcttttggtatatccatcactggagtcatgtacattgtacccactaagctatttttcaacgcccgcccccttcccacctctctgtcaccttcccagtctctactgtctatcgctccatgctctactcaattttggtgtcattttttcctgaagcaaaattttggtgtcattttttctgaagtcattttctgaagtctgtgtcatttttcgctttcctgaagcgaattttggtgtcatttttcctgaagttccgtttgccccacacataggccttgcttatagaatgagggtttagtgtcacggagtctcctgcctcattctcaccctaacttttcctttacccctttgcgaggggaaggatgtccattaggttaataatgcagacccctaacccactcattatcagggtcattgtttttccactgtgcattttaatactaactgttacctgcactgctccctgcccctcaaagtgcagaaagcaaagtaacctcttttcttcccattcaggaacaattgcctattaaagtacagtcccggaggaacacctaccgctataaaggtgctgagtaacgtggaatctggattctcactgaagccctgtgccctttcagaaattggtaattgtaggactacttcactttgtgattgtggtaggtggaataggagcccccagagacgtccctgtgctgagccctgggacctgtgcgtgtgttcccatagctggcaaaagcgtttctgtcaatggcatgcagttacgggcttcgagatggggagtttactctggattttctgaatgggcccaatgtactcacagggttgagtgctcacaaggctcataagaaaaagagagaggcggaaggctcagagccagagagagaggtttgaaggtattacactgctggctttgaagatgaaggtccgtgagccaaggaatgcaggcggcctctagaagttgaaaagggcgaggaaagagtttccctgtggagcgtcctggaggaagaagccctgctgatgtcttgattttagcccagtaagacccaatctctagaacagtaagataattaatttgtgttgtttttaaccactaagtttgtggttatgcccctagagcagcagttataggaaactagtacagtgatactgttagagttataggacagtgatataggacagtgatactgttatagttataggaaactagtacagtgatactgttagagttataggacagtgatataggacagtgatattgttatagttataggaaactagtacagtgatactgttagagttataggtacagtgatattgttatagttataggaaactagtacagtgatactgttacaggtacagtgatataggacagtgatactgttataggaaactagtacagtgatactgttagagttataggtacagtgacataggacagtgatactgttatagttataggaaactagtacagtgatactgttagagttataggtagagtgatataggacagtgatactgttatagttatagaaaactagtacagtgatactgttatagttataggacagtgatataggacagtgatattgttatagttataggaaactagtacagtgatactgttagagttataggtacagtgatataggacagtgatattgttatagttataggaaactagtacagtgatactgttagagttataggtacagtgatattgttatagttataggaaactagtacagtgatactgttataggtacagtgatataggacagtgatactgttataggaaactagtacagtgatactgttagagttataggtacagtgacataggacagtgatactgttatagttataggaaactagtacagtgatactgttagagttataggtacagtgatataggacagtgatactgttatagttataggaaactagtacagtgatactgttatagttataggacagtgatattgttatagttataggaaactagtacagtgatactgttagagttataggtacagtgatataggacagtgatattgttatagttataggaaactagtacagtgatactgttatagttataggacagtgatataggacagtgatattgttatagttataggaaactagtacggtgatactgttatagttataggtacagtgatattgttatagttataggaaactagtacagtgatactgttagagttataggtacagtgatataggacagtgatattgttatagttataggaaactagtacagtgatactgttatagttgtaggacagtgatattgttatagttataggaaactagtacagtgatactgttagagttataggtacagtgatataggacagtgatactgttatagttataggacagtgatattcttatagttaccgtggtatagttatagttaaaggtacagtgatattgttatagttataggacagtgatattgttatagttataggacagtgatgtagttacagtgatataggtacagtgatattgttatagttataggacagtgatatagttataggacagtgatattgttacagttataggtacagtgacgttgtaatagttataggacagtgatattgttatagttataggtacagtgatgttgtagcaaaactgtaaggtcattccttggttgtgtccctatctagtgaaatgactctaccaggggtagggaaataaaactctgtcgtttcacacataaaggtaatttcaatggaattatccagaaaattgccatgacattccacctcatttagcatgtcaggatgttaatgacaagatgttactaaaagcaaatcccttacggccagttttccgcagtactgggtgctggctctgtgcctggccctgtattgggtgctgggctaggatttccctgtggaagattgggaaggttggttacaaggtggctattttcctgtctcctctttgcgacagcacaccttctccatggtgtgtgccaggttcacgtgtactggtgatttaattttaacgttcatattatttttttctgggagagtttttgaaggctgccaggaggcaggactcgatgcaaacatgctccattctgtacccagccctgttgctggaaggatttgctgcacttacccagggaacaggcaagctcgtgctgtggctctgggctgtcacagctgctgtccacacctgggagagcaccctggatggctcatctgtgtacttgctttcttgttaaattgcagtgagttcacatgtgatttaatcggatcaaatggcctttacagactgataaaaatatggctgtttcaggtggtgttttgagctgctaagggcgtggcttttcactgagtacgtggtccccgttcctcagggaacacccagtagccacatgcctcctaaacctagagtagggctgtctcctggcctaactgcccaaatgagattcataagttagggatgatctgtagttatcactacagatttgtccttggtttcaccaaggattttcctaattttacaaacaaaacccctaaggctcctggaaggagggtagaagtgaaggtgctccgggggcaacacagctgatgagctgaaccagaactcgacccttgggtcacacacatttcacagtgctcactccaccttttgtttttttaatggatttaatggtgttttaaagcctcctgcctctcaacacatatgaattcattatatttacagatttccttctcttgtggtccatcttcctgcatagatcttgagagatgtcaggctaaccacgtttcctcagttaatttaacaaaaccatttgcaactctgacatggaaaattcctaccatgtgacttattaatttatcaattgagatggtacacatattttcaagccaaaaggaggaaaacataaattggaaaaaaaaggtttttttatttttatcacctctggggaagaaagtctgataaacgaagctggttgataaaattgcaattaggggaagcaacatcatggtttctgttcggaggctaaccagatggcatacttgaaatagagaatgtcctagaaatcaactggttgcttggccaaaatatctataaatagtgcccaacatattagataggaaaagcaaagtaaaaacaattttaacaggttaggacattgggctgaagtattgcatatatttaatgtcatgtgcgtccgtgtgaagagaccactaaacaggctttgtgtgagcaagaaagctttttaatcacctgggtgcaggcaggctgagtccgaaaagagagtcagtgaagggagatggggtggggacgttttataggatttgggtaggtagtggaaaattacagtcaaagggggttgttctctggcgggcagcggtggggggtcacaaggtgctcagtgggggagcttctgagccaggagaaggaatttcacaaggtaacgtcatcagttaaggcaggaaccggccattttcacttcttttgtctttcttcagttacttcaggtcatctagatgtatacgtgcaggcctgggcccagaggcctgacattcctgtcttcttatattaataagaaaaagaaaacgaaatagtggtaaagtgttggggtggcgaaagtttttgggggtggtatggagagataatgggcgatgtttctcagggctgcttcgagcgggattaggggcggcgtgggaacctacagtgggagagatgaagctgaaggaatattttatggtaaggggtgatattgtggggttgttagaagcagcatttgtcatatagaatgattggtgatggcctggatatggttttgtgtgaaatgagaaactaaatggaagacacaaggtctgaataagagaaggagaaaaacaggtgttaaaggactaagaattgggaggacccaggacatctaattagagagtgcctaagggggttcagtgtaattacttgcttggttggtgagtttttgggctctatccttgacagagtcctcctttttaagttggaggctgagcttggtgaggtgtgtttttaaaagaccattagtctgttctacctttcctgaagattgaggatggtgaggggtatgaaggttttactgaataccaagagcctgagaaactgcttgggtgatttgactaataaaggccggtctgttatcggattgtatagagatggaaaggccaaactgaggaattatgtctgacagaagggaagaaatgaccacggtggccttctcagaccctgtgggaaaggcctctacccatccagtgaaagtgtctacccagaccaagaggtattttagtttcctgactccgggtatgtgagtaaagtcaatctgccagtcctgggcgggggcaaatccccgagcttgatgtgtagggaagggagggggcctgagcaatccctgaggaggagtggagtagcagatggaacactgagcagttattttttgaggatagatttttacgacggaaaggaaaagtgaggttttaagaggtgggttagtggcttgtaacttacatggaagagtttatgaaatgatgacagaatagaatgggcctgtgaggctggaggagatattttccttggtccaagaattatttgccttgtgtgggaagagattgataggtggaagtttcaatgggggagtagatgggagtgacagatgaggaagaaaaaaactggctgtgagggatagaagttggaatgctcgctgcttttttagctaccttatcagcataggcattgtcctgagcagtgggatctgatgccttttggtggcccttgcagtgaatggcttcagcttcctttggaagtaaagtggccttgagaggagtttttattaaagaggcattaagatggagaacccttgtgtagtgaggaaacctccttcagcccatataaccgcatggtggtgcagaatatggaaggcatatttagagtcagtataaatattgacacgtagtccctttgcaagagtgagggcctgagttaaggcaatgagttcagcttgctgagaggtagtggagcggggcagagcagtagcctcagtgatagatgtggaagatactacagcatagcctgcctttgctggtgagtggtgattaggcctggtggaactgccatcaataaaccaagtgtgatcagggtaaggaacaggaaagaaggaaatatggggaaatggagtggatgtcaggtggatcagagagatacagtcatgggggtgggggccagcctaaaacagtaaggtcaagttgtttgaacagaaaggctacagggcgtggtcctggctcttgtgtaagaattttgactgcgcagccctgcacttcggctgtgtgtaatgaaaagggttgggatgagttagggagagctagtgtgggagcagtttctagggctgtttttaaggaatggcaagaggagtggctaaaggatttaggatctttggggtcagctagctttgcttttgtgagtttatataatggtttagtcaggatggtaaaacttagtatccaaaggcggaagtacttaaccatacctaggaagaaaaggagttgttttgtagaaggggttggggtttgggagatgagccagacacaatcagcagggagagcacatgtgttttcatgaagaattatgccgagataggtaatggatgaggaagaaatttgggcttgactgaagtaatgggggctgtcctcgaagccttgtggcagtacagcccaagtaagttgctgaggctgacgggtgtcagggtcagtccaagtgaaagcgaagagaggctgggatgaagggtgcaaaggaatagtaaagaaagcatgtttgagatccagaacagaataatgggttgtggagggaggtattgaggataggagagtatatggctttggtaccatggggtgaataggcaagacaatttggttaatgaggcacagatcctgaactaacctgtaaggcttgtccggtttttggacaggtaaaatgggggaattgtaaggagagtttataggcttcaaaaggccacgctgtaacaggtgagtgataacaggctttaatccttttaaagcatgctgtgggatgggatattggcattgagcggggtaagtgtgattaggttttaatgggatggtaaggggtgcacgataggttgccaaggagggagcagaggtgtcctatacttgtggattaaggtggggacacacaaggggaggatgtgaaggaggctttgaactggggaaagggtggcattgaggtgtggctgtggcctaagaacagtcagggaagcggataattgagttaaaatgcctcgacctagtaagggagctgggcaggtggtgataactaaaaaggagtgcataaaagaatgttgtccaagttggcaccagagttggggagttttaagaggtttagaagcctggcggtcaatacctacaacagttatggaggcaagggaaacaggcccttgaaaagaacgtaatgtggagtgggtagcctctgtattaattaagaaggggatggatttaccctccactgtaagagttacctaaagcatctgtgatggtccaggaggcttctaaggtgatcgggcagcgtcagtcttcagccgctaagccaagaagatctgggaagcagtcagtcagagagccttgggccagagttccaggggctctgggagtggcagccaggccagttagacagtccgatttctagtggggtcccacacagatgagacacagcttaggaggaatcccaggctgcgggcattccttggcccattggccagatttctggcacttgaaacaagatcctgatggaggaggtcctgtaggaatgcttgaccactgcagtttaggcattttgaagtttttgtgtgtgctggagatgtggctgggttttgtctcacagcagaggcaaggaatcgcaactcagaaatacattgctacttggctgcctctattattgtacatcttgaaggcgaggttaattaagtcctcttgtggggtttgagggctggaatctaatttttggagtttttttttgtttgttttttggttttttttttttaatgtcaggagctgactgggtgataaaatgcatattgagaataagaggccttctgacccttctgggtctagggctgtaaagcgtctcagggttgctgccaaacgggccatgaactgggctgggtttttcatatttgatgaaaaagagcctaaacgctaactgatttgggagaggtcggataaataaaaaggaacattaatcttgactatgcctttagctccaaccacctctttaagaggaaattgttgggcaggtgggggagggctagtcgtggaatgaaactgtaagctggaccgggtgtgaggaggggaggtgatagaaggattatagggtggaggagcagaggctgaggaagaattgggatctggcttggcctggcaaggagcagcctggggaggaggggagaggtcagatgggtccatagaaaaggaggattggaaagactcagcaacacttggggttgggattgagaggacagatgggttgggattgaggggacagatgggagggaaagaaggaagatttgggacaagttgcattgggaacagagactagggagggaccaatgtgtaaaagaatgcctggacgtcaggcacctcagaccgtttgcccattttatgacaagaattatctagatcttgtaggatggaaaaatcgaaagtgccgttttctggctatttggaaccattgtcgagtttgtattggggttaagcagcattgcagaagaaaataaggcatttaggttttaggtcaggtgtgagttgaagaggttttaagttcttgagaacataggctaagggagaagaaggaggaatggagggtggaaagttgcctatagtgaaggaggcaagcccagagaaaagagagggtagagacatggagagaaggggtgggggggtgcttgcccccaggaaagtggttcttgccactaagggtgaaggatcaaggcaggcattcgcgcggtgatcagatacctctgaaacgtgggtgaataatcaagcaggtgtccctgcagtgattaaacagcaaggaaagactatcttcccaagtccatgaccagtgccagagttttgggttcatggataaaacgcgtctcctctgtctctaccagaaaatgaaaggaattgaaattaagagaagggagagattgaaggatggcgccaagattgaaaggaaaaagaggttgagggatagggagagaggttggataagagagtaaaaagaggctgcttacccaatttaaaatcggtgagatgttccttgggcttgttggtctgaggaccagaggtcatgggtggatctttctcatggagcaaagagcagggggacaggggattgatttcccaagggaggtcccctgatctgagtcacagcaccaaatatcacgtgtgtccatgcgaagagaccaccaaacaggctttgtgtgagcaagaaagctttttaatcacctgggtgcaggcgggctgagtccaaaaagagagtcagtgaagggagataggggtggggacgttttataggatttgggtaggtagtggaaaattacagtcaaagggggttgttctctggcgggcaggggtggggggtcacaaggtgctcagtgggggagcttctgagccaggagaaggaatttcacaaggtaacgtcatcagttaaggcaggaaccggccattttcacttcttttgtcattcttcagttacttcaggccatctggatgtatgcatgtaggcttgggcccagaggcctgacatttaacatggataaatgtaaagttcttagaatcatacatacactttggaaaagatggggcttaatcgcactttataagacttgaaggatgtttgagaatcactatgaaactgctgaaaataccaagaaaatttaattcttatgtatataaataatgtgtctgttttacatgaatcccttctacaagcttggtatttaatatggcatatattgttttttcatagtagatttaaaatttttgatatctaatttagataacataaaattaaccctttgaaagtgtacaactccgtggtttttagtatatccacctgattgcacaacgatcaccactgtctagttccagaacatttttatcaccacaaaagaaaggctgtatccaggccgggcacggtggctcacgcctgtaatcccagcactttgggaggccgaggcgggcggatcacggggtcaggagattgaaaccatcctgggtaacacggtgaagccctatctgtactaaagatacaaaaaaattagctgggcatgatggcagatgcctgtagtcccagctactcaggaggctgaggcaggagaatggcctgaacccaggaagcggagcttgcagtgagccaagattgcgccactgcactccagcctgggcgacagagcaagactccatctcacaaaaaaataataaaaataaaataaaaaaaaaaaagaaaggctgtctttctcctttcccattggccgtctttctccattccctactcctccaatcccctggcaaccactaaatctactttccatgtctgtggacttgactcttcgggacattttacataaatggaatcatgcaatgcagcacattttgcatctggcttttttcacctggcgtgttttcaaggctcattcgtattctagcatatatcaatactttgttcctatttaggactaaataagattctattgtatgaataaaacatattttgtttatatacttagtttgatgaacatttgagttgtttctggatttttttttttttttttttgcctcttatgaataatgctgctatggacaacagtttttgggtaggcatgcattttaaattctcttatgtatatacttaggattaaaattgctggatcacagagtaactccatgtttaactttttgatgaattgccaaactgtttttgagagcagctacacaatgttacattcttaccagcaacaattgagggcttcagtctctctacaaccttaacaacacttgttattgtctttgtaattattgcctttctaggcagtgtgaagtggtgtctcactgtggttttgatatgcatttccctaatgactaacaatgttgtgtatcttttcgtgtgctcattttcaatttgaatacattctttgggaaaatctctgtttaaatcttttggccattaaaaataattgggttattttcattgttgagttgtatgaactctttatatactctggatactacactcttatgacatatattattttcaaaaattttctgtgcatctgcaggtcatcttttcactttactgatggtgttctttgaagcaccaaagtttttaaacttgatgaaatccagtctggctttattcttgcacgtgctttacctaaaacgccaaaacctaattcatggttttgaagatttttgcttatgatttgttcttagaggtttatagttttagctcttacatttaggcatttgatgcattttaaattaaattttgtatatggtgtaaagtagcaatccaacttaattcttgcatgtgggtattcagttattccattgtcttgaaacccttttcaaaatcaattgtctataaatgtaagagtttatttttggaccatcagttctatcctgttgacctatatatgcctatcctaatgccagttacacacagtcttgattaccataactttgcagtaagttttgaactcagacagtctgagtgcttttattttgtcctttttcaagattaatttggttattccggatcttttgcatttccatatgaattttaggatggttgtcaatttctgcaatagaaaagcagcaggattttgatagagagtgcattgaatctgtagaccaatgagtatcaatggaattatgtgttatcaaatttagtaaactacataaacgatatgtacacaactaaaacaaaactaagatataagatcatattaatgtaatgataataaaagtggattgtctcctgttctaattttaataggcacaaggcatttttgttaatcacttcttattaaagaattttttataatattcaggaaaatacaacaaaaaaacccttacattcctaaggcctcagagtcagagtaatataaaggaaatgtaaacacatgctgagtaatgcaagcatttggcaatggtggtgactggagctgggagcacagcttttatttctctgaaataggaatttgccccttagagttagaccaattttgccttcctctaaatggcaaacagtttggagatactttaaaggacattttttcacttaggatgtttagtactatgaataataaatagtcacaatttccttaactatggtgacaaaatacaagcaaatttagcctcatgtcatttcctaaggaacatcttctctctgtgagttcacaggttgccacatgaacatcttccagcatcttgcgttctcagatgtggatgttgccagggttctcactccagatgcttttgtgtgtcggaccatctgcacctatcaccccaactgcctcttctttacattctatacaaatgtatggaaaatcgagtcacaaaggcgagtatgcatggaaaatcgcatcacaaaggcgagtatgcatggggagcacttgctgctgtactttcatcacttttatagtctgagttcttaaaagtttcgttcatttccctcaaaacacttgaacctgcagtttcagtaggtactgttctgccaggtgcagattagttaagagattagcagacttctctgcctatcttctcttactttaaaacaaatgttaccattgaatcaaggaagcaatagccatgagaaaaaagaaggatctgacgcctttgaatgaagattcaaaacatgatcttcatgttttgtattagcttggagtaaaatccacttgctggcaatatagcccttaagcttgttgcctcttctctttgtttcagaaactagagccctgtttattctgatcaaggctctggcccactgtctttatctcagataacccaccctcttctgcacacagcatggagctaagagaagggtgtctagttatgtaatatcatcggcagcataaattcccagaatttgttctttgatttttttgtttgtttttccagttagaaggtggaacttcatcattgtcctcttttcaggttgtctgtgcctattagttttctccagagggagaggtggcttgatttacatttaatctctgcaatttattagagtcctgtagttggatttactttgaagagagtttcccagaagaataaaatttgctgcgttgctttttgggtgtgagctgcttttgtatttgcctaatgcctttaatgcaaatttctttctttctctcttgcttttttttaaaaaaaatagaaatgtttgtcttcttaaaacatctgaaagtggcacaccaagttcctctactcctcaagaaaacaccatatctggatatagccttttaacctgcaaaagaactttacctggtaatgtgatttgataataatattacataaaatgtaacctatttcatgacttttaacagcaacagtgatgaaacaatcctcaaggtaacagaaacttgtgtaaatgtcgttcattgcttttcccatctgatatctttttgtgtttataattgacacagaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtaaaggaaattttatttttcaaagacagttgacatgaccatttcatattctctttccccctgtgaaggcttactctttctactgttcatttcatctaggtgtaagtgtttcttaagattatctatggatggttctccaactaggattgcgtatgggacacaagggagctctggttactctttgagattgtgtaacactggggacaactctggtgagtaacctcactttttcgtggacctgtcagggatgtctgtcatgttgatagtttgcttagtcttaaggaattatgtgtcttgttctccttggttagaagggactttgattcacttctaattccaaccattagcgtcaacgctctcttttcagtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggtaagtgttggatgcatctcatccagagtcttatcttggcttttcattttgaaggatctatgatcagctgcttcaccgccatgtgactttatgaatagagacgtgttaaagcggggatggtattcacaacatttaacttatagggtccaagcactgaccaacctgaccattagaacagagtgtggtctctgtacagggcagatggcgctgagtgggtattctccacagaaagagaaacgaagacagtaccccactcctccaacccaccacccaccaccaatcccaccaccaattccaccaccaatcctgccacccaccaccaatctcaccaccaatcccaccaccaatcctaccacccaccatcaatctcagcaccaatcccaccaccaatcccaccaccaatcccaccacctaccccaccaccaatcccgccacccacgaccaatcccaccaccgatcccgccaccaatcccaccaccaatcccaccacctaccccaccaccaatcccgccacccacgaccaatcccaccaccgatcccgccaccaaccaccaatcccaccaccaatcccaccaccaatccctgatgtgttcttcaaagacttatttgtcaggcccatagaaatgttacttcttgctctttgattcataaatatactaagtcataataatttttaaaagtgagagtttcgtactctgtatatttcaatgtatataatttgatctatttcaatttattggtcaaatagtagacatgttaggtaagtcttaaaatactgaggctttggagttagacagaacatggcttaagtgacagctttgctgcttattagaggtgtggccctagaagatttgtaaatccctctgagctttatttgatctaaaatatgaatagtaatagtcccgaatttgtaacgttgttgggaagattaagtgacacatttaaaatgcttagtactgtgtgtagaacataaacacttcaaaaaatgtaaactgtgatttctatattcaataagaaatgtagaaatggacaaagcatataaaaagcaaaagaaatactagaagacacttgatttttctcaaaaataaacacaccaagtatttttgttttagtgaaattcatgcttacatgctgtatactaggattgaacatactgccaccaaaatatagcagtcggtggtacatgtgggtggagcaagacccctccaccttgtcatcgtgtgaaggggctctgccatacatgaccttgcatgtgactttaaggtggttggcctggaagaaaagtcccaagatgggaaatagtaggtgtcttttttactaaatgcactccaatttgggaccaaaaattttcattcttgaaggctcagtattgtgagtttataagagataatagacataaaagtgtaatgatttcattgcaaataaaaaaaggcccctttgcacctgatatctccatcatttttctagaattttgtgcacacatgccttgcactacttggtgatgataaagatttccagatctttgcacagaataaggctttgctttagatcagaattttggatgtacttagtatacattcatcttttaaataatctatttacattttcatactttccaaaatacagatatattttattttatttatatatttatttaatttattttttgagatggagtctctctctgttgcccagagtagagtgcagtggcacaatcttggttcactgcagcctctgcctcccgggttcaagcgattctcctgcctcagcctcctgagtagctgggattacaggcgcgcgccacacctggctaatttttgtatttttagtagagacgaggtttcatcatgttggtcaggctggtctcgaactcctggcctcaagggatccacccacctcggcctcccgaagtgctgggattacaggtgtgggccactgtgcccagctgtatagagatattttaaacaacactaaagtcctcctactttgactaattagaagagcattagaagatcagcctgacttcttgacagttctgaatttagtggagcaatgaggttcagctttggtgaatgagcttaatttttccatgataaactgctagtttcttcccactacagtgtctctcaaaaatgggacagcaacattctttttgttttcacttgcagtaagcatgatgcaattacataaatgtacacttttcaatttgttaaatagaatcttcagagattcactactgccgctattggtgatgaaaaattaccagaaggaggaattaggtaggagaaaatgtgtcctatgtatttccttcccagttctttgaaagagagtgataggaaaaaggaacactattgaaggaaggactgcccagtttcaaacaggtatttatttttctctcctaggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactggtatgtagcatatgtaagaaggtggagagcagaattgcgctggttgatattttcatatcagtttgaacaagagggcagacctagagagactgtcgtcgttttctgactggtggagttgagggaaacgtgagggttgctgggaagtgaagaccccgcgacttgccgtgaaatctcttctacttaaagagcaagacatgtgaattaattctttcagggagggatacaactgcatgcaggtgatggaaataatgggcgtgggaaatgtctgtgccgtctgagaggcactgggcttgctttgacaagagtagcagaactgtcattgctttgggcttagggatattcgaatgtgtgagggcaagtgggatcagatatctacttccaggtataatttgggtaggaaagagactcatgcagaaagaagccctggaaggccagagcatcgtggtcagaggtgttgcctttggagggtcattgctgccaggagccgaatacccactgtatccaataacattcatggtcaggaatggtggctcacacctgtaatcccaacactttgggatgcccaggtgggaggattgcttgaggccaggagtttgagaccagcctgggcaacacagtgagaccccgtctctacataaaattagaaaaaaacaattaactgggtgtggtggtgtgcacctgtagtcccagctagtcaagaggctgagacaagaggatctcttgagcccaggagctcaaggctgtagtgagccaagatcgtgccactgcacacaaacaattatgtgacctcgggcaagttgctttacctctttacacctcttaatttccttatctgtaaaatgaggatgataatttcttcctgggtctgttgtaataattaatacatcaaagcacttcatgtctggaacagtgaagataccctgctatgactattaaggatagtatacatggaataagacacaggaacttctaaatgcttttgaccatagatttaggttctgagttttaagaatttaactcaggaaattgtaacaccaaaaatgtcatgtgaaaaatggtggtgacaaattttcttgaatcattagccttagaggttgggcagaaagcaaaaaattattcttgatgctactctatagaaagagaagacagaaaaagagaaagatgtatttttaaagtctatatccataactttatttgaccaaactctaatttaaaaattatgtttcagaattccaaaaaccaatatgcctaccttccaaaggtgacacaagcacaatttataccaactgttgggtaaccggatggggcttctcgaaggagaaaggtaagcatgacgctttaaatattgcttctagagtaagtctcacatgttgaaatacatggagtgggtcgttttaatcggtttctgtctgaaattatatctaaactctttatctttcctatctatttattcccaaatatttattcagttattcttaaaaaatgtatttttgctttggcttgaaaaaaaattttagggagacttttaagcatcttacttcattataaagatcagttgcttgactttgcatgaagcagattgggcccttctagggctgacaagcccgtgcaagaccacccgctcctcagtgttagtagcgttcccgtctcccaaaaccatgttctcccttgatgctaatggccgggagcacaggcaggtgtgtcgtctcactatggagaataatatttgtgtcattctttacagaagaaggtagcttgccaaactgtctccatctttcccgattcagtcttttgttcaagtaattcacatttttagattttttattggtaatctgagacaagaagaaatttaaagtaatcttcactaagccatgaaagctcccaacattgttctccatgagagatgctggcctgcatttattcaaaaacaaaagacccctctgttgccaaagctcggagggcttttcagaaacgatatagttgtaaattataattttgaatatataaagcaaaaaaatgaaaagtgagaacttccaggctttggattgttgtaggtgataaatataaaatgggatttctggggggctgctactgagatgaggggatggcagaaaacatggaagcaaggtctctggtcagcccagggtgctgggcttgtcccaacaccacgtaggcaataagaggacagtacagggtgccgtctctctccctcttcctctctctgtctctctctctctgtgtgtgtgtgtgtgtgtgtgtgtaacactaccttcccaatttttactgtctatttgtattcaaagataaggtccttatgaaaaatacactgctctgattcactttaaaacttatttccatatttattatttattgtgggaataataatattcccaatattattatttattatttaagttattattattaacttccctctgaggttatatattggttactcacaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaaggtaactcatgagattatgaaaaacacaataggctgcttgagaaaattcatttcaaaatatattttccaatagcataattcaatcatagtttttaaaaaaattcagagacaaatgatctgataaattgataagcaacttttaacaaattgaatatacataatatatatttatattatttatgatatatgtcacaatctatgcatgtgctatttaagaggggcaaatatacatgcaataattgtgctagaatataaaaacattagacttcatcattgggatgatgatatcaagatttctttgttagatttatttcagatagaaaaggggatacgaaaaatgcaggcacatgagatacttggagaactttaagaaagagtgagtgtgtgtgtgtgtgtgtgtgtgtgtgtgtgtgtgtctggcaagcaaggtcttgcacacacacagcactttgggaggccaatgcaggtggatcacttgagcctaggaatttgagaccagtctgggcaatgtgatgaaacccatctctacaaaaaaatatgaaagtatctgtgtgtgtgtgttgctgtgtagtggactacagaactttagaggcagtcacttatttgaatcccattgtcgtaactttctactattttatttttccactgtgactcagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatggagagtggcatcttctttgcatcctaaggacgaaaaacacagtgcactcagagctgctgaggacaatgtctggctgaagcccgctttcagcacgccgtaaccaggggctgacaatgcgaggtcgcaactgagatctccatgactgtgtgttgtgaaataaaatggtgaaagatcacgattagcaagtgttttcttctggttgtgaaacagaactgaaagtaagtggttgaggttccagcacagttcctgggatccctctaattgcactgcttcctctggaactcagtatatctcaaagatgtaatttcctctccgtgctgcacctggtcggccactgaaacccactattgcctgcttcacgtgtggcaaagagctagcgggcttgggttttgttctgccgagaggaagggagaacacccacttttataagaaagagatgggttacctgaacccatgggcacctttgcctcttggcctcctaactttgctaccagggcatggctaggagggtccaggctgcgcgtgctgaggagctcgaggggctgcagcattgcacagccttcatggcaggcaaggaatctgctttgcaaggggcattagccctggaggctcagtggatatgggctattgcaatagtaattcaaggagcatttttaggcctggcgtggtggctcacgcctgtaattccaacagtttaggagatcaaggcaggtggatcacttgagcataggagttcgagactagcctggccaatgtgacgaaaccccatctccacaaaaattagctgggcatggtggtgcgcacctgtaatcccagctcccccagaagctgaggcaggaggaccgcttgagcccggggatgtcgtggctgcagtgagctgagatggcaccactgcatcactgcattccagcctgggcaacagagtgagactgtctcaaaaaaaggaagcattgttaggtataaattattattattattattattagtagtagtagtagtagtagtagtactgagacggagtcttgctctgttgcccaggctggagtgcagtggtgcaatcttggctcactgcaatctctgcctcccgggttcacgccattctcctgcctcagcctccggagtagctgggactacaggcacccgccactgtgcttggctaattttttgtatttttagtagagacgggtttcaccatgttagccagaatggtctcgatctgctgaccttgtgatccacccgcctcggcctcccaaagtgctgggattacaggcttgagccaccacacccagccctgttaggtataaattatttcataaaattcagacttgtaaatttatggtagcctttggaatgggtgatagatgtcctatgccacacaaattcctcacatgccgaggctcaccgtaactgataccagctcgcttgattcagttcagttaaactgaacaacatttacacagaattggcgatttacaaaatcttatgtaagttaagtagaaaaacagaaaaaagttttctgaaagagtttgggtttttccttcaatgctcaagacagaggtccccaacctttttggcaccagggaccagttttgtggaagacagtttttccatggaccagcatggtggtgggggatgattctggaatgattcaagtgcatgacatttattatgcactttatttctattattactacattgtaatatataatgaaataattatgcaactcaccataatgtagaatcagtgggagctctgagcttgttttcctgcaactagacagtcccatctgagggtgatgggagacagtgacagatcatcaggcattagattctcataaggagcctagatccctcacatgtgcagttcataacagggtttgagctcctatgagagtctcatgctgctgcggatctggcaggaggcagagctcaggcggttatgcttgctggcctgccactcacctcctgctgtgcggcctggctgctaacaggccatggaccaggtactgttctgggccccgggggttgggaacccctgctcagagacacaccgggtggtaggaggagctaaaggtggagagggtgaaggaaaatatgaggtctgggctatccacaaaccagatagcaggaaactgaagagttaaacatttacttcagaattgaatggcttttgtaaaatctggctagattccatgaaacgattttaaaaatgcactatttaaattttgcctttgcatgcgatgaagacattagtctttgttcatgtggatgtttttttatgtttaaaagggaaaaaatggtttgcaatgaaacttttatctcagtctttgagtattgatcatggggtgttggaacaggactttggaatgcttgcagggtaaacctttggcctctgttagtcagggaatgacctagtttggcaaaacagaggagagttttgaaatatggaactttcccgaggcatacattgtcattttaaagtggtcaatcaaagcccagtaggactgggctggtgtcttggtgactcactgtgtgctcatatacaggggtaactgaggagcccttcacacaggtctagcctcgtgggactaaaaagtgtgacatgggctaggaaaatgcgaggctgggatgccttcactcccatgaggaagcgtgacgggaggaggcgtgggccactggcagttctacttcacaaaggctgctggcagtgtcaatcctgcaagctggccttgccctcctgtggcggcagtgtacacgtggcatgcaagcacatgcacaagccacctggctccaaggtcagccaagggctccaacatctgtctcagtccc

SEQ ID NO: 2 - RNA, 692 nt, Homo sapiens
  source 1..692: mol_type = mRNA
aattccaaaaaccaatatgcctaccttccaaaggtgacacaagcacaatttataccaactgttgggtaaccggatggggcttctcgaaggagaaaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatggagagtggcatcttctttgcatcctaaggacgaaaaacacagtgcactcagagctgctgaggacaatgtctggctgaagcccgctttcagcacgccgtaaccaggggctgacaatgcgaggtcgcaactgagatctccatgactgtgtgttgtgaaataaaatggtgaaagatcaaaaaa

SEQ ID NO: 3 - protein, 638 aa, Homo sapiens
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTSNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 4 - DNA, 20 bp, synthetic construct
  source 1..20: mol_type = other DNA
tgcaagtctcttggcaaaca

SEQ ID NO: 5 - DNA, 20 bp, synthetic construct
  source 1..20: mol_type = other DNA
  modified_base 1: mod_base = OTHER; note = 2'-O-methoxyethyl thymidine 3'-phosphorothioate conjugated at the 5'-position to triantennary trishexylamino-N-acetylgalactosamine-6-aminohexyl via a phosphodiester bond
  modified_base 2: mod_base = OTHER; note = 2'-O-methoxyethyl guanosine 3'-phosphorothioate
  modified_base 3: mod_base = OTHER; note = 2'-O-methoxyethyl 5-methylcytidine 3'-phosphodiester
  modified_base 4: mod_base = OTHER; note = 2'-O-methoxyethyl adenosine 3'-phosphodiester
  modified_base 5: mod_base = OTHER; note = 2'-O-methoxyethyl adenosine 3'-phosphorothioate
  modified_base 6: mod_base = OTHER; note = 2'-deoxyguanosine 3'-phosphorothioate
  modified_base 7: mod_base = OTHER; note = 2'-deoxythymidine 3'-phosphorothioate
  modified_base 8: mod_base = OTHER; note = 5-methyl-2'-deoxycytidine 3'-phosphorothioate
  modified_base 9: mod_base = OTHER; note = 2'-deoxythymidine 3'-phosphorothioate
  modified_base 10: mod_base = OTHER; note = 5-methyl-2'-deoxycytidine 3'-phosphorothioate
  modified_base 11..12: mod_base = OTHER; note = 2'-deoxythymidine 3'-phosphorothioate
  modified_base 13..14: mod_base = OTHER; note = 2'-deoxyguanosine 3'-phosphorothioate
  modified_base 15: mod_base = OTHER; note = 5-methyl-2'-deoxycytidine 3'-phosphorothioate
  modified_base 16..17: mod_base = OTHER; note = 2'-O-methoxyethyl adenosine 3'-phosphodiester
  modified_base 18: mod_base = OTHER; note = 2'-O-methoxyethyl adenosine 3'-phosphorothioate
  modified_base 19: mod_base = OTHER; note = 2'-O-methoxyethyl 5-methylcytidine 3'-phosphorothioate
  modified_base 20: mod_base = OTHER; note = 2'-O-methoxyethyl adenosine
tgcaagtctcttggcaaaca